(19) United States
(12) Patent Application Publication
(10) Pub. No.: US 2025/0248758 A1
(43) Pub. Date: Aug. 7, 2025

(54) MEDICAL DEVICE FOR MAPPING AND/OR ABLATION

(71) Applicant: St. Jude Medical, Cardiology Division, Inc., St. Paul, MN (US)

(72) Inventors: Andrew Oliverius, Zimmerman, MN (US); Jodee Maes, Golden Valley, MN (US); Brian Monahan, Elk River, MN (US); Jacob John Daly, Ham Lake, MN (US); Salo Arias, Brooklyn Park, MN (US); Evan Fick, Maple Grove, MN (US)

(21) Appl. No.: 19/042,373

(22) Filed: Jan. 31, 2025

Related U.S. Application Data
(60) Provisional application No. 63/548,667, filed on Feb. 1, 2024.
(60) Provisional application No. 63/649,082, filed on May 17, 2024.

(51) Int. Cl. A61B 18/14; A61B 18/00; A61M 25/00

(52) U.S. Cl. CPC A61B 18/1492; A61M 25/0053; A61B 2018/00172; A61B 2018/0022; A61B 2018/00267; A61B 2018/00351; A61B 2018/00577; A61B 2018/00613; A61B 2018/00773; A61B 2018/00839; A61B 2018/00875

(57) ABSTRACT

A medical device includes a first shaft and an expandable end assembly configured for mapping and/or ablation. The expandable end assembly includes a basket with a plurality of splines. The basket splines are coupled to a proximal coupler and a distal coupler. The expandable end assembly may further include an expandable balloon positioned within the basket and coupled to the proximal and distal couplers. A shaft extending between the proximal and distal couplers may include a pattern cut into the wall in the section of the first shaft positioned within the expandable end assembly. The expandable end assembly may further include a sensor configured to measure a characteristic indicative of an expansion state of the expandable balloon.

### CROSS-REFERENCE TO RELATED APPLICATIONS

This application claims the benefit of priority to U.S. provisional application 63/548,667 titled “Medical Device for Mapping and/or Ablation” filed Feb. 1, 2024, and to U.S. provisional application 63/649,082 titled “Medical Device for Mapping and/or Ablation” filed May 17, 2024, the contents of which are incorporated by reference herein.

### BACKGROUND

Medical devices, such as catheters, may be used to diagnose and treat cardiac arrhythmias. Catheters may include an end assembly for mapping and/or treating a target site of a body lumen. To reach a target site within a patient, a catheter may be advanced through a body lumen that includes one or more bends.

### SUMMARY

The present disclosure relates to a distal coupler configured to be secured to a shaft of a catheter, the coupler including: a first wall defining a central lumen; a plurality of projections extending outward from the first wall; a plurality of second walls, each second wall extending between two projections of the plurality of projections; and a plurality of slots, wherein each slot is defined by the first wall, one of the second walls, and two of the plurality of projections, wherein the slot is configured to receive a single spline of a basket.

Each projection of the plurality of projections may include a curved leading section with a greater radius of curvature than a curved trailing section.

The curved leading section may curve out and away from a distal face of the coupler.

Each projection of the plurality of projections may further include a side extending at a first angle (A5) to a line bisecting the slot.

Each second wall may define a through hole in communication with the slot, the through hole configured for securing the spline in the slot.

Each second wall may be recessed in a longitudinal direction and/or a radial direction.

A first end of the second wall and a first end of the first wall may be aligned.

A distal face of the distal coupler may define an opening in communication with the central lumen.

Each slot may be positioned opposite to another slot.

The distal face may be symmetrical.

Adjacent slots may be positioned at a second angle (A4).

The distal coupler may further include at least one rib extending from the first wall adjacent to the plurality of projections and the plurality of second walls.

The sides of each rib may be oriented to form a third angle (A3).

The first wall may define at least one through hole for securing the coupler to the shaft.

The at least one through hole may include a first through hole and a second through hole and wherein one or more of the at least one rib may be positioned between the first through hole and the second through hole.

The at least one through hole may be a hole or a slit.

One of the least one through hole may be positioned adjacent to a second end of the coupler.

The present disclosure also relates to a shaft of a catheter including a helical pattern cut into a wall of the shaft, wherein the helical pattern includes a helical backbone and interlocking elements oriented at an oblique angle to a longitudinal axis of the shaft, wherein the helical backbone includes alternating first and second sections, wherein a pair of the interlocking elements extend from opposite sides of each first section of the helical backbone, each interlocking element including a post and two arms oriented at an oblique angle relative to one another, the two arms including curved edges; and sides of each second section are complementary to a shape of interlocking elements extending from an adjacent turns of the helical backbone.

The present disclosure also relates to a shaft of a catheter including a helical pattern cut into a wall of the shaft, wherein the helical pattern includes a helical backbone and interlocking first and second elements oriented at an oblique angle to a longitudinal axis of the shaft, wherein the first and second elements have different shapes.

Each second element may define a slot configured to receive one of the first elements.

Each first element may include a stem and a rounded end with a diameter greater than a width of the stem, and the stem extends from a side of the helical backbone.

Each second element may include a pair of members.

The pair of members may define a closed end of the slot and an opening of the slot, wherein the opening may be sized to maintain the rounded end of the one of the first elements in the slot.

Each member of the pair of members may include a curved section with a radius of curvature complementary to the radius of curvature of the rounded end of the one of the first elements in the slot.

The second element may be symmetrical about a longitudinal line bisecting the slot.

The members of the pair of members may be mirror images of each other.

The shaft may be made of a polymeric material.

The shaft may be made of a metallic material.

The present disclosure also relates to a medical device including: a distal coupler as described herein, wherein the coupler is attached to a first shaft; and a basket coupled to the first shaft by the distal coupler, the basket including a plurality of splines, wherein each of the plurality of splines is secured to one of the plurality of slots of the distal coupler.

The present disclosure also relates to a medical device including: a shaft as described herein, the shaft including a first shaft; a distal coupler as described herein, wherein the distal coupler is attached to the first shaft; and a basket coupled to the first shaft by the distal coupler, the basket including a plurality of splines, wherein each of the plurality of splines is secured to one of the slots of the coupler.

The present disclosure also relates to a medical device, wherein the first shaft is a shaft as described herein, wherein the helical pattern may be located in a length of the wall of the first shaft positioned within the basket.

The distal coupler may be attached to the first shaft distal to the helical pattern.

The distal face of the distal coupler may be a distal face of the medical device.

The basket may further include one or more tethers, each tether may extend between adjacent splines of the plurality of splines.

The one or more tethers may be positioned in a distal region of the basket.

Each tether may be oriented circumferentially.

Each tether may be oriented with a first end positioned forward of a second end.

The tether may be made of a metallic material.

The basket may further include one or more electrodes for ablation and/or mapping.

Each spline may include at least one of the one or more electrodes.

The medical device may further include an expandable balloon positioned within the basket, the expandable balloon including: an outer surface; a distal end attached to the distal coupler; and a proximal end attached to a proximal coupler; wherein the expandable balloon is selectively expandable such that, when the expandable balloon is in an expanded state, the outer surface of the expandable balloon contacts an inner surface of the plurality of splines.

A first sensor may be positioned between an inner surface of a first spline of the plurality of splines and an outer surface of the balloon, wherein the first sensor may be configured to output a signal indicative of an expansion state of the expandable balloon.

The first sensor may be a first electrode, and a measured electrical response between the first electrode and a second electrode may be the signal indicative of the expansion state.

The first electrode may be an exposed end of a first wire affixed to an insulated portion of the first spline.

A high impedance path may be provided between the first electrode and the second electrode when the expandable balloon is in an expanded state, and a low impedance path may be provided between the first and second electrode when the expandable balloon is in an unexpanded state.

The second electrode may be an electrode located on a second spline of the plurality of splines, the second spline including: a body including a conductive material; and an insulating material covering portions of the body, wherein the electrode includes an exposed portion of the body.

A proximal end of the basket may be attached to a distal end of a second shaft.

The second electrode may be a ring electrode, located on the second shaft proximal to the basket.

The second electrode may be an exposed end of a second wire located at an inner surface of a second spline of the plurality of splines, wherein the exposed end of the second wire may be coupled to an insulated portion of a body of the second spline of the plurality of splines.

A low impedance path may be provided between the first electrode and the second electrode when the expandable balloon is in the expanded state, and a high impedance path may be provided between the first and second electrode when the expandable balloon is in the unexpanded state.

The second electrode may be affixed to an outer surface of the expandable balloon and aligned with the first electrode.

The present disclosure also relates to a medical device, wherein the sensor is a pressure sensor coupled to an insulated portion of an inner surface of the first spline of the plurality of splines, wherein an increasing pressure is provided between the pressure sensor and the outer surface of the expandable balloon when the balloon is inflating, and a decreasing pressure is provided between the pressure sensor and the outer surface of the balloon when the balloon is deflating.

The present disclosure also relates to a method of detecting balloon expansion of an expandable balloon of a medical device, the medical device having a basket including plurality of splines, the balloon positioned within the plurality of splines, the method including: measuring, by a sensor positioned between an inner surface of a first spline of the plurality of splines and an outer surface of the expandable balloon, a characteristic indicative of an expansion state of the expandable balloon; determining the expansion state of the expandable balloon based on the measured characteristic; and displaying the expansion state of the expandable balloon on a display.

The sensor may be a first electrode, and wherein the measured characteristic associated with the first electrode may be an impedance between the first electrode and a second electrode.

Determining the expansion state of the expandable balloon may include comparing the measured impedance to a first threshold, wherein the expandable balloon may be determined to be in an inflated state when the impedance is greater than the first threshold, and wherein the expandable balloon may be determined to be in an uninflated state when the impedance is less than the first threshold.

Determining the expansion state of the expandable balloon may further include comparing the measured impedance to a second threshold, wherein the expandable balloon may be determined to be in an overinflated state when the impedance is greater than the second threshold.

The second electrode may be located on the outer surface of the balloon aligned with the first electrode, wherein determining the expansion state of the expandable balloon may include comparing the measured impedance to a first threshold, wherein the expandable balloon may be determined to be in an inflated state when the impedance is less than the first threshold, and wherein the expandable balloon may be determined to be in an uninflated state when the impedance is greater than the first threshold.

The sensor may be a pressure sensor, and wherein the measured characteristic may be a pressure between the pressure sensor and the expandable balloon, wherein determining the expansion state of the expandable balloon may include comparing the measured pressure to a first threshold; wherein the balloon may be determined to be in an inflated state when the pressure is greater than the first threshold; and wherein the balloon may be determined to be in an uninflated state when the pressure is less than the first threshold.

The present disclosure also relates to a system for detecting balloon inflation, the system including: a medical device including: a basket assembly affixed to a distal end of a shaft, the basket assembly including a plurality of splines; an expandable balloon having an outer surface, wherein the balloon is positioned within the basket assembly, and wherein the expandable balloon is selectively expandable such that the outer surface of the expandable balloon is contactable with an inner surface of at least one of the plurality of splines; and a first sensor positioned between an inner surface of a first spline of the plurality of splines and the outer surface of the balloon, wherein the first sensor is configured to monitor a characteristic indicative of an expansion state of the expandable balloon; an electronic control unit configured to receive feedback indicative of the measured characteristic from the first sensor, wherein the electronic control unit determines the expansion state based on the feedback and generates an output; and a display that receives the output from the electronic control unit and displays the balloon expansion state.

The first sensor may be a first electrode.

The first electrode may be an exposed end of a first wire affixed to an insulated portion of the first spline.

The measured characteristic may be an impedance measured between the first electrode and a second electrode, wherein the electronic control unit may compare the measured impedance to a first threshold, and wherein the balloon may be determined to be in an inflated state when the impedance is greater than the first threshold, and wherein the balloon may be determined to be in an uninflated state when the impedance is less than the first threshold.

The second electrode may be an electrode located on a second spline of the plurality of splines, the second spline including: a first end coupled to the distal end of an outer shaft element; a second end coupled to the distal end of a shaft element positioned within the balloon; a body including a conductive material; and an insulating material covering portions of the body, wherein the second electrode includes an exposed portion of the body.

The second electrode may be a ring electrode located at the distal end of the shaft proximal to the basket assembly.

The second electrode may be an exposed end of a second wire located at an inner surface of a second spline of the plurality of splines, wherein the exposed end of the second wire may be coupled to an insulated portion of a body of the second spline.

The electronic control unit further compares the measured impedance to a second threshold, and wherein the balloon may be determined to be in an overinflated state when the impedance is greater than the second threshold.

The measured characteristic may be an impedance measured between the first electrode and a second electrode, wherein the second electrode may be affixed to an outer surface of the balloon and aligned with the first electrode, and wherein the electronic control unit may compare the measured impedance to a first threshold, and wherein the balloon may be determined to be in an inflated state when the impedance is less than the first threshold, and wherein the balloon may be determined to be in an uninflated state when the impedance is greater than the first threshold.

The first sensor may be a pressure sensor coupled to an insulated portion of an inner surface of the first spline, and the characteristic indicative of the balloon expansion state may be a measured pressure between the pressure sensor and the outer surface of the balloon, wherein the electronic control unit may compare the measured pressure to a first threshold, and wherein the balloon may be determined to be in an inflated state when the pressure is greater than the first threshold, and wherein the balloon may be determined to be in an uninflated state when the pressure is less than the first threshold.

The present disclosure also relates to a medical device including: a basket; a distal coupler attached to a distal end of the basket; a proximal coupler attached to a proximal end of the basket and to a catheter shaft, the proximal coupler defining a lumen; and a support shaft including: a distal end secured to the distal coupler; and a proximal end free floating in the lumen of the proximal coupler.

The present disclosure also relates to a medical device including: a distal coupler including: a basket securement region; a balloon region proximal to the basket securement region; and at least one magnetic sensor positioned along an outer surface of the distal coupler in a groove; a proximal coupler including: a balloon securement region; and a basket securement region proximal to the balloon securement region; and at least one magnetic sensor secured to an outer surface of the proximal coupler; a basket coupled to the basket securement regions of the distal and proximal couplers; and a balloon positioned in the basket, the balloon coupled to the balloon securement regions of the distal and proximal couplers.

The present disclosure also relates to a medical device including: a basket including a plurality of splines; a distal coupler including: a distal face; and a spline retention component configured to secure distal ends of the plurality of splines; wherein the plurality of splines positioned in the spline retention component are at a tangent to the distal face.

The present disclosure also relates to a basket including a plurality of splines, wherein a distal end of each of the plurality of splines is a barb including at least one projection.

The present disclosure also relates to a coupler including: a first securement component including slots, the slots including a first open end and a second open end; an annular section adjacent to the second open end, the annular section configured to hold barbed ends of basket splines; and an annular wall adjacent to the annular section.

The present disclosure also relates to a coupler including: a balloon securement region including: a plurality of ribs; and at least one magnetic sensor secured to a groove in an outer surface of the balloon securement region, the groove extending through at least some of the plurality of ribs; and a basket securement region longitudinally adjacent to the balloon securement region.

The present disclosure also relates to a shaft for a medical device including: a polymeric tube forming an inner layer of the shaft; a braid positioned on, and coextensive with, the polymeric tube; a coil positioned on a first end section of the braid; a first polymer with a first durometer hardness over the coil; a second polymer with a second durometer hardness over an intermediate section of the braid; and a third polymer with a third durometer hardness over a second end section of the braid.

The present disclosure also relates to a method of manufacturing a shaft for a catheter including: forming a braid around a polymeric tube; coiling a wire around a first end section of the braid; reflowing a first polymer with a first durometer hardness on the coiled wire; reflowing a second polymer with a second durometer hardness on an intermediate section of the braid; and reflowing a third polymer with a third durometer hardness on a proximal section of the braid.

The first polymer, the second polymer, and the third polymer may form an outer layer of the shaft.

The second durometer hardness may be less than the first durometer hardness and the first durometer hardness is less than the third durometer hardness.

The polymeric tube may be a tube of polytetrafluoroethylene (PTFE), the braid includes a plurality of metallic wires, the coil wire may be a metallic wire, the first polymer may be polyether block amid (PEBA), the second polymer may be polyether block amid (PEBA), and the third polymer may be polyether block amid (PEBA).

The plurality of metallic wires may include stainless steel and the coil wire may include stainless steel.

The first polymer, the second polymer, and the third polymer may be positioned within gaps defined by the braid and/or the coil of wire.

The first section has a first length, the second section has a second length, and the third section has a third length, wherein the second length may be greater than the first length and the third length; and/or the first length may be the smallest length.

The shaft may form a part of a medical device further including: an outer shaft coaxial with the shaft; a basket; a distal coupler securing a distal end of the basket to a distal end of the shaft; and a proximal coupler securing a proximal end of the basket to the outer shaft; wherein the basket is supported by the first section of the shaft.

The basket may be supported only by the first section of the shaft extending between the distal and proximal couplers.

### BRIEF DESCRIPTION OF DRAWINGS

FIG. 1 is a diagrammatic view of a system that includes a catheter with an expandable end assembly that may be utilized for mapping and/or ablation.

FIG. 2 is a schematic view of a catheter with a basket that may be utilized with the system shown in FIG. 1.

FIG. 3A is a cross-sectional view of a catheter with a basket-balloon assembly that may be utilized with the system shown in FIG. 1.

FIG. 3B is an isometric view of a basket that may be utilized for the basket-balloon assembly shown in FIG. 3A and/or with the system shown in FIG. 1.

FIG. 3C is an isometric view of a basket-balloon assembly that may be utilized with the system shown in FIG. 1.

FIG. 4A is an isometric view of a coupler configured to attach the distal end of the basket to a catheter shaft.

FIG. 4B is an isometric view of a coupler configured to attach the distal end of the basket to a catheter shaft.

FIG. 4C is a side view of the coupler shown in FIG. 4B.

FIG. 4D is an isometric view of a coupler configured to attach the distal end of the basket to a catheter shaft.

FIG. 4E is an isometric view of a coupler configured to attach the distal end of the basket to a catheter shaft.

FIG. 4F is an isometric view of a barb.

FIG. 4G is an isometric view of a barb.

FIG. 5 is a side view of the coupler shown in FIG. 4A.

FIG. 6 is a cross-sectional view of the coupler shown in FIG. 5 taken along line A-A.

FIG. 7 is an enlarged view of area B of the coupler shown in FIG. 5.

FIG. 8 is a view of the distal end of the coupler shown in FIG. 5.

FIG. 9 is a view of the proximal end of the coupler shown in FIG. 5.

FIG. 10 is a first isometric view of a proximal coupler configured to attach the proximal end of the basket to a catheter shaft.

FIG. 11 is a second isometric view of the proximal coupler shown in FIG. 10.

FIG. 12 is a cross-sectional view of the proximal coupler shown in FIG. 10.

FIG. 13 is an end view of the proximal coupler shown in FIG. 10.

FIGS. 14A and 14B are isometric views showing basket splines coupled to the proximal coupler shown in FIG. 10.

FIG. 15 is an isometric view of a retention ring configured to be attached to the proximal coupler shown in FIG. 10.

FIG. 16 is an isometric view of a basket secured to a catheter by the proximal coupler, shown in FIG. 10.

FIG. 17 is an isometric view of a proximal coupler configured to attach the proximal end of the basket to a catheter shaft.

FIG. 18 is a cross-sectional view of the proximal coupler shown in FIG. 17.

FIG. 19 is an isometric view of the retention ring and the coupler component of the proximal coupler shown in FIG. 17.

FIG. 20A is an isometric view of the retention ring of the proximal coupler shown in FIG. 17 and FIG. 20B is an end view of the retention ring of the proximal coupler shown in FIG. 17.

FIG. 21 is an isometric view of a catheter shaft with a non-helical cut pattern.

FIG. 22 is a side view of a catheter shaft with a helical cut pattern.

FIG. 23 is a side view illustrating the catheter shaft illustrated in FIG. 22 with a bend.

FIG. 24 is a flat plan view of a portion of the helical cut pattern oriented at a non-helical orientation to illustrate details of the helical cut pattern shown in FIG. 22.

FIG. 25 is an isometric view of a portion of the helical cut pattern oriented at a non-helical orientation to illustrate details of the helical cut pattern shown in FIG. 22.

FIG. 26 is a flat plan view of a portion of the helical cut pattern oriented at a non-helical orientation to illustrate details of the helical cut pattern shown in FIG. 22.

FIG. 27 is a flat plan view of a portion of the helical cut pattern oriented at a non-helical orientation to illustrate details of the helical cut pattern shown in FIG. 22.

FIG. 28 is a side view of a catheter shaft with a helical cut pattern.

FIG. 29 is a side view illustrating the catheter shaft illustrated in FIG. 28 with a bend.

FIG. 30 is an isometric view of a portion of the helical cut pattern oriented at a non-helical orientation to illustrate details of the helical cut pattern shown in FIG. 28.

FIG. 31 is a flat plan view of an element of the helical cut pattern shown in FIG. 28.

FIG. 32 is a flat plan view of a portion of the helical cut pattern oriented at a non-helical orientation to illustrate details of the helical cut pattern shown in FIG. 28.

FIG. 33 is a flat plan view of a portion of the helical cut pattern oriented at a non-helical orientation to illustrate details of the helical cut pattern shown in FIG. 28.

FIG. 34 is a side view of a balloon-basket assembly.

FIG. 35 is a view of the distal end of the balloon-basket assembly shown in FIG. 34.

FIG. 36 is a side view of a balloon-basket assembly.

FIG. 37 is an isometric view of one embodiment of a basket-balloon assembly of a catheter that may be used with the system shown in FIG. 1 where the balloon is in an expanded state.

FIG. 38 is an isometric view of another embodiment of a basket that may be used in a basket-balloon end assembly of a catheter that may be used with the system shown in FIG. 1.

FIG. 39A and FIG. 39B are side views of a portion of a basket-balloon assembly.

FIG. 40A and FIG. 40B are side views of a portion of a basket-balloon assembly.

FIG. 41A and FIG. 41B are side views of a portion of a basket-balloon assembly.

FIG. 42 is a flowchart illustrating a method of determining the expansion state of the balloon.

FIG. 43 is an isometric view of a catheter shaft.

FIG. 44 is a cross-sectional view of a section of the catheter shaft.

FIG. 45 is a cross-sectional view of a section of the catheter shaft.

FIG. 46 is an isometric view of a medical device that includes a basket-balloon assembly.

FIG. 47 is a flow chart of a method of manufacturing a catheter shaft.

FIG. 48 is an isometric view illustrating features of the distal end of a basket secured to a coupler.

FIG. 49 is an isometric view illustrating features of the distal end of a basket-balloon assembly secured to a coupler.

FIG. 50 is a cross-sectional view illustrating features of a coupler with an offset sensor.

FIG. 51 is an isometric view illustrating a distal coupler.

FIG. 52 is an isometric view illustrating a distal coupler.

FIG. 53 an isometric view illustrating features of a proximal coupler.

FIG. 54 is an isometric view illustrating features of a proximal coupler.

FIG. 55 is an isometric view illustrating features of a proximal coupler.

FIG. 56 is an isometric view illustrating features of a proximal coupler.

FIG. 57 is a cross-sectional view illustrating features of a proximal coupler.

FIG. 58 is an isometric view illustrating features of a proximal coupler.

FIGS. 59 and 60 are isometric views illustrating features of a proximal coupler.

FIG. 61 is an isometric view illustrating features of a proximal coupler.

FIG. 62 is a cross-sectional view illustrating features of a proximal coupler.

### DETAILED DESCRIPTION

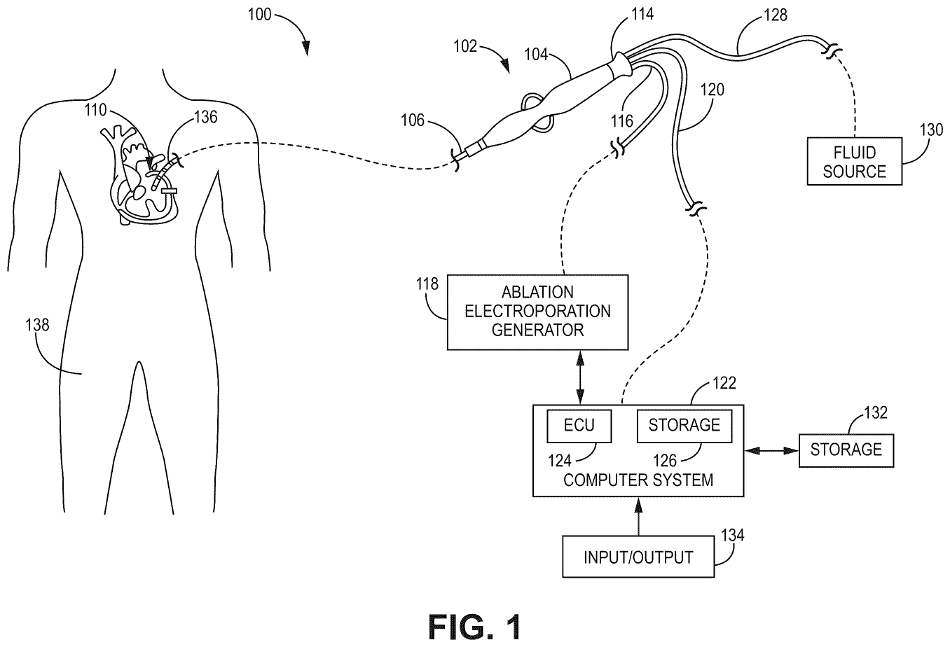

FIG. 1 is an isometric view of a system 100 utilized in a medical procedure with respect to patient 138. The system 100 may include a medical device such as a catheter 102, a mapping and/or ablation system 118 compatible with the catheter 102, a computer system 122 including a display 132 and an input/output device 134, and a fluid source 130.

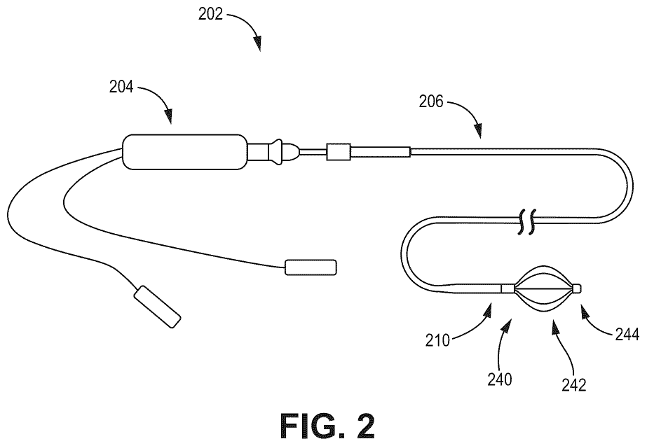

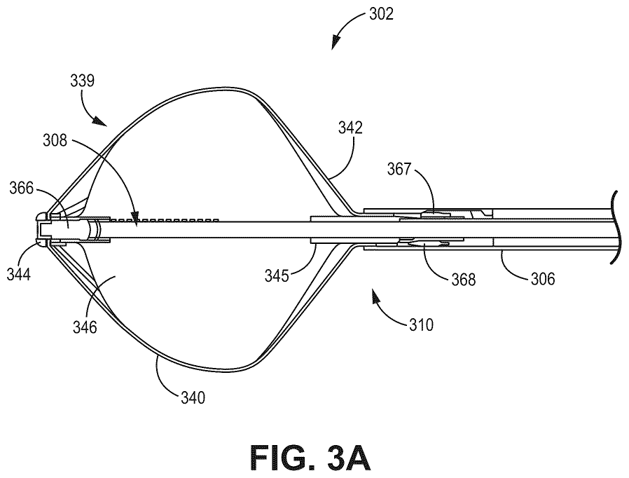

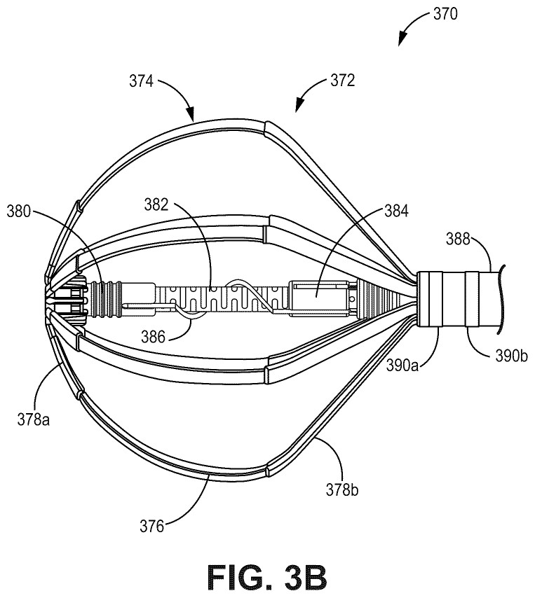

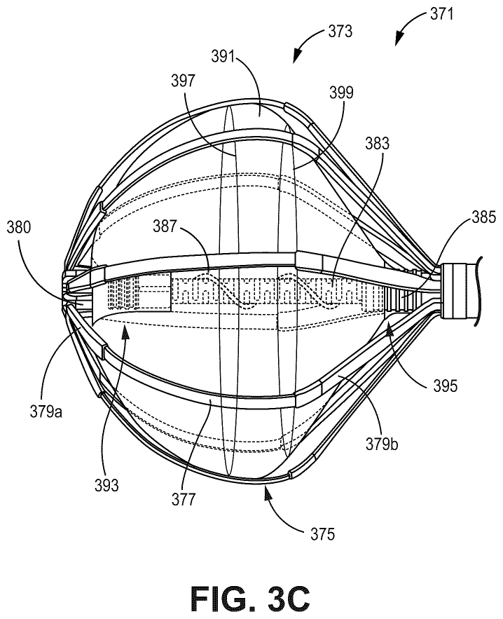

The catheter 102 of system 100 includes a handle 104, at least one shaft, such as shaft 106, extending distally from the handle 104, a cable connector 114, and an expandable end assembly positioned at the distal end 110 of the catheter 102. The expandable end assembly may be a basket (shown in FIG. 2). The expandable end assembly may be a balloon-basket assembly comprising a balloon positioned within a basket (shown for example in FIGS. 3A and 3C). As discussed below in greater detail, the expandable end assembly may be coupled to the medical device by at least one coupler.

The catheter 102 may be a cardiac mapping device. The catheter 102 may be a therapy delivery device configured to deliver ablation therapy wherein at least a portion, e.g., portion 136, of the catheter 102 is located within a patient 138. Exemplary ablation therapies include radiofrequency (RF) ablation and/or electroporation ablation.

The mapping and/or ablation system 118 may be connected to catheter 102 via cable connector 114 and cable 116 to deliver therapy. The mapping and/or ablation system 118 may include a generator capable of providing energy via cable 116 and one or more wires included within the shaft 106 to one or more electrodes located on the expandable end assembly. The electrodes may be utilized for cardiac mapping and/or ablation. The mapping and/or ablation system 118 may be configured to provide radio frequency (RF) energy to a catheter 102 configured to perform RF ablation. The mapping and/or ablation system 118 may be configured to provide short-duration direct current (DC) pulses to perform electroporation ablation (i.e., pulsed field ablation). The mapping and/or ablation system 118 may be coupled to computer system 122. For example, computer system 122 may provide instructions to mapping and/or ablation system 118 (e.g., definition of electrical pulses, selection of electrodes to receive pulses, etc.).

The system 100 may include a fluid source 130 connected to catheter 102 via cable 128 and cable connector 114 to deliver fluid to the catheter 102. The fluid source may provide inflation media to the interior of the balloon to inflate the balloon. The amount of fluid delivered may be selectively adjusted depending on the expansion state of the balloon and the desired expansion state.

The computer system 122 may connected to catheter 102 via cable 120 and cable connector 114. As shown in FIG. 1, the computer system 122 may include an electronic control unit (ECU) 124 and computer readable medium or storage unit 126. The ECU 124 may include a processor configured to execute instructions stored by the computer readable medium 126 to implement one or more functions described herein. For example, the computer system 122 may be configured to receive one or more signals generated by sensors located at the distal end 110 of the catheter 102 (e.g., sensors located on the expandable end assembly, on the shaft, etc.). The feedback received may be utilized to determine the location of the catheter 102 within the patient 138 (e.g., localization/navigation), to assess therapy status (e.g., lesion assessment, tissue contact, etc.), map electrophysiology characteristics of the heart (e.g., cardiac mapping), and/or to determine operational status of the catheter (e.g., balloon expansion status, electrode status, etc.). The computer system 122 may generate one or more outputs directed to these functions that are displayed on the display 132.

The expandable end assembly, located at the distal end 110 of the catheter, may be inserted into a body region of interest during a medical procedure, for example within the vasculature of the patient, and movement of the catheter 102 may be facilitated by one or more controls located on handle 104. Typically, the expandable end assembly is operable between a collapsed state and an expanded state. In the collapsed state, the expandable end assembly is navigable within the vasculature of the patient due to the smaller overall diameter of the expandable end assembly. Upon reaching a desired position, the expandable end assembly may be expanded.

FIG. 2 is an isometric view of a medical device, such as a catheter 202, that may be utilized in a medical procedure with respect to patient 138. As shown in FIG. 2, the medical device, such as catheter 202, may include a handle 204, at least one shaft 206, an expandable end assembly, in the form of a basket 240 with a plurality of splines 242, positioned at the distal end 210 of the catheter 202, and a least one coupler configured to secure the basket 240 to the catheter 202, such as distal coupler 244.

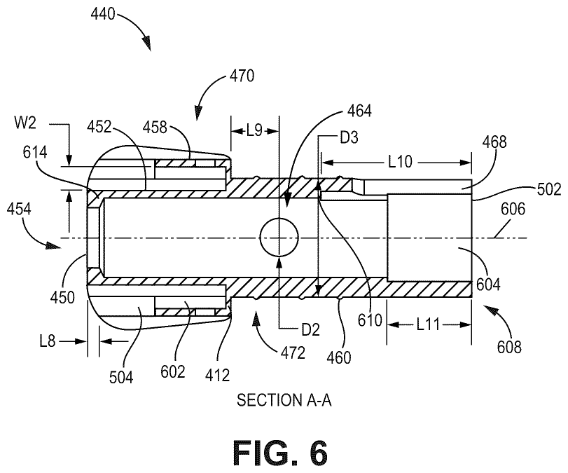

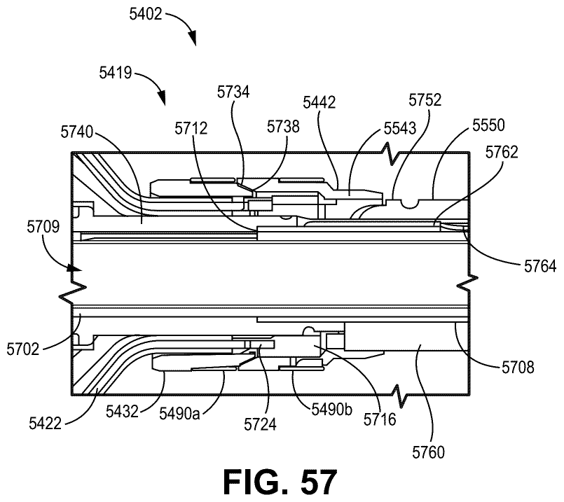

The at least one shaft 206 may include an inner shaft, an outer shaft, and/or a support shaft. For example, the catheter 202 may include an inner shaft positioned within an outer shaft, as illustrated in FIG. 3A. As another example, the catheter may include an outer shaft and a support shaft configured to support an end assembly, as illustrated in FIGS. 3B and 3C. The catheter 202 may include a proximal coupler and a distal coupler. As used in this application “proximal” is closer to the user and “distal” is farther from the user. A coupler may be a single component or unitary (see e.g., FIG. 6) or may be formed by a plurality of components (see e.g., FIG. 57). A coupler may modular. For example, the coupler may include a male section received by a female section. A coupler with a male/female connection may further include a poka yoke alignment/retention feature to orient the male/female sections. A coupler may include at least one securement region. A securement region may be configured to secure a basket and/or a balloon of an expandable end assembly. A coupler may include a basket securement region positioned longitudinally adjacent to a balloon securement region. The distal coupler 244 may be configured to secure components of a catheter together. For example, the distal coupler may couple the splines 242 of the basket 240 to a shaft 206, such as an inner shaft as illustrated in FIG. 3A, or to a basket support shaft as illustrated in FIGS. 3B and 3C. Other features of a coupler are discussed below in greater detail.

As discussed above, the expandable end assembly, such as basket 240, may include at least one electrode for ablation and/or mapping. The electrode may be carried by the basket. For example, the spline may be formed of a conductive material and the electrode may be an exposed region of a spline (as shown for example in FIG. 3B). The electrode may be attached to a spline. For example, the electrode may be a ring electrode. For purposes of discussion, the figures illustrate baskets with eight basket splines configured to be oriented generally parallel to the longitudinal axis of the end assembly when the basket is in an expanded state (see e.g., FIG. 3B). However, baskets with more or less than eight basket splines and/or basket splines oriented non-parallel to the longitudinal axis of the end assembly, e.g., a petal shape, when the basket is in an expanded state may be utilized with medical devices having one or more features and/or components disclosed herein.

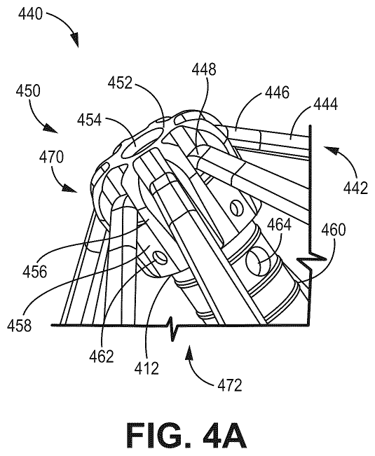

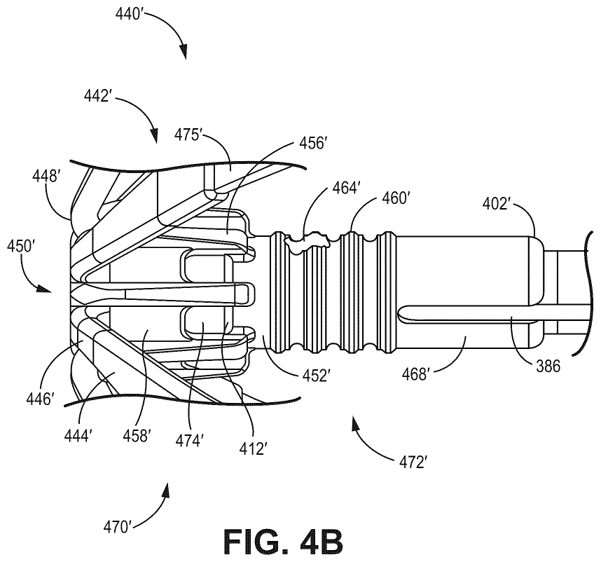

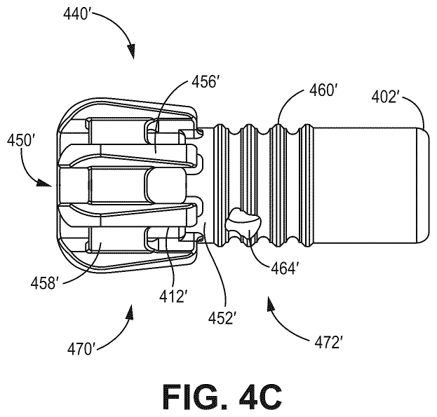

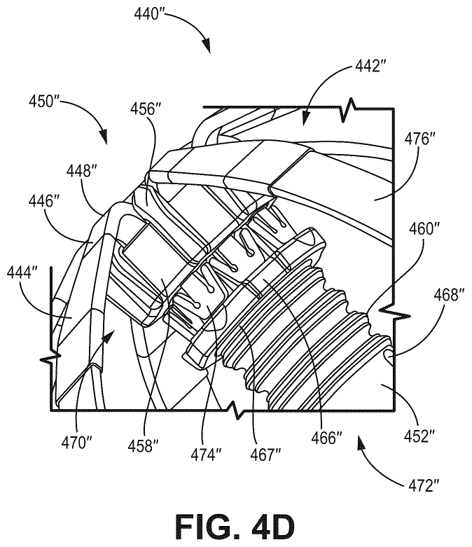

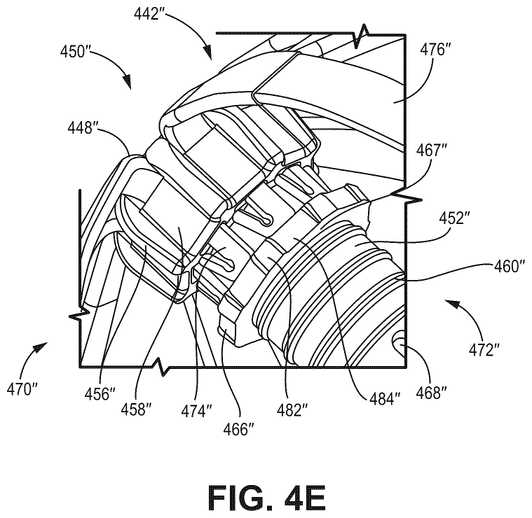

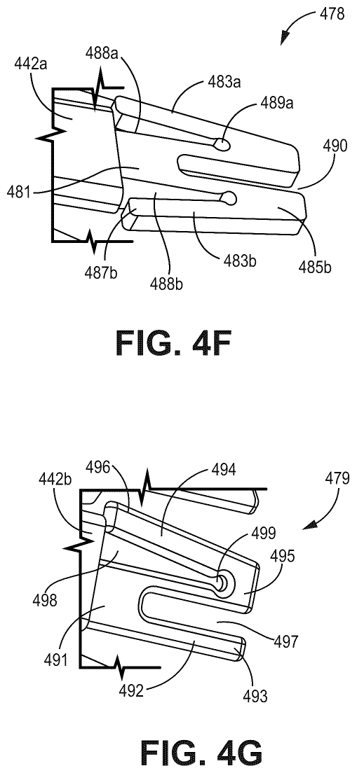

The end regions of exemplary medical devices provided in FIGS. 3A-3B generally illustrate components and features that may be provided. Additional details of the components and features are illustrated in FIGS. 4A-41, 43-46, and 48-63.

FIG. 3A is a cross-sectional view of an end region of a medical device, such as a catheter 302, that may be utilized in a medical procedure with respect to patient 138. As shown in FIG. 3A, the medical device, such as catheter 302, may include an outer shaft 306, an inner shaft 308 positioned inside the outer shaft 306, and an expandable end assembly 339 located at the distal end 310 of the catheter 302. Like the catheter 202 shown in FIG. 2, the end assembly 339 includes a basket 340 comprising a plurality of splines 342. The catheter 302 may include a distal coupler 344 configured to secure the basket 340 to the inner shaft 308 and a proximal coupler 345 configured to secure the end assembly 339 to the inner shaft 308 and/or the outer shaft 306. Like basket 240, basket 340 may include at least one electrode for ablation and/or mapping.

The end assembly 339 illustrated in FIG. 3A further includes an expandable balloon 346 positioned inside the basket 340. The balloon 346 may be secured to the distal coupler 344 and to the proximal coupler 345. The balloon 346 may be made from an insulative material. For example, the balloon material may be one or more of nylon, polyamine, ethylene-vinyl acetate, polyvinyl chloride (PVC), olefin copolymers or homopolymers, polyethylenes, highly irradiated linear low density polyethylene (LDPE), polyurethanes, crosslinked low density polyethylenes (PETs), acrylonitrile polymers and copolymers, or acrylonitrile blends and ionomer resins. As discussed above, fluid from the fluid source 130 may be provided to the balloon 346 to inflate the balloon within the interior of the basket.

The medical device may further include at least one magnetic sensor. The at least one magnetic sensor may be utilized for navigation (e.g., determining a position and/or orientation of the medical device within a patient).

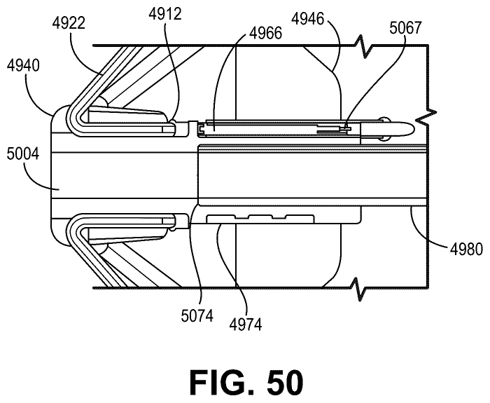

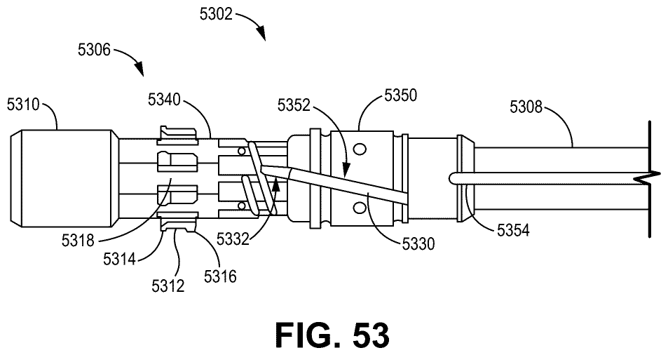

The magnetic sensor may be a solid core magnetic sensor or a hollow core magnetic sensor. The magnetic sensor may be configured to sense an electromagnetic field. At least one magnetic sensor may be positioned adjacent to the distal end of the expandable end assembly (a distal magnetic sensor) and at least one magnetic sensor may be positioned adjacent to the proximal end of the expandable end assembly (a proximal magnetic sensor). The longitudinal axis of the magnetic sensor may be parallel to, at an oblique angle to, and/or offset from the longitudinal axis of the catheter (see FIGS. 3A, 50, and 51). As discussed below in greater detail, the distal magnetic sensor may be coupled to the distal coupler and the proximal magnetic sensor(s) may be coupled to the proximal coupler. Sensor wires coupled to the magnetic sensor may include a protective layer. For example, the sensor wires may be surrounded by a protective tubing. The magnetic sensor may have sensor wires extending from a single end (hereinafter the wire end). The wire end may be oriented proximally (see e.g., FIG. 50), referred to as a proximally oriented magnetic sensor) or distally (see e.g., FIG. 53), referred to as a distally oriented magnetic sensor.

Returning to FIG. 3A, the catheter 302 includes a magnetic sensor 366 positioned adjacent to the distal end of the end assembly 339 and a plurality of magnetic sensors 367, 368 positioned adjacent to the proximal end of the end assembly 339. In this example, the magnetic sensor 366 is positioned within the distal coupler 344 and is coaxial with the inner shaft 308 and the longitudinal axis of the catheter 302 and the plurality of magnetic sensors 367, 368, positioned outside of the inner shaft 308, are offset from the longitudinal axis of the catheter 302.

FIG. 3B is an isometric view of the end region of a medical device, such as a catheter 370, that may be utilized in a medical procedure with respect to patient 138. The catheter 370 includes a basket 372 secured to a distal coupler 380 and to a proximal coupler 384, a sensor (not shown) positioned in the distal coupler 380 and coupled to at least one sensor wire 386, a support shaft 382 positioned within the basket 372 and extending between the distal coupler 380 and the proximal coupler 384, a shaft 388 extending proximally from the proximal coupler 384, and one or more shaft electrodes 390a, 390b (collectively electrodes 390).

One or more electrodes, such as electrodes 390, may be utilized for navigation, e.g., impedance tracking of the medical device (hereinafter navigation electrodes). Navigation electrodes may be coupled to the shaft, such as shaft 388, to the proximal coupler, and/or to the distal coupler. A method of navigating the medical device may utilize information from the at least one magnetic sensor and/or from the navigation electrodes.

A balloon may be positioned inside the basket 372, as shown in FIG. 3A. In this example, the plurality of splines 374 is eight splines. Like baskets 240, 340, basket 372 may include at least one electrode for ablation and/or mapping. In this example, the splines 374 are formed of a conductive material and the electrodes 376 are the exposed regions of the splines 374. Although FIG. 3B illustrates a single conductive region (electrode) 376 on each spline 374, a spline may have more than one conductive region (electrode). The conductive material may be nitinol. As shown, the insulative regions 378a, 378b (collectively insulative regions 378) are positioned on both sides of the electrodes. The insulative regions 378 may overmolded onto the spline. The insulative region may be a polyether block amide (PEBA). The PEBA material may have a durometer hardness of 72D. A spline with overmolded insulative regions may have a variable width. For example, spline regions configured to be overmolded with an insulative material may have a smaller width than spline regions configured to be conductive regions (electrodes).

The coupler may include at least one securement region. A securement region may be configured to secure a basket and/or a balloon of the expandable end assembly to the coupler. A coupler may include a basket securement region positioned longitudinally adjacent to a balloon securement region. As shown in FIG. 3B, the distal end of the basket 372 is secured to the distal coupler 380 and the proximal end of the basket 372 is secured to the proximal coupler 384. For catheters that include a balloon positioned within the basket, the distal coupler 380 may be secured to the distal end of the balloon and the proximal coupler 384 may be secured to the proximal end of the balloon, as discussed below in greater detail.

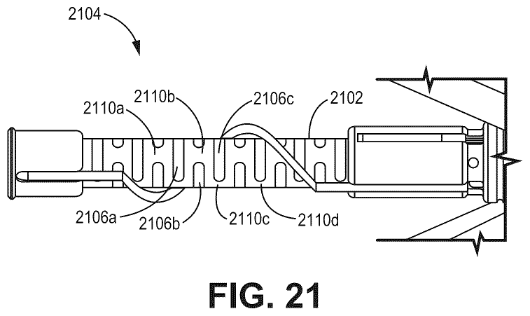

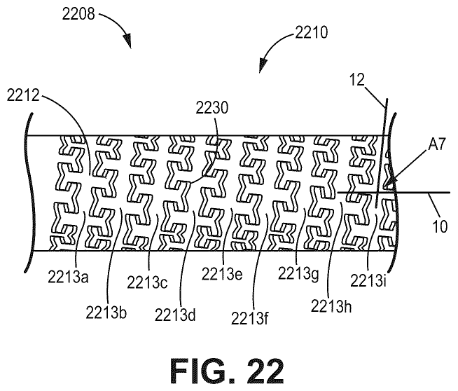

In contrast to the catheter illustrated in FIG. 3A, the distal coupler 380 is secured to a distal end of the support shaft 382 instead of to an inner shaft 308. The catheter 370 may lack an inner shaft positioned within shaft 388. A catheter without an inner shaft may have better maneuverability than a catheter with an inner shaft. The proximal end of the support shaft 382 may be secured to the proximal coupler 384. For example, as illustrated in FIG. 3B, the distal end of the support shaft 382 is positioned within a lumen defined by the distal coupler 380 and the proximal end of the support shaft 382 is positioned within a lumen defined by the proximal coupler 384. The distal end of the support shaft 382 may be secured to the distal coupler 380 and the proximal end of the support shaft 382 may be free floating in the lumen of the proximal coupler 384, as discussed below. The support shaft 382 may have a longitudinal length less than the longitudinal length of the basket 372. The support shaft 382 may include a cut pattern. The cut pattern may be non-helical, as illustrated in FIG. 21, or helical, as illustrated for example in FIG. 22. Examples of a cut pattern that may be applied to the support shaft 382 are discussed below in greater detail.

As shown in FIG. 3B, the sensor wires 386 extending from a sensor positioned in the distal coupler 380 may be tacked to the distal coupler 380 and/or to the proximal coupler 384. For example, the sensor wires 386 may extend along an outer surface of the distal coupler 380 and along an outer surface of the proximal coupler 384. The sensor wires 386 may include a strain relief mechanism. For example, the sensor wires 386 may be free floating—not tacked to the support shaft 382. As shown in FIG. 3B, the sensor wires 386 extend helically in a free-floating configuration around the support shaft 382.

FIG. 3C is an isometric view of the end region of a medical device, such as a catheter 371, that may be utilized in a medical procedure with respect to patient 138. The catheter 371 includes a basket 373 with a distal end secured to a distal coupler 381 and a proximal end secured to a proximal coupler 385, a balloon 391 positioned inside the basket 373, a sensor (not shown) positioned in the distal coupler 381 and coupled to at least one sensor wire 387, a support shaft 383 positioned within the balloon 391 and extending between the distal coupler 381 and the proximal coupler 385. Like the catheter illustrated in FIG. 3B, the plurality of splines 374 is eight splines. As discussed above, the basket 373 may include at least one electrode for ablation and/or mapping. Like basket 372 shown in FIG. 3B, the splines 375 are formed of a conductive material and the electrodes 377 are the exposed regions of the splines 375. Insulative regions 379a, 379b (collectively insulative regions 379) are positioned on both sides of the electrodes. Although FIG. 3C illustrates a single conductive region (electrode) 377 on each spline 375, a spline may have more than one electrode. The distal end of the support shaft 383 may be secured to the distal coupler 381 and the proximal end of the support shaft 383 may be free floating in the lumen of the proximal coupler 385, as discussed below. The support shaft 382 may include a cut pattern.

The balloon 391 may be secured to the distal coupler 381 and the proximal coupler 385. One end of the balloon may be inverted, both ends of the balloon may be inverted, or neither end of the balloon is inverted. For the catheter 371 shown in FIG. 3C, the distal end 393 of the balloon 391 is inverted such that the outer surface of the balloon 391 is coupled to the distal coupler 381 and the proximal end 395 of the balloon is not inverted. As illustrated in FIG. 3C, the balloon in an expanded state may have a first diameter 399 at a first longitudinal position, adjacent to a distal end of the insulative region 379b in this example, and a second diameter 397 at a second longitudinal position distal to the first longitudinal position where the second diameter 397 is greater than the first diameter 399.

End assemblies that include a balloon positioned inside a basket may be configured to have different performance parameters. Different performance outcomes may be provided by utilizing balloons with different unexpanded diameters, different expanded shapes, and/or different expanded diameter(s). For example, a smaller diameter balloon, configured to be inflated to the same diameter as a larger diameter balloon, has significantly less material than the larger balloon. Thus, a basket-balloon end assembly that includes a smaller diameter balloon may have freer movement through an introducer compared to a basket-balloon end assembly that includes a larger diameter balloon. Another advantage of a balloon with a small diameter is that less material may be required to manufacture the balloon, e.g., by blow molding. An advantage of a larger diameter balloon is that it may require less pressure to inflate compared to a smaller diameter balloon. However, a balloon with a larger diameter has significantly more material which may restrict spline motion if the material of the deflated balloon becomes positioned between basket splines and/or bunched under the basket splines.

As discussed above, the basket may be in a collapsed state when the catheter is advanced through an introducer. The configuration of the distal coupler may affect the handling of the medical device. For example, a typical distal coupler may restrict movement of the basket splines so that the splines act like a semi-rigid tube that cannot bend easily around corners and/or through a deflected sheath/introducer resulting in reduction of sheath shape. Due to the restriction in spline movement, additional force may be required to advance the catheter and/or reduces deflection of the introducer. Application of additional force may require a user to utilize both hands, with one hand holding the introducer as the other hand advances the catheter through the introducer. Additionally, the application of additional force may increase the risk of damage to the introducer and/or to the catheter.

The distal coupler features illustrated in FIGS. 4A-4E and FIGS. 48-50) may address one or more of these drawbacks. For example, utilizing a distal coupler as disclosed herein to secure the distal end of a basket to a shaft may allow the user to use one hand to manipulate the introducer and catheter, leaving the other hand free to do something else. The introducer may be held in the palm of the user's hand while the thumb and pointer finger of the hand are used to advance the catheter. As another example, utilizing a distal coupler as disclosed herein to secure the distal end of a basket to a shaft may improve performance. A catheter utilizing a distal coupler as disclosed herein has improved steerability. For example, in some embodiments, splines coupled to a distal coupler as disclosed herein have an additional degree of freedom because the splines can extend past the distal tip of the catheter as the catheter is being advanced through a deflected introducer, which may increase introducer deflection. A catheter utilizing distal coupler as disclosed herein may be inserted into an introducer with at least one bend—in other words, the introducer does not need to be straightened before inserting the catheter.

FIGS. 4A-4E and FIGS. 48-52 illustrate different features of a distal coupler. One benefit of the disclosed distal couplers is that the splines may extend beyond the distal face of the distal coupler as the catheter is advanced through an introducer. For example, as shown in FIGS. 4A-E the distal coupler lacks a feature, such as a stop or a cap, that may restrict or prevent movement of the splines beyond the distal face (hereinafter referred to as a capless distal coupler). Movement of the splines beyond the distal face of a capless distal coupler provides an extra degree of freedom to the splines. Movement of the splines beyond the distal face of the distal coupler may improve steerability of the catheter. For example, movement of the splines beyond the distal face of the coupler may reduce the loss of introducer deflection and/or the amount of force needed to advance the catheter through an introducer. Another benefit of a capless distal coupler is that the end assembly is shorter. Another benefit of the distal couplers illustrated in FIGS. 4A-4E, is that the distal coupler is a single piece construction. For example, the distal coupler may be molded.

Turning to FIG. 4A, the distal coupler 440 includes a distal face 450 with an end opening 454 defined by a first wall 452 that extends proximally from the distal face 450 to the proximal end of the coupler (not shown in this view), a first region 470 extending proximally from the distal face 450 and a second region 472 extending proximally from the first region to the proximal end. The distal face 450 of the distal coupler 440 may also the distal face of the catheter. As shown, the proximal end of the first region 470 may be blunt and the first region 470 may have a greater diameter than the second region 472.

The first region 470 includes a basket securement assembly configured to attach the basket splines 442 to the distal coupler 440. The basket securement assembly may form a proximal end of the first region 470. The basket securement assembly includes a plurality of slots configured to receive the basket splines 442, a plurality of projections 456 that extend outward from the first wall 452 a plurality of second walls 458 positioned between adjacent projections 456, and at least one wall 412 that extends from the first wall 452 (best shown in FIG. 6). This arrangement may assist in maintaining the axial positions of the splines 442. Additionally, this arrangement may simplify the attachment of the of the basket to the distal coupler 440. As illustrated, each slot may be defined by the first wall 452, the wall 412, a pair of projections 456, and one second wall 458. The slots may be blind holes (best shown in FIG. 6). The slots of the basket securement assembly may provide path for the splines 442 to move beyond the distal face 450 of the distal coupler 440. As shown in the figures, the slots are orientated parallel to the longitudinal axis of the distal coupler 440. However, the slots may be oriented at a non-oblique angle to the longitudinal axis of the distal coupler. Additionally, as discussed above, the couplers may be configured to secure more or less than eight basket splines by adjusting the number of slots.

The projections 456 may also provide protection to the basket during insertion/withdrawal through the introducer. For example, the projections 456 may provide a space between the inner diameter of the introducer and the basket. For baskets with one or more coated regions on the splines, such as spline section 444, this space may protect the coated regions during insertion/withdrawal of the basket through the introducer.

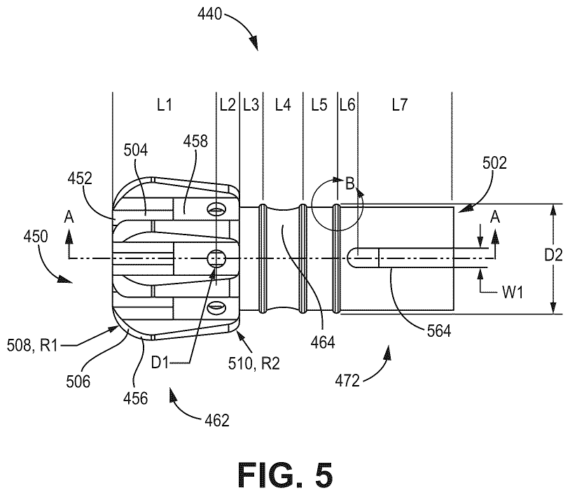

The second wall 458 may be recessed. For example, the second wall 458 may be radially recessed relative to the pair of projections 456. Recessed second walls 458 may provide a space sized to accommodate the splines 442 when the basket is in a collapsed state (not shown in FIG. 4A). For example, an inner surface of a spline 442 may be positioned adjacent to and/or contact the second wall when the basket is in a collapsed state. As another example, the second wall 458 may be longitudinally recessed relative to the distal face 450—in other words, a distal end of the second wall 458 is positioned a longitudinal distance away from the distal face 450. The second wall 458 may include a through hole 462 configured to secure the spline 442 to the distal coupler 440. For example, in some embodiments, an adhesive may be deposited into the through hole 462. The through hole 462 may be circular with a diameter (such as diameter D1 in FIG. 5).

As illustrated in FIG. 4A, the second region 472 may include at least one rib 460 extending from the first wall 452 and at least one through hole 464. The through hole 464 may be a mating feature for securing the distal coupler 440 to a shaft, such as an inner shaft 308 or a basket support shaft. For example, an adhesive may be deposited into the through hole 464. As another example, the through hole 464 may be utilized for welding. As illustrated in FIG. 4A, the distal coupler 440 has three ribs 460 extending circumferentially. The three ribs 460 are oriented circumferentially and may be described as circumferential ribs. The ribs 460 may provide a mechanical retention of a balloon secured to the distal coupler 440. The ribs 460 may provide an increased surface area for attachment of a balloon to the distal coupler 440. An increased surface area may improve the bond between the balloon and the distal coupler 440. The balloon may be attached to the distal coupler 440 by adhesive and/or laser bonding.

The second region 472 of the distal coupler 440 may have a smooth outer surface because it lacks ribs. For example, instead of ribs the section of the second region 472 may include overmolded portion formed of a material with a durometer hardness similar to the durometer hardness of the balloon material for bonding the balloon to the distal coupler. The material forming the distal coupler may be stiffer and/or harder than the material used for the overmolded portion. A laser may be utilized to bond the balloon to the overmolded portion of the distal coupler.

Turning to FIGS. 4B and 4C, features of a distal coupler 440′ is shown. Like the distal coupler 440 shown in FIG. 4A, the distal coupler 440′ includes a distal face 450′ with an end opening (not visible in this view) defined by a first wall 452′ that extends away from the distal face 450′, a first region 470′ that extends proximally from the distal face 450′ and a second region 472′ that extends proximally from the first region 470′ to the proximal end 402′. One difference between the distal coupler 440′ and the distal coupler 440 shown in FIG. 4A is that the distal coupler 440′ is shorter than distal coupler 440. For example, the first region 470′ is shorter (note difference in the positions of the basket splines relative to the distal face). This provides the end assembly with a shorter tip distal to the splines. The distal coupler may include a passage for a sensor wire coupled to a sensor positioned in the distal coupler, as discussed above. For example, in the view provided in FIG. 4B, a slot 468′, provided in the second region 472′, accommodates the sensor wire 386 (the slot is not visible in view provided in FIG. 4A).

Like the first region 470 shown in FIG. 4A, the first region 470′ has a greater diameter than the second region 472′ and includes a basket securement assembly that is configured to attach the basket splines 442′ to the distal coupler 440′, that forms a proximal end of the first region 470′, and that includes a plurality of slots configured to receive the basket splines 442′, a plurality of projections 456′ extending outward from the first wall 452′, a plurality of second walls 458′ with each second wall 458′ positioned between adjacent projections 456′, and at least one wall 412′. The distal ends of the second walls 458′ are positioned closer to the distal face 450′ compared to the distal ends of the second walls 458 of the distal coupler 440 shown in FIG. 4A. Additionally, the at least one wall 412′ is a plurality of walls 412′ positioned distal to the proximal ends of the projections, instead of being coextensive with the proximal ends of the projections, as shown in FIG. 4A. The distal end of the second wall 458′ may be distal to wall 412′. Each slot is defined by the first wall 452′, a pair of projections 456′, one second wall 458′, and one wall 412′ extending between adjacent projections and positioned distal to the proximal ends of the projections 456′. As discussed above, the projections 456′ may provide protection to the basket during insertion/withdrawal through the introducer. When basket splines 442′ are inserted into the slots, the splines 442′ are oriented at a tangent to the distal face 450′, in contrast to the basket splines 442 illustrated in FIG. 4A. Additionally, the second walls 458′ do not define a through hole. Rather, the proximal ends (trailing edges) of the second walls 458′ are distal to the walls 412′ so that the distal end 474′ of the basket spline 442′ is exposed/visible. One benefit of this arrangement is that the splines 442′ may be coupled to the distal coupler 440′ utilizing a mechanical mechanism instead of, or in addition to, adhesive. Although as shown in FIG. 4B, the proximal ends of the second walls 458′ are curved, the proximal ends of the second walls 458′ may be straight.

Like the second region 472 shown in FIG. 4A, the second region 472′ includes at least one rib 460′ extending from the first wall 452′ and a through hole 464′. As discussed above, the at least one rib 460″ may provide an increased surface area for attachment of a balloon to the distal coupler 440″. In contrast to the second region 472 shown in FIG. 4A, the second region 472′ includes four ribs 460′ and the first wall 452′ is concave between adjacent ribs 460′ rather than flat. The addition of concave curves between the ribs 460′ provides an increased surface area for attaching a balloon to the distal coupler 440′ compared to flat surfaces between adjacent ribs. Another difference between distal coupler 440 and distal coupler 440′ is that the through hole 464′ is not positioned between adjacent ribs 460′. Rather, as shown in FIG. 4B, at least one rib 460′ is at least partially interrupted by the through hole 464′.

The second region 472′ of the distal coupler 440′ may have a smooth outer surface because it lacks ribs. For example, instead of ribs the section of the second region 472′ may include overmolded portion, or overmold, formed of a material with a durometer hardness that is similar to the balloon durometer hardness for bonding the balloon to the distal coupler. The material forming the distal coupler is stiffer and/or harder than the material used for the overmolded portion. A laser may be utilized to bond the balloon to the overmolded portion of the distal coupler.

Turning to FIG. 4D, features of a distal coupler 440″ is shown. Like the distal couplers 440,440′ discussed above, the distal coupler 440″ includes a distal face 450″ with an end opening (not visible in this view) defined by a first wall 452″ that extends away from the distal face 450″, a first region 470″ that extends proximally from the distal face 450″ and a second region 472″ that extends proximally from the first region 470″ to the proximal end of the distal coupler 440″ (not visible in this view).

Like the distal couplers 440,440′ shown in FIGS. 4A and 4B, the first region 470″ includes a basket securement assembly comprising a plurality of slots configured to receive the basket splines 442″, a plurality of projections 456″ extending outward from the first wall 452″, and a plurality of second walls 458″ with each second wall 458″ positioned between adjacent projections 456″. Each slot has a distal opening, a proximal opening, and is formed by the first wall 452″, a pair of projections 456″, and a second wall 458″. As discussed above, the projections 456″ may provide protection to the basket during insertion/withdrawal through the introducer. For the distal coupler 440″ illustrated in FIG. 4D, the basket securement assembly further includes an annular wall 466″ that extends from the first wall 452″ and is positioned proximal to the slots. The annular wall 466″ may have a constant height (measured from the outer surface of the first wall 452″ to the outer surface of the annular wall 466″). The proximal side 467″ of the annular wall 466″ may form a stop. The height of the annular wall 466″ may be variable. As shown in FIG. 4D, the distal end 474″ of a basket spline 442″ is positioned in an annular section extending from the distal end of the annular wall 466″ and the proximal end of the projections 456″. The distal end 474″ of the basket spline 242″ is in the form of a barb as discussed in greater detail with reference to FIG. 4F. A benefit of utilizing a barb to secure the basket spline to the coupler is that the interaction between the barb and the basket securement assembly may provide a hard stop to longitudinal movement of the basket spline. For example, as illustrated for the distal coupler 440″, at least a portion of the barb may be aligned with a projection 456″ so that distal movement of the basket spline 442″ is impeded and/or stopped by the barb abutting the at least one projection 456″.

Like the distal couplers 440,440′ shown in FIGS. 4A and 4B, the second region 472″ includes at least one rib 460″ extending from the first wall 452″, a through hole (not visible in this view), and a slot 468″ configured to accommodate a sensor wire. As discussed above, the at least one rib 460″ may provide an increased surface area for attachment of a balloon to the distal coupler 440″. As shown in FIG. 4D, the distal rib 460″ is located adjacent to the annular wall 466″.

The second region 472″ of the distal coupler 440″ may have a smooth outer surface because it lacks ribs. For example, instead of ribs the section of the second region 472″ may include an overmolded portion, or overmold, formed of a material with a durometer hardness similar to the balloon durometer hardness for bonding the balloon to the distal coupler. The material forming the distal coupler may be stiffer and/or harder than the material used for the overmolded portion. A laser may be utilized to bond the balloon to the overmolded portion of the distal coupler.

Turning to FIG. 4E, features of a distal coupler 440′ are shown. Like the distal couplers discussed above, the distal coupler 440′ includes a distal face 450″ with an end opening (not visible in this view) defined by a first wall 452″ that extends away from the distal face 450″, a first region 470″ that extends proximally from the distal face 450″ and a second region 472″ that extends proximally from the first region 470″ to the proximal end of the distal coupler 440′ (not visible in this view).

Like the distal coupler 440″ shown in FIG. 4D, the first region 470″ includes a basket securement assembly comprising a plurality of slots configured to receive the basket splines 442″, a plurality of projections 456″ extending outward from the first wall 452″, a plurality of second walls 458′″ with each second wall 458″ positioned between adjacent projections 456″, and an annular wall 466″. As discussed above, the projections 456″ may provide protection to the basket during insertion/withdrawal through the introducer. Each slot has a distal opening, a proximal opening, and is formed by the first wall 452″, a pair of projections 456″, and a second wall 458″. As illustrated in FIG. 4E, the annular wall 466″ has raised sections 482′ aligned with the projections 456″ and recessed sections 484″ aligned with the slots. Thus, the annular wall 466″ has a variable height. Like the basket securement assembly shown in FIG. 4D, the distal end 474″ of a basket spline 442″ is positioned in the space extending from the distal end of the annular wall 466″ and the proximal end of the projections 456″. As shown, the distal end 474″ of the basket spline 242″ may be in the form of a barb as discussed in greater detail with reference to FIG. 4E. As illustrated in FIG. 4D, the basket spline is inserted into the distal opening of the slot, the distal end of the basket spline exits the proximal opening, and the distal end (barb) expands. A benefit of utilizing a barb to secure the basket spline to the coupler is that the interaction between the barb and the basket securement assembly may provide a hard stop to longitudinal movement of the basket spline. For example, as illustrated for the distal coupler 440′, at least a portion of the barb may be aligned with a projection 456″ so that distal movement of the basket spline 442″ is impeded and/or stopped by the barb abutting the proximal end of a projection 456″.

Like the distal couplers shown in FIGS. 4A-4D, the second region 472″ includes at least one rib 460″ extending from the first wall 452″, a through hole (not visible in this view), and a slot 468′ configured to accommodate a sensor wire. As shown in FIG. 4E, the distal rib 460′ is located adjacent to the annular wall 466″. Between adjacent ribs 460′ the first wall 452″ is flat, like the first wall 452 illustrated in FIG. 4A. As discussed above, the at least one rib 460′ may provide an increased surface area for attachment of a balloon to the distal coupler 440″.

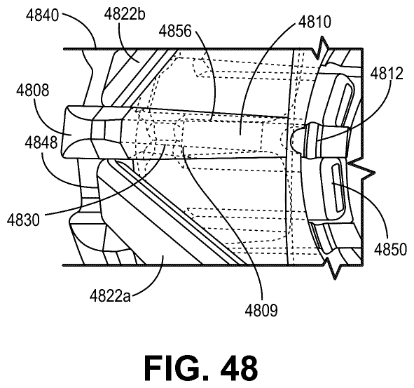

The second region 472′ of the distal coupler 440″ may have a smooth outer surface because it lacks ribs. For example, instead of ribs the section of the second region 472″ may include overmolded portion, or overmold, formed of a material with a durometer hardness similar to the balloon durometer hardness for bonding the balloon to the distal coupler. The material forming the distal coupler may be stiffer and/or harder than the material used for the overmolded portion. A laser may be utilized to bond the balloon to the overmolded portion of the distal coupler. A basket spline may include one or more securement features. The securement features may be configured to secure the basket spline to a coupler (see e.g., FIGS. 4D, 4E, 48, and 58). The spline securement feature may cooperate with a coupler spline retention feature. For example, as discussed above, a spline securement feature may abut a coupler spline retention feature (see e.g., FIGS. 4F-4G and 48). One benefit of a spline securement feature disclosed herein is that axial alignment of the basket splines may be more consistent spline to spline. Additionally, the spline securement feature in cooperation with a coupler spline retention feature may provide a hard distal stop to longitudinal movement of the basket spline. Another benefit of a spline securement feature disclosed herein is that less, to no, adhesive may be required to secure the spline to the coupler because the spline securement feature and the coupler securement feature cooperate to anchor the basket spline. The spline retention feature may be positioned at one or both ends of the basket spline (see e.g., FIGS. 4F-4G) or positioned between a bend in the spline and an end of the spline (see e.g., FIG. 48). A spline retention feature may extend from the side of the basket spline (see e.g., FIG. 48, hereinafter referred to as a side extending spline retention feature). Two side extending spline retention features may be positioned opposite one another. A side extending spline retention feature may be a wider section of the spline.

Turning to FIGS. 4F-4G a spline retention feature is positioned at the end of the basket spline are illustrated. The spline retention feature may include a barb. The barb may secure the basket spline to a coupler, as illustrated in FIGS. 4D and 4E. One benefit of utilizing a barb to secure the splines of a basket to a coupler is that the axial alignment may be more consistent spline to spline. This may improve basket performance. Additionally, as discussed above, the barb may provide a hard distal stop to longitudinal movement of the basket spline. Another benefit may be that less, to no, adhesive may be required to secure the spline to the coupler. Thus, the barb may anchor the basket spline to the distal coupler.

Turning to FIG. 4F, a barb 478 may include two projections 483a, 483b extending from a base 481. The width of the base 481 may be smaller than the width of the basket spline 442. In this embodiment, the base 481 is centered—i.e., positioned an equal distance from the sides of the basket spline 442a. Each projection 483 includes a head 485 (head 485b identified) configured to provide a 180° turn to the projection 483. The 180° turn positions the free end 487 (free end 487b identified) of the projection 483 adjacent to the base 481. Thus, the projection 483 may be described as V-shaped or U-shaped. A first slot 488 is defined between the projection 483 and the base 481. A second slot 490 is defined by the two projections 483a, 483b. The second slot 490 may include a keyhole 489 (keyhole 489a is identified). The barb 478 may transition between a contracted state and an expanded state. The barb 478 illustrated in FIG. 4F is in an expanded state. For example, when the basket spline is inserted into the slot of a coupler the barb may be in a contracted state and when the basket spline exits the slot opening the barb may transition to an expanded state (see e.g., FIG. 4D). In the contracted state the first slot 488 may have a first size because the projections 483 are positioned a first distance from the base 481 and in the expanded state the first slot 488 has a second size greater than the first size because the projections 483 are positioned a second distance, greater than the first distance, from the base 481.

Turning to FIG. 4G, a barb 479 may include a first projection 492 and a second projection 494 each extending from a base 491. The width of the base 491 may be smaller than the width of the basket spline 442b. The base 491 may be offset—i.e., the base 491 is positioned closer to one side of the spline 442b than to the other side of the spline 442b. The first projection 492 includes a free end 493. The second projection 494 includes a head 495 positioned adjacent to the free end 493 of the first projection 492 and a free end 496 positioned adjacent to the base 491. Like the head 485 of barb 478 shown in FIG. 4F, the head 495 is configured to provide a 180° turn to the projection 494. The 180° turn positions the free end 496 of the projection 494 adjacent to the base 491. Thus, the projection 494 may be described as V-shaped or U-shaped. As shown, the head 495 illustrated in FIG. 4G has a smaller longitudinal length than head 485 illustrated in FIG. 4F. A first slot 497 is defined by the first projection 492 and the second projection 494. A second slot 498 is defined by the second projection 494 and the base 491. The second slot 498 includes a keyhole 499. The barb 479 may transition between a contracted state and an expanded state. The barb 479 illustrated in FIG. 4G is in an expanded state. For example, when the basket spline is inserted into the slot of a coupler the barb may be in a contracted state and when the basket spline exits the slot opening the barb may transition to an expanded state (see e.g., FIG. 4E). In the contracted state the second slot 498 may have a first size because the projection 494 is positioned a first distance from the base 491 and in the expanded state the second slot 498 has a second size greater than the first size because the projection 494 is positioned a second distance, greater than the first distance, from the base 491.

The distal coupler 440 may be configured to be secured to a shaft of a catheter. For example, the shaft may be inserted into a central lumen (such as central lumen 604 shown in FIG. 6) of the distal coupler 440. The distal coupler 440 may be attached to the shaft by a friction fit. The first wall 452 may define at least one mating feature, such as a through hole, that is configured to secure the distal coupler 440 to the shaft of a catheter. The mating feature may be a through hole. Adhesive may be deposited into the through hole. The through hole may be utilized to weld the distal coupler 440 to the shaft. A through hole may be a circular opening with a diameter (such as through hole 464 with diameter D4 in FIG. 6) or a slit, a longitudinal opening with a width, (such as slot 468 with width W1 in FIG. 5). The mating feature may be positioned in the second region 472 of the distal coupler 440. For example, as illustrated in FIG. 4A, a single through hole 464 may be positioned between two ribs 460. A through hole, such as through hole 564 illustrated in FIG. 5, may additionally or alternatively be positioned adjacent to an end of the distal coupler 440.

A spline of a basket configured to be secured to a distal coupler illustrated in FIGS. 4A-4E, may include a bend, such as bend 448, so that the end of the spline 442 may be inserted into a slot of the basket securement assembly. For example, as illustrated in FIG. 4A, the spline section 446 that extends from a spline section 444 to an end of the spline 442 includes the bend 448. At least one of spline section 446 and spline section 444 may have an insulative cover/coating. The spline 442 may be pliable. For example, the bend 448 may straighten as the spline advances beyond the distal face of the coupler and a temporary secondary bend may form at a different location of the spline.

Turning to side view of the distal coupler 440 illustrated in FIG. 5, the projections 456 may include a curved leading section 508 and a curved trailing section 510. The curved leading section 508 may extend upwards and proximally from the distal face of the distal coupler 440. The curved leading section 508 has a radius of curvature R1. The exterior surface/side 506 of the projection may extend at an angle from the curved leading section 508 to the curved trailing section 510. The curved trailing section 510 has a radius of curvature R2. The radius of curvature R2 for the trailing section 510 may be smaller than the radius of curvature R1 for the leading section 508 (R1>R2). For example, in one embodiment, radius of curvature R1 is about three times larger than radius of curvature R2. The curved trailing section 510 may terminate at the wall 412 (best shown in FIG. 6).

As illustrated in FIG. 5, the distal coupler 440 has a length measured from the distal face 450 to the proximal end 502, hereinafter referred to as a coupler length, that is equal to the sum of lengths L1, L2, L3, L4, L5, L6, and L7. The first region 470 has a length equal to the sum of length L1 and length L2 (L1+L2) and the second region 472 has a length equal to the sum of L3+L4+L5+L6+L7. The length of the first region 470 may be less than the length of the second region 472. For example, the length of the first region 470 may be about 32-42% of the coupler length and the length of the second region 472 may be about 57-67% of the coupler length. Length L1 represents the distance measured from the distal face to the center of the through hole 462 with diameter D1. Length L2 represents the distance measured from the center of the through hole 462 to the end of the first region 470 of the coupler. The length L2 may be about 13-23% of the length of the first region 470. As shown, the distal coupler 440 has three ribs. Length L3 represents the distance measured from the end of the first region 470 to a first rib, length L4 represents a distance measured between adjacent ribs with a through hole, such as through hole 464, positioned therebetween, and length L5 represents a distance measured between adjacent ribs with no through hole positioned therebetween, where length L3 is measured from the proximal end of the first region 470 to a midpoint of the distal rib and length L4 and length L5 are measured from the midpoints of longitudinally adjacent ribs. Length L3, length L4, and length L5 may be the same or different. The length between adjacent ribs with a through hole positioned therebetween may be larger than the lengths between adjacent ribs with no through hole positioned therebetween. As illustrated in FIG. 5, L3<L5<L3. Length L6 is the distance between a midpoint of a rib and an outlet for wires, such as sensor wires 386 shown in FIG. 3B, to exit the lumen of the distal coupler. Length L7 is the distance measured between the proximal end 502 of the coupler and the outlet for wires, such as sensor wires 386 shown in FIG. 3B, to exit the lumen of the distal coupler 440. In some embodiments, length L7 is greater than length L1. The sum of length L6 and length L7 is a distance measured between the proximal end 502 of the distal coupler 440 and a proximal rib. Diameter D2 represents the diameter of a rib 460 located in the second region 472.

Turning to FIG. 6, a cross-sectional view of FIG. 5 taken along line A-A, the distal coupler 440, with longitudinal axis 606, is a one piece or unitary design with the first wall 452 extending from the distal face 450 to proximal end 502 and defining a central lumen 604. A coupler with a unitary design may be easier to manufacture, more cost effective to manufacture and/or has improved performance compared to a coupler comprising multiple components. For example, in at least one embodiment, the distal coupler 440 is manufactured by molding. The distal end of a shaft, such as the support shaft 382 shown in FIG. 3B, may be positioned in the central lumen 604 and bonded to the distal coupler.

The first wall 452 may have a variable thickness. For example, in the first region 470, a section of the first wall 452 at the distal face 450, with length L8, may be thicker to define the end opening 454. The remaining section of the first wall 452 in the first region 470 may be thinner. The remaining portion of the first wall 452 in the first region 470 may be the thinnest section of the first wall 452. As illustrated in FIG. 6, the first wall 452 adjacent to the first region 470 may be thicker than the rest of the first wall 452. A section 608 of the first wall 452, with length L11, may be thinner than the rest of the first wall 452 in the second region 472.

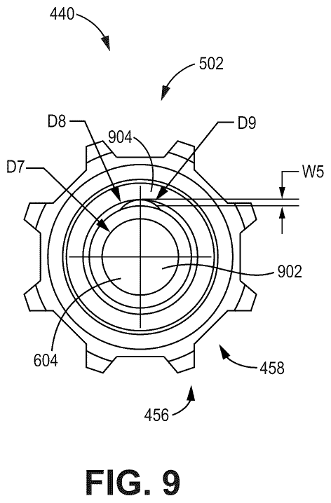

The central lumen 604 may have a uniform diameter. The central lumen 604 has a first diameter and a second diameter less than the first diameter. For example, as illustrated in FIG. 6, the section of the central lumen extending from the proximal end 502 to edge 610 has a larger diameter (such as diameter D8 shown in FIG. 9) than the section of the central lumen extending from edge 610 to edge 614. The larger diameter section of the central lumen 604 may have a length L10 that overlaps at least partially with lengths L7, L6, and/or L5 shown in FIG. 5. The length L10 may be about 34%-44% of the coupler length.

As discussed above, the central lumen 604 may be configured to receive a shaft of a catheter. A shaft positioned within the central lumen may abut the edge 610 of the first wall 452. A shaft positioned within the central lumen may abut edge 614.

The first wall 452 may include a sensor alignment feature configured to align a sensor positioned in the central lumen 604. The sensor alignment feature may also limit and/or prevent forward movement of the sensor. The edge 610 may be the distal end of the sensor alignment feature and prevent forward movement of a sensor positioned within the central lumen. The edge 614 may be the distal end of the sensor alignment feature and prevent forward movement of a sensor positioned within the central lumen. The sensor may extend beyond the first region 470 by a distance L9.

Wall 412 is perpendicular to the first wall 452. Second wall 458, extending parallel to the first wall 452, is perpendicular to wall 412. As illustrated, the second wall 458 is connected to wall 412. Second wall 458 may not be connected to the wall 412. For example, as shown in FIG. 4B, the proximal end of second wall 458 is distal to the wall 412. The distal coupler may lack the wall 412, as shown in FIGS. 4D and 4E.

Additional measurements identified in FIG. 6 include diameter D3, length L9, and width W2. Diameter D3 is the outer diameter of the first wall 452 in the second region 472 of the coupler. Diameter D3 is less than diameter D2 shown in FIG. 5 (D2>D3). Length L9 represents the distance measured from wall 412 of the distal coupler 440 to the center of the through hole 464 and overlaps at least partially with lengths L3 and/or L4 shown in FIG. 5. The sum of lengths L2+L9 represents the clearance/distance between mating features, such as through hole 462 and through hole 464. As illustrated in FIGS. 5 and 6, length L9 is less than the combination of lengths L3 and L4 (L9<(L3+L4)). The slot 602 has a width W2 measured between the outer surface of the first wall 452 and the inner surface of the second wall 458. As discussed above, there is a slot 504 distal to the slot 602 configured to guide a spline moving beyond the distal face of the coupler.

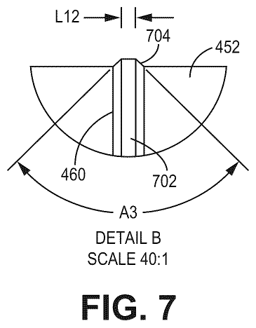

Turning to FIG. 7, features of the rib 460 are illustrated. The rib 460 has a center section 702 positioned above the outer surface of the first wall 452 and sides 704 extending at an angle from the center section 702 to the first wall 452. As illustrated in FIG. 7, the center section 702 has a longitudinal width L12 and the angled sides 704 of a rib 460 are oriented at an angle A3. The angle A3 may be 90° or an oblique angle. As discussed above, the ribs 460 may be a reinforcement feature configured to provide mechanical strength to the coupler.

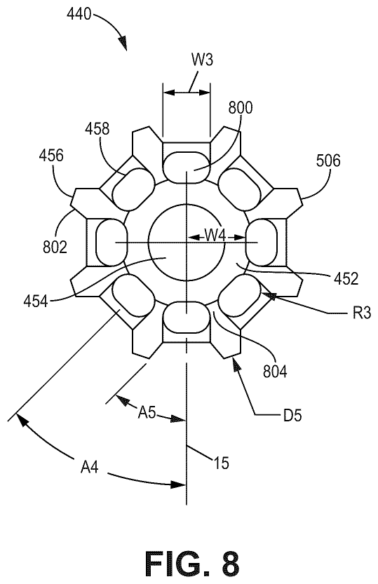

The distal face of a coupler disclosed herein may be symmetrical (best shown e.g., in FIGS. 4A, 8, and 51). For example, as illustrated in the end view provided in FIG. 8 of the distal coupler shown in FIG. 4A, the distal face 450 is radially symmetrical with a plurality of openings 800 of slots configured to receive a basket spline, such as slot 602 shown in FIG. 6 (hereinafter referred to as slot openings 800). Each slot opening 800 is positioned opposite another slot opening 800 (see axis 14), each second wall 458 is positioned opposite another second wall 458, and each projection 456 is positioned opposite another projection 456. Likewise, each slot is positioned opposite another slot. The slot openings 800 may be rounded with a radius of curvature R3. Each projection 456 may include an exterior surface 506 that is wider than an inner surface 804. Each projection 456 may further include side surfaces 802 oriented at an oblique angle A5 measured from a line 15 that bisects the slot and is normal to the surface of the second wall.

As illustrated, the distal coupler 440 has eight (8) slot openings 800 with adjacent slot openings 800 oriented at an oblique angle A4 measured between the midpoints of adjacent slot openings 800. Width W3 represents the width of the second walls 458 as well as a distance between adjacent projections 456. Width W4 represents the distance from the center of the end opening 454 to an outer surface of the section of the first wall 452 forming the slot 504. Diameter D5 represents the diameter measured between the exterior sides of oppositely positioned projections 456.

A view of the distal coupler 440, looking from the proximal end 502 towards the distal face 450, is provided in FIG. 9. The proximal end may be symmetrical. For example, the proximal end may be bilaterally symmetrical about axis 902. Diameter D7 represents the diameter of the end opening 454. The diameter D7 may be configured to act as a hard stop to a magnetic sensor positioned in the central lumen 604 of the distal coupler 440. In other words, the diameter D7 is smaller than the diameter of the magnetic sensor. Slot 904 has a thickness W5 and a diameter D9. The thickness W5 and the diameter D9 may be configured to provide clearance to magnetic sensor termination to the sensor wires. The diameter D8 is sized to mate with a shaft, such as the support shaft 383 shown in FIG. 3C.

Turning to FIG. 48, features of a basket securement assembly/region of a distal coupler 4840 are illustrated. Like the distal coupler 440 discussed above, the basket securement assembly of the distal coupler 4840 may include a longitudinally extending projection 4856. The projection 4856 may extend parallel to the longitudinal axis of the distal coupler 4840. In contrast to projections 456, 456′, 456″, 456′″ discussed above, projection 4856 is discontinuous and defines a retention slot 4809 between a curved leading section 4808 and a trailing section 4810. The trailing section 4810 may include a projection or rib 4812 extending across the trailing section 4810 from one side to the other. As illustrated in FIG. 48, the retention slot 4809 is configured to receive a side extending spline retention feature 4830 extending from one or two adjacent splines, collectively 4822. The spline retention feature 4830 may snap fit into the retention slot 4809. Although retention slot 4809 is configured to receive a side extending spline retention feature 4830, the distal coupler 4840 may also be utilized to secure basket splines lacking a spline retention feature. The splines 4822 illustrated in FIG. 48 include two side extending retention features 4830 positioned at the same longitudinal location along the spline 4822 and extending from opposite sides of the spline 4822. Further, the illustrated spline retention features 4830 are positioned adjacent to a bend 4848 in the spline 4822, a distance away from the end 4850 of the spline 4822. However, the spline retention features 4830 may be located at any position between the bend 4848 and the end 4850 of the spline 4822.

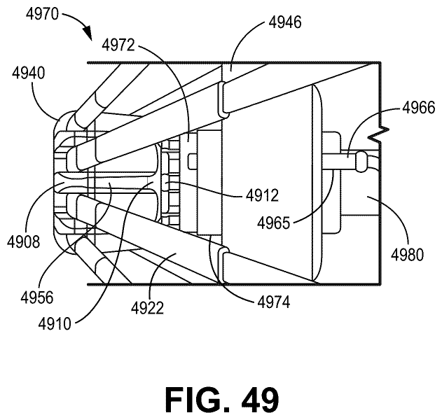

FIGS. 49 and 50 are isometric and cross-sectional views illustrating features of a distal coupler 4940. The distal coupler 4940 includes a first region 4970 and a second region 4972 extending proximally from the first region 4970. As illustrated in FIG. 50, the first and second regions 4970, 4972 may be a unitary structure. A shaft 4980 may extend into a proximal end opening of the distal coupler 4940 so that a portion of the shaft 4980 is positioned in the central lumen 5004 of the distal coupler 4940. The distal end of the shaft 4980 may abut a stop 5074 (best shown in FIG. 50). The stop 5074 may be located in the second region 4972 of the distal coupler 4940.

The first region 4970 may include a basket securement assembly configured to couple a plurality of splines 4922. The features of the basket securement assembly are similar to the basket securement assembly provided in the first region 470 shown in FIG. 5. In contrast to the first region 470 of distal coupler 440, the first region 4970 lacks through holes 462. The basket securement assembly provided in the first region 4970 includes plurality of longitudinally extending projections 4956 and a plurality of circumferentially extending projections 4912. Like the projections 456, 456′, 456″, 456″ discussed above, the projections 4956 are continuous. Each longitudinally extending projection 4956 may include a curved leading section 4908 and a trailing section 4910. The trailing section 4910 may be curved as discussed above.

The second region 4972 of the coupler may include a sensor and/or a balloon securement region. For example, the distal coupler 4940 may include a slot 4965 configured to receive a sensor 4966. Sensor 4966 may be utilized for navigation, e.g., a magnetic or electromagnetic sensor discussed above. Slot 4965 is defined in an outer surface of the second region 4972 of the distal coupler 4940. Since the longitudinal axis of the slot 4965 is offset and parallel to the longitudinal axis of the catheter, a sensor 4966 positioned in the slot 4965 will be offset and parallel to the longitudinal axis of the catheter. In this location, the sensor 4966 will not come into contact with any biological fluids that may enter the lumen 5004. As shown in FIG. 50, the sensor 4966 may be proximally oriented, i.e., the sensor wires 5067 extend proximally from the proximal end of the sensor 4966, as discussed above.

As illustrated in FIGS. 49 and 50, a portion of the balloon 4946, e.g., a distal waist, may be coupled to the second region 4972 of the distal coupler 4940 so that the balloon 4946 extends over the slot 4965 and over a sensor 4966 positioned in the slot 4965. This arrangement, securing the balloon 4946 over the sensor 4966, may improve reach and/or the ability to target desired anatomic locations with the catheter. The second region 4972 may include an overmolded thermal plastic region 4974 configured for thermal welding of the balloon 4946 to the distal coupler 4940.

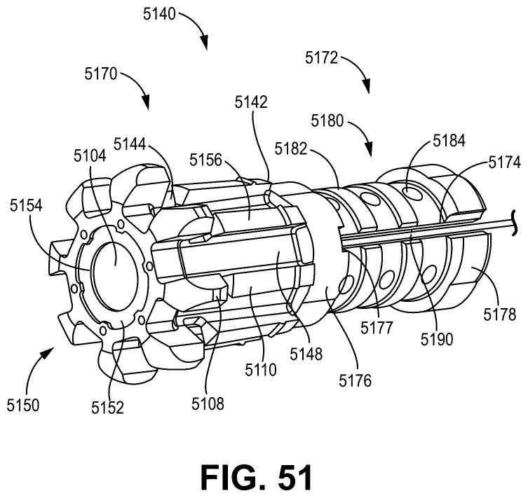

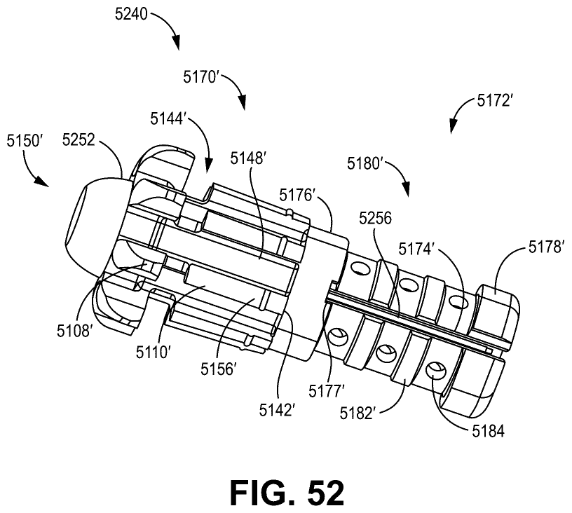

A distal coupler as discussed above may include at least one electrode positioned distal to an expandable end assembly. The electrode may be either in addition to a magnetic sensor or instead of a magnetic sensor. For example, an electrode coupled to the distal coupler may be utilized for tracking, e.g., impedance tracking, of the distal end of the expandable end assembly. Shaft electrodes positioned proximal to the expandable end assembly may also be utilized for impedance tracking. Utilizing an electrode for tracking may reduce the cost of the medical device and/or the cost of utilizing the medical device. For example, an electrode may cost less than a magnetic sensor. As another example, a magnetically generated field and/or hardware would not be required to determine the location and/or orientation of an end assembly coupled to the distal coupler when using the medical device during a procedure. Thus, cost to the user may be reduced. The electrode may be coupled to the distal face of the distal coupler (hereinafter referred to as a tip electrode). The shape of the tip electrode may be atraumatic. The tip electrode may be flush with the distal face of the distal coupler (see e.g., FIG. 51) or may extend distally from the distal face of the distal coupler (see e.g., FIG. 52). An electrode extending distally from the distal face may be described as a bulbous electrode. In one example, the distal face of the distal coupler may define a recess configured to receive the tip electrode. The outer diameter of the tip electrode may be less than the outer diameter of the distal face. The tip electrode may define a lumen in communication with the central lumen of the distal coupler. A tip electrode lumen may provide a passageway for a guidewire to exit the distal coupler and/or for fluid, e.g., contrast. An electrode coupled to the distal coupler may further be utilized for ablation and/or mapping.

Turning to FIG. 51, features of a distal coupler 5140 are illustrated. Like the distal couplers discussed above, distal coupler 5140 includes a second region 5172 extending proximally from a first region 5170. The first and second regions 5170, 5172 may be a unitary construction. Alternatively, one of the first or second regions 5170, 5172 may be a female section while the other of the first or second regions 5170, 5172 may be a male section. For example, the second region 5172 may include a male connector received by the first region 5170. The connection may further include a poka yoke alignment/retention feature to orient male/female connectors.

The first region 5170 may include a basket securement assembly comprising a plurality of slots or grooves 5148 and a plurality of projections 5156. Projections 5156 are discontinuous and include distal projections 5108 and proximal projections 5110 defining retention slots 5144 configured to receive spline retention features of basket splines, as discussed above. The outer diameter of the proximal projections 5110 may be greater than the outer diameter of the annular wall 5176. Thus, the proximal end 5142 may function as a stop. As illustrated in FIG. 51, the distal coupler 5140 is configured to secure a basket with eight splines. However, as discussed above, the number of slots and projections may be modified to secure a basket with more or fewer than eight splines. An annular wall 5176 may be positioned at the proximal end of the first region 5170. The annular wall 5176 may function as a stop limiting proximal movement basket splines positioned in the slots 5148. The outer diameter of the annular wall 5176 may be less than the outer diameter of the proximal projections 5156.

The second region 5172 includes a distal section 5180 and a proximal annular wall 5178. The proximal annular wall 5178 may form the proximal end of the second region 5172. The second region 5172 defines a slot 5174 configured to route an electrical lead 5190 coupled to the electrode 5152. The slot 5174 extends distally from the proximal end of the distal coupler 5140. As illustrated in FIG. 51, the slot 5174 extends through the proximal annular wall 5178, the distal section 5180, and forms a notch 5177 in the annular wall 5176. The second region 5172 may include one or more annular ribs 5182 and/or one or more through holes 5184. The distal coupler 5140 illustrated in FIG. 51 has two annular ribs 5182 (a distal annular rib and a proximal annular rib) and a plurality of through holes 5184 arranged in rows with a first through hole positioned between the annular wall 5176 and a distal annular rib, a second through hole positioned between the two annular ribs, and a third through hole positioned between a proximal annular rib and the proximal annular wall 5178. As discussed above, the one or more annular ribs 5182 may be utilized to attach a balloon to the distal coupler 5140 and the through holes 5184 may be utilized to attach the distal coupler 5140 to a shaft positioned in the central lumen 5104 of the distal coupler 5140.

In this example, the distal coupler 5140 further includes an electrode 5152. As illustrated in FIG. 51, the electrode 5152 is a ring positioned in a recess in the distal face 5150 of the distal coupler 5140. This arrangement may provide the medical device with a flat distal face which may improve contact of the distal face with a target site during use. Lands and grooves may be utilized to couple the electrode 5152 to the distal face of the first region 5170 of the distal coupler 5140. For example, the electrode 5152 may include at least one groove and the recess in the distal face 5150 may include at least one land. The electrode 5152 may be pressed fitted or swaged into the recess. The electrode 5152 defines a lumen 5154. The lumen 5154 of the electrode 5152 may be coextensive with the central lumen 5104 of the distal coupler 5140, e.g., the inner diameters of the lumens 5104 and 5154 are the same. The lumen 5154 may be sized for a guidewire and/or to deliver fluid, e.g., contrast.

FIG. 52 illustrates a distal coupler 5240 with an electrode 5252 extending distally from the distal face of the distal coupler 5240 but includes many of the same features of the distal coupler 5140 shown in FIG. 51 and discussed above. For example, the distal coupler 5240 includes a distal face 5150′, a first region 5170′ that includes a basket securement assembly comprising a plurality of slots or grooves 5148′ and a plurality of projections 5156′ that are discontinuous and include distal projections 5108′ and proximal projections 5110′ defining retention slots 5144′ configured to receive spline retention features of basket splines and with a proximal end 5142′ that may function as a stop, an annular wall 5176′ defining a notch 5177′, a second region 5172′ that includes a distal section 5180′, a proximal annular wall 5178′, one or more annular ribs 5182′, one or more through holes 5184′, and defines a slot 5174′, configured to route an electrical lead 5256 coupled to the electrode 5252, that extends through the proximal annular wall 5178, the distal section 5180, and forms a notch 5177 in the annular wall 5176. As shown, the electrode 5252 forms the distal face 5150′.

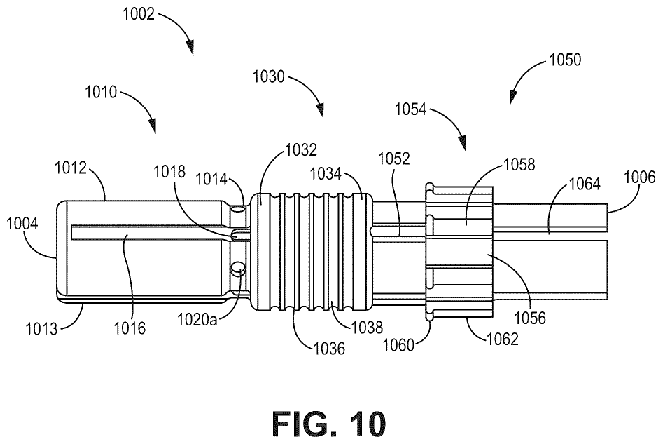

As discussed above, the catheter may include a proximal coupler. Features that may be included in a proximal coupler are illustrated in FIGS. 10-20B and 53-63. Turning to FIG. 10, an isometric view of a proximal coupler 1002 configured to secure a basket to a catheter is provided. The proximal coupler 1002 may be a single piece construction. For example, the proximal coupler 1002 may be molded. The proximal coupler 1002 includes a distal end 1004, a proximal end 1006, a distal region 1010, a first securement region 1030 extending proximally from the distal region 1010, and a proximal region 1050 extending proximally from the first securement region 1030.

The distal region 1010 may include a larger diameter region 1012, a smaller diameter region 1014, at least one groove configured to receive a sensor, and at least one through hole. In the view provided in FIG. 10, a sensor groove 1016 is oriented parallel to the longitudinal axis of the proximal couple 1002. As shown, the sensor groove 1016 may be positioned along the outer surface of the larger diameter region 1012. The sensor groove 1016 may be sized to receive a magnetic sensor. The smaller diameter region 1014 includes at least one through hole. The at least one through hole may include through holes, such as through hole 1018, for one or more sensor wires to extend from the exterior of the proximal coupler 1002 into the lumen of the proximal coupler 1002 and/or at least one hole, such as hole 1020a (collectively 1020), configured to supply a fluid out from the lumen of the proximal coupler 1002 (hereinafter media holes). As shown, the proximal coupler 1002 has a plurality of media holes 1020.

The first securement region 1030 may be configured for securing a proximal end of an inflatable balloon to the proximal coupler 1002. The first securement region 1030 includes a plurality of ribs that include a distal side rib 1032, at least one middle rib 1036, and a proximal side rib 1034. Like the ribs of the distal coupler embodiments discussed above, the ribs of the proximal coupler provide an increased surface area for attaching a balloon. As shown in FIG. 10, the surfaces 1038 between adjacent ribs are curved. As discussed above this increases the surface area compared to a flat surface. However, the surfaces between adjacent ribs may be flat. The first securement region 1030 may lack ribs and have an overmolded portion, an overmold, as discussed above for the distal coupler embodiments. Inflation media may be supplied via the media holes 1020 to a balloon coupled to the first securement region 1030.

The proximal region 1050 includes a second securement component 1054 configured to secure basket splines to the proximal coupler 1002, and at least one slot configured to secure the proximal coupler 1002 to a catheter shaft. As illustrated, the second securement component 1054 may include a plurality of projections/lands 1058 and a plurality of grooves 1056. A wall 1060 may extend outward from the body 1062 of the land 1058. As illustrated, the wall 1060 is at the distal end of the land 1058. The proximal region 1050 may further includes at least one longitudinal ridge for routing one or more wires, and at least one slot configured to secure the proximal coupler 1002 to a catheter shaft. The at least one longitudinal ridge includes a first ridge 1052. As illustrated, the first ridge 1052 is oriented parallel to the longitudinal axis of the proximal coupler 1002. The at least one slot includes a first slot 1064 extending through the wall of the proximal coupler 1002. As illustrated, the first slot 1064 is oriented parallel to the longitudinal axis of the proximal coupler 1002.

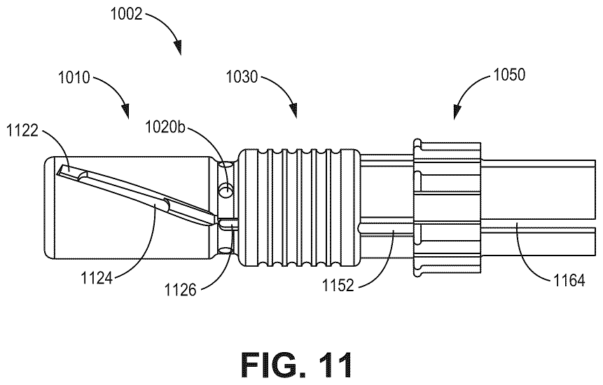

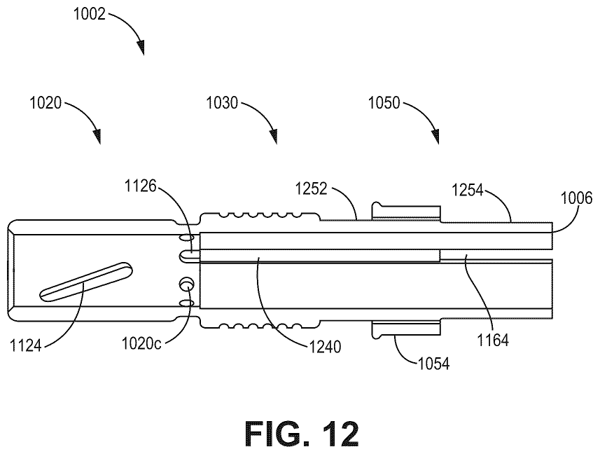

An isometric view of the proximal coupler 1002 rotated 180 from the view of FIG. 10 is provided in FIG. 11. As shown in FIG. 11, the proximal coupler further includes a second groove, groove 1122, configured to receive a sensor. The sensor groove 1122 is positioned 180° from sensor groove 1016 and is oriented at a 20° angle to the longitudinal axis of the proximal coupler 1002. Orienting the magnetic sensor at a 20° angle may improve accuracy of the magnetic sensor. In contrast to groove 1016, the diagonal groove 1122 includes an opening 1124 positioned between the ends of the diagonal groove 1122. Sensor wires extending proximally from a sensor positioned in groove 1122 may pass through hole 1126. As shown in FIG. 11, the proximal region 1050 may include a second longitudinal ridge 1152 and a second longitudinal slot 1164. The second longitudinal ridge 1152 corresponds to a groove, such as groove 1240 shown in FIG. 12.

A longitudinal cross-sectional view of FIG. 10 is provided in FIG. 12. A groove 1240 in the inner surface of the coupler wall extends proximally from the hole 1126 to the second longitudinal slot 1164. Groove 1240 may be utilized for routing sensor wires through the proximal coupler 1002. Two additional grooves 1240 in the inner surface of the coupler wall may be utilized to route sensor wires for a distal sensor and for the other sensor coupled to the proximal coupler 1002. The proximal region 1050 has a variable diameter. For example, the section 1254 extending from the proximal end 1006 to the proximal end of the second securement component 1054 has a diameter that is less than the diameter of the section 1252 extending from the proximal end of the second securement component 1054 to the proximal end of the first securement region 1030. As discussed below in greater detail, the smaller diameter section may be inserted into a catheter shaft.

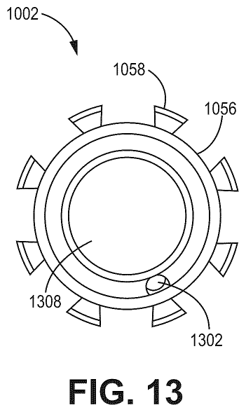

FIG. 13 is an end view of the proximal coupler 1002 showing the lumen 1308, a second securement region with lands 1058 and grooves 1056, and the opening 1302 for a groove, such as groove 1013, for routing one or more wires extending from a sensor positioned in a distal coupler.

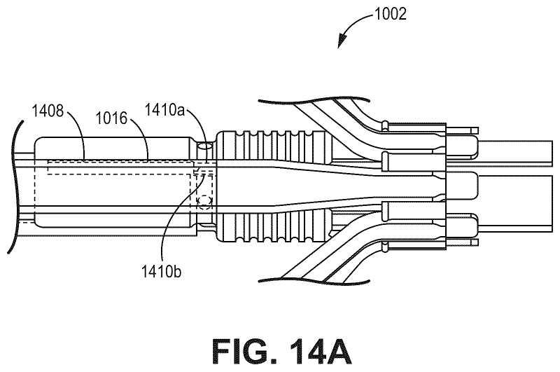

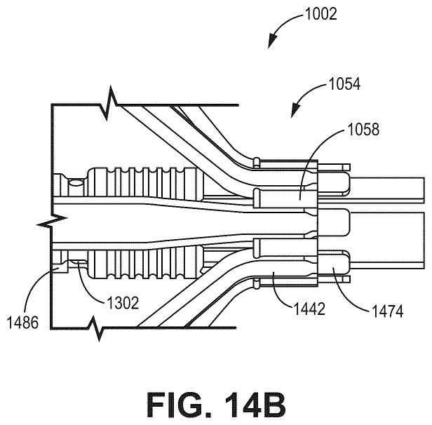

FIGS. 14A and 14B are isometric views showing basket splines 1442 coupled to the second securement component 1054 and a sensor wire 1486 positioned in the groove 1013. In the view provided in FIG. 14A the basket splines 1442 extending over the proximal coupler 1002 are transparent. The view also shows a magnetic sensor 1408 positioned in groove 1016 with two sensor wires 1410a, 1410b extending proximally from the magnetic sensor 1408. The magnetic sensor 1408 may be potted into the groove 1016. When a balloon is coupled to the proximal coupler 1002, the magnetic sensor 1408 will be positioned within the balloon lumen and exposed to the inflation media. The magnetic sensor 1408 may be covered by a layer of material.

As shown in FIG. 14B, to secure the basket splines 1442 to the second securement component 1054, the basket splines 1442 are positioned in the grooves 1013 between adjacent lands 1058. The proximal ends 1474 of the basket splines 1442 are positioned proximal to the proximal end of the second securement component 1054. A space separates the proximal ends 1474 from the outer surface of the proximal coupler 1002.

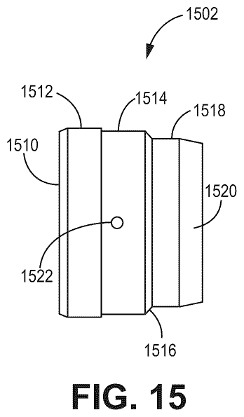

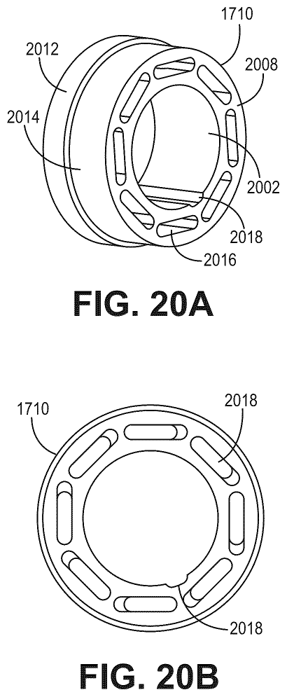

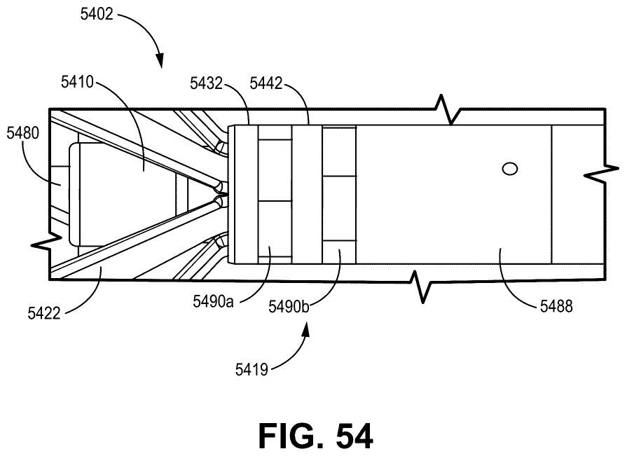

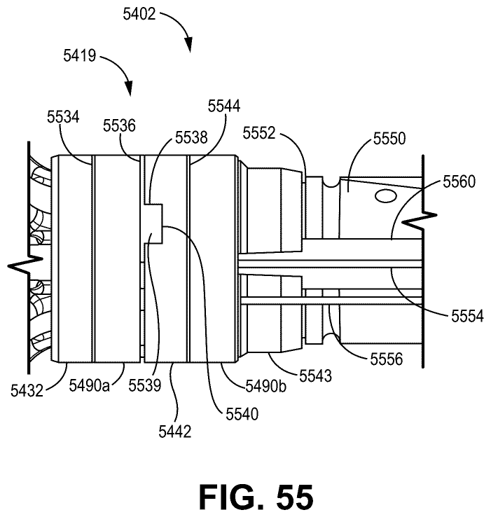

The catheter may include a retention ring (see e.g., FIGS. 15, 20, 52-55). The retention ring may be a unitary retention ring (see e.g., FIGS. 15 and 20) or multi-component retention ring (see e.g., FIG. 54-55). The retention ring may secure the basket splines to a coupler. One or more ring electrodes may be positioned around and/or secured to the retention ring. Positioning one or more ring electrodes around the retention ring may provide several benefits. For example, positioning a ring electrode around the retention ring may reduce the distance between the ring electrode and the proximal end of the basket. As another example, positioning a plurality of ring electrodes around the retention ring may reduce the intra ring electrode spacing (i.e., distance between adjacent ring electrodes). As one non-limiting example, the intra ring electrode spacing may be reduced by 67% to 77%. Yet another benefit is that the length between the basket and the deflecting portion of the deflectable shaft (deflectable pocket length) may be reduced. For example, the length between the basket and the deflecting portion of the deflectable shaft may be reduced by 35% to 43%. As another example, utilizing a retention ring as disclosed herein may reduce the length from the distal end of the shaft to the deflectable portion of the shaft. For example, the length from the distal end of the shaft to the deflectable portion of the shaft may be reduced by 66% to 76%. Reducing one or more of these lengths may reduce the deflectable pocket length of the catheter. Reducing the deflectable pocket length may improve vein accessibility.

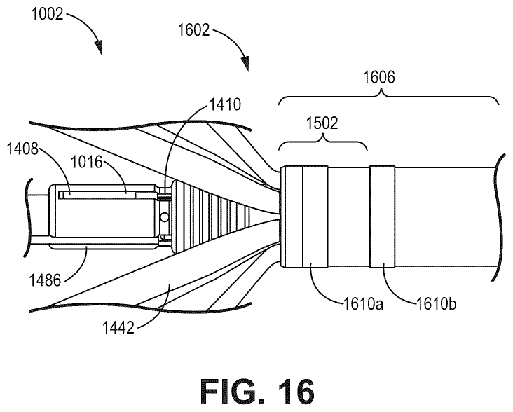

Turning to FIG. 15, an isometric view of a unitary retention ring 1502 configured to be positioned around the proximal coupler 1002 and/or basket splines (such as basket splines 1442 shown in FIG. 16), is provided. The retention ring 1502 may include a plurality of annular sections defining a lumen sized to surround a portion of the proximal coupler 1002. As illustrated, the retention ring 1502 includes a first annular section 1510 forming a first end of the retention ring 1502, a second annular section 1512 extending from the first annular section 1510, a third annular section 1514 extending from the second annular section 1512, a fourth annular section 1516 extending from the third annular section 1514, a fifth annular section 1518 extending from the fourth annular section 1516, and a sixth annular section 1520 extending from the fifth annular section 1518 and forming a second end of the retention ring 1502. The first annular section 1510 may form the distal end of the retention ring 1502 and the sixth annular section 1520 forms the proximal end of the retention ring 1502. The outer diameter and/or inner diameter of the retention ring 1502 may be variable. For example, in this embodiment, the outer diameter of the second annular section 1512 (od1) is greater than the outer diameter of the third annular section 1514 (od3) and the outer diameter of the fifth annular section 1518 (od5) is less than the outer diameter of the third annular section 1514 (od3). The retention ring 1502 may also have a variable inner diameter. For example, as discussed below in greater detail, the second annular section 1512 may have a first inner diameter along the distal end and a second inner diameter less than the first inner diameter. Additionally, some of the annular sections may be angled. For example, in this embodiment, the first annular section 1510, the fourth annular section 1516, and the sixth annular section 1520 are oriented at an angle to the longitudinal axis of the retention ring 1502. As illustrated, the first annular section 1510 extends downward from the second annular section 1512, the fourth annular section extends downward from the third annular section 1514, and the sixth annular section 1520 extends downward from the fifth annular section 1518. The angled surface of the first annular section 1510 may abut the distal side of the walls 1060 of the lands 1058. This arrangement may function as a hard stop for the retention ring 1502 to limit proximal movement of the retention ring 1502 (as shown in FIG. 16). The retention ring may further include at least one hole 1522. The hole 1522 may be a blind hole or a through hole for securing a shaft electrode to the retention ring 1502.

FIG. 16 is an isometric view showing the retention ring 1502 positioned around the basket splines 1442 coupled to the second securement component 1054 and a shaft 1606 with one or more electrodes 1610 is positioned over the retention ring 1502. The proximal coupling 1602 may be a multi-component comprising the proximal coupler 1002 and the retention ring 1502.

As shown in FIG. 16, the retention ring 1502 may include one or more features that act as a hard to stop to limit distal and/or proximal movement of the retention ring 1502. For example, the distal end of the retention ring 1502 may be distal to the walls 1060 of the lands 1058. The smaller second inner diameter of the second annular section 1512 may form a surface that abuts the proximal side of the walls 1060 of the lands 1058. This arrangement may function as a hard stop for the retention ring 1502 to limit distal movement of the retention ring 1502. Similarly, the inner diameter of the first annular section 1510 may extend downward so that the first annular section 1510 is positioned adjacent to the distal surface of the walls 1060 of the lands 1058. The proximal end of the third annular section 1514 may be aligned with the proximal end of the second securement component 1054. The inner diameter of the third annular section 1514 may be the same as the second inner diameter of the second annular section 1512. The distal end of the fourth annular section 1516 may be aligned with the proximal end of the second securement component 1054. The fifth annular section 1518 may be positioned above the proximal ends 1474 of the basket splines 1442. The proximal end of the sixth annular section 1520 is proximal to the ends of the basket splines 1442.

The distal end of the shaft 1606 may extend to the proximal end of the second annular section 1512 of the retention ring 1502. The distal end of the shaft 1606 may abut the distal shaft electrode 1610a or the second annular section 1512 of the retention ring 1502. The shaft 1606 may be secured to the retention ring 1502 by adhesive and/or welding. A benefit of this arrangement is that the retention ring 1502 and the shaft 1606 overlap which may improve catheter performance.

The electrode(s) coupled to the retention ring may be ring electrodes. As shown in FIG. 16, the distal shaft electrode 1610a is positioned over the third annular section 1514. The blunt proximal end of the second annular section 1512 may function as a hard stop for the distal shaft electrode 1610a. The distal shaft electrode 1610a may overmolded to the retention ring 1502. A benefit of utilizing the retention ring 1502 is that the distal shaft electrode 1610a does not require swaging because and the distal shaft electrode 1610a is positioned against the blunt proximal end of the second annular section 1512 which may also function as a hard stop. As illustrated in FIG. 16, a second shaft electrode 1610b is positioned proximal to the retention ring 1502. One benefit illustrated in FIG. 16 is that the shaft electrodes 1610 may be narrower than typical shaft electrodes. This provides for narrower spacing between the distal shaft electrode 1610a and the adjacent shaft electrode 1610b.

One benefit of the coupler 1002 is shown in FIG. 16, is that because the sensors 1408 are located within the lumen of a balloon coupled to the proximal coupler 1002, the distal shaft electrode 1610 may be positioned closer to the end assembly where the sensor(s) may typically be located. With this arrangement, information about the position and/or orientation of the end assembly determined from signals from the distal shaft electrode 1610a may be obtained faster than for a more proximally located distal shaft electrode. Additionally, an end assembly with this proximal coupler 1002 is shorter which may improve the function of the catheter, e.g., easier navigation through the introducer.

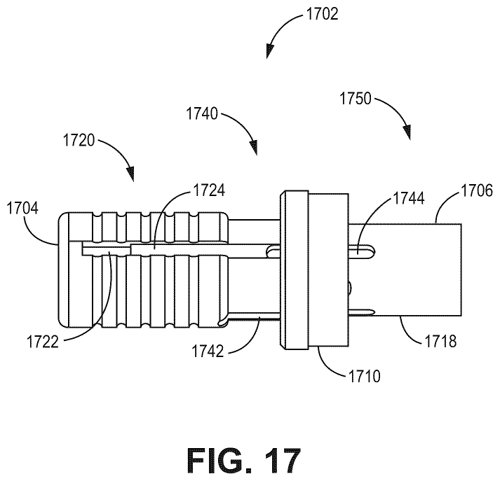

FIG. 17 is an isometric view of another embodiment of a proximal coupler 1702. Proximal coupler 1702 has the same benefits as proximal coupler 1002. The proximal coupler 1702 includes a coupling component 1718 with a distal end 1704 and a proximal end 1706 and a retention ring 1710 positioned around the coupling component 1718. The coupling component 1718 includes a securement region 1720 configured for attaching a balloon to the proximal coupler 1702, an intermediate section 1740 extending from the securement region 1720 to distal end of the retention ring 1710, and a proximal region 1750 extending from the distal end of the retention ring 1710 to the proximal end 1706. The coupling component 1718 further includes at least one groove 1724 and/or at least one slot 1742. Like the proximal coupler discussed above, the at least one slot 1742 may be configured to hold a sensor (e.g., a magnetic sensor). In the view provided in FIG. 17, the groove 1724 is oriented parallel to the longitudinal axis of the coupling component 1718. Like the proximal coupler 1002, the proximal coupler 1702 may include a second groove that is positioned 180° from groove 1724 and oriented at an oblique angle to the longitudinal axis of the proximal coupler 1702. A first opening 1722 may extend distally from the groove 1724. A second opening 1744 extends proximally from the proximal end of the groove 1724. The coupling component 1718 may further include a third opening, e.g., slot 1742. In at least one embodiment the second opening 1744, the third opening 1742 and an additional opening not visible in the view provided in FIG. 17 are configured to route wires, such as magnetic sensor wires, from the outer surface of the coupler to the inner surface of the proximal coupler, under the retention ring 1710 and the back to the outer surface of the proximal coupler proximal to the retention ring 1710.

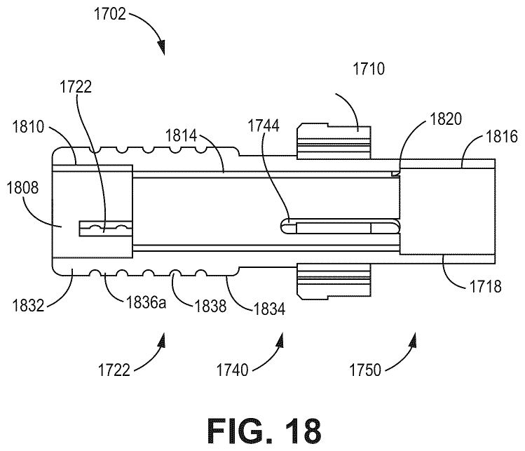

FIG. 18 is a longitudinal cross-section of the proximal coupler 1702 showing the lumen 1808 defined by the coupling component 1718. Like the first securement region 1030 of the proximal coupler 1002 discussed above, the securement region 1720 includes a plurality of ribs that include a distal side rib 1832, at least one middle rib 1836a (collectively 1836), and a proximal side rib 1834. As discussed above, the ribs provide an increased surface area for attaching a balloon. As shown in FIG. 18, the surfaces 1838 between adjacent ribs are curved. As discussed above this increases the surface area compared to a flat surface. In other embodiments, the surfaces between adjacent ribs are flat. In other embodiments, first securement region 1030 may lack ribs and have an overmolded portion as discussed above for the distal coupler embodiments.

The inner surface of the proximal coupler 1702 defines at least one bore. In the embodiment shown in FIG. 18, the proximal coupler 1702 includes a distal bore 1810 with a first diameter sized to receive a basket support shaft, an intermediate bore 1814 with a second diameter less than the first diameter, and a proximal bore 1816 with a third diameter greater than the second diameter and sized to receive a catheter shaft. In some embodiments, the support shaft is free floating in the lumen of the proximal coupler 1702 so that the support shaft may translate (↔ in the view provided) in the distal bore 1810. As discussed above, the support shaft may be bonded to the distal coupler, thus, during use, the relative positions of the distal and proximal couplers may change-further apart or closer together (e.g., during introduction and/or withdrawal of the catheter through the introducer, or during balloon inflation). A catheter shaft inserted into the proximal bore 1816 may abut edge 1820. The catheter shaft inserted into the proximal bore 1816 defines an inflation lumen configured to transport balloon inflation media. In some embodiments, the inflation lumen exits one or more holes in a support tube extending distally from the proximal coupler. In other embodiments, the proximal coupler 1702 includes one or more holes in fluid communication with the inflation lumen (not shown).

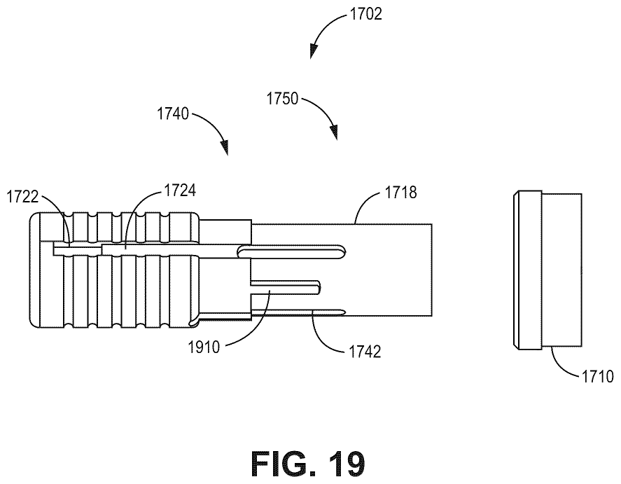

FIG. 19 is an exploded view of the coupling component 1718 and the retention ring 1710. In at least one embodiment, the coupling component 1718 and the retention ring 1710 are coupled together. For example, the coupling component 1718 and the retention ring 1719 are coupled by a groove 1910 formed in the outer surface of the coupling component 1718. Groove 1910 may function as a keyseat.

FIGS. 20A and 20B are isometric and end views, respectively, of the retention ring 1710. The retention ring 1710 includes a wall 2008 defining a lumen 2002, a first annular section 2012 with a first outer diameter, and a second annular section 2014 with a second outer diameter. In this embodiment, the first outer diameter is greater than the second outer diameter. The wall 2008 defines a plurality of passageways 2016 configured to receive basket splines. The inner surface of the wall 2008 may define a groove 2018 for coupling the retention ring 1710 and the coupling component 1718 together. Groove 2018 may function as a keyway.

Turning to FIGS. 53-59, additional features of a proximal coupler are illustrated. FIG. 53 illustrates a proximal coupler 5302 coupled to at least one shaft, an inner shaft 5308 extending proximally from the proximal coupler 5302. The proximal coupler 5302 includes a coupler body 5306 that may be unitary or formed of a plurality of components. Like proximal coupler 1702, the proximal coupler 5302 includes a distal section 5310, an intermediate section 5340, and a proximal section 5350. The distal section 5310 may be a separable from the intermediate section 5340. For example, the distal section 5310 may include a male connector configured to be received within a central lumen of the intermediate section 5340. The distal section 5310 and the intermediate section 5340 may include complementary features configured to prevent rotation of one section 5310, 5340 relative to the other section 5310, 5340. For example, the complementary features may be a slot and projection. The distal section 5310 may include a balloon securement region. The balloon securement region may be an overmolded region of the distal section 5310. The intermediate section 5340 may include a basket securement assembly that includes a plurality of alternating projections 5312 and grooves/slots 5318. Each projection 5312 may include a distal end 5314 and a proximal end 5316. As discussed above, the projections 5312 and slots 5318 may be configured to secure splines to the proximal coupler 5302 and may also include alignment and/or retention features. The proximal end/edge of the proximal coupler 5302 may define at least one notch, such as notch 5354. The notch 5354 may contain adhesive for securing the proximal coupler 5302 to another component of the catheter, e.g., a catheter shaft.

The proximal coupler may define at least one sensor channel. A sensor channel may be configured to hold an electromagnetic sensor. As illustrated in FIG. 53, the proximal section 5350 defines a sensor channel 5352. The sensor wires 5332 may extend out of the distally oriented end of the sensor 5330. The polarity of sensor 5330 is opposite to the polarity of the sensor 4966 coupled to the distal coupler 4840. Thus, sensor 5330 may be described as having a reversed polarity. A navigation and/or imaging protocol (e.g., software) may be adjusted to accommodate a reversed polarity sensor. The pin connection of the sensor wires 5332 of a reversed polarity sensor may also be adjusted to accommodate a sensor with reversed polarity. A reversed polarity sensor may provide strain relief to the sensor wires 5332. Reversing the polarity may provide enough space for the sensor wires to be routed which may reduce wear and tear. Utilizing the sensor channel for holding the sensor and for routing the sensor wire routing reduces the length needed to route the sensor wires. This may provide for a shorter deflectable pocket. As used herein, a deflectable pocket is a deflectable region that extends between the end of the basket and the deflecting portion of the deflectable shaft.

Turning to FIG. 54, the proximal coupler 5402 may include a retention ring 5419 positioned around the coupler body. The retention ring 5419 may be positioned around splines coupled to the basket retention assembly of the proximal coupler 5402. Thus, the retention ring 5419 may secure a plurality of splines 5422 to the proximal coupler 5402. At least one electrode, collectively 5490, may be coupled to the retention ring 5419. The outer surface of the electrode may be flush with the outer surface of the retention ring 5419. FIG. 53 illustrates a first ring electrode 5490a and a second ring electrode 5490b coupled to the retention ring 5419. The ring electrodes 5490 may be secured to the retention ring 5419 without swaging. For example, the ring electrodes 5490 may be compression fit to the retention ring 5419. As discussed above, the retention ring may be formed of multiple components. For example, the first retention ring component 5432 and the second retention ring component 5442 may be separate components rather than being a unitary component configured to receive the ring electrodes 5490.

An outer shaft 5488 may extend proximally from the retention ring 5419. The outer shaft 5488 may surround the proximal section of the proximal coupler 5302 and an inner shaft, such as shaft 5308. A shaft 5480 may extend distally from the distal section 5410 of the proximal coupler 5402. The shaft 5480 may be the inner shaft, such as shaft 5308, or a separate support shaft, such as support shaft 383 discussed above.

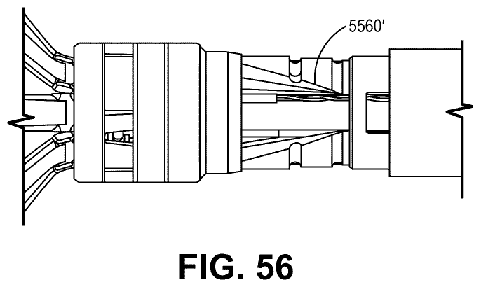

As discussed above, the proximal coupler may define one or more channels or slots. A channel/slot may be configured to route one or more wires. For example, as illustrated in FIG. 54, the proximal coupler 5402 defines a channel 5560 with a wire 5554 routed by the channel 5560. The one or more channels may be defined by the proximal section 5550. The channel/slot may be longitudinally oriented. The channel/slot illustrated in FIG. 55 has straight side walls which provides the channel with a constant circumferential width. However, the side walls may be stepped which provides the channel with a variable circumferential width (see e.g., channel 5560′ illustrated in FIG. 56). A stepped side wall may have a wider width along a first surface of the coupler and a narrower width along a second surface of the coupler For example, a channel with a variable circumferential width may have a funnel shape with a wider width at the distal end of the slot and a narrower width at the proximal end of the slot (see e.g., channel 5560′ illustrated in FIG. 56). This configuration may considerably reduce the tacking of wires extending proximally from the expandable end assembly.

One or more wires may also extend along the outer surface of the proximal coupler 5402. For example, FIG. 55 illustrates a wire 5556 extending under the retention ring 5419 and along an outer surface of the proximal section 5550. The wire 5556 may be coupled to a sensor or an electrode coupled to a component of the end assembly (e.g., basket, distal coupler).

As discussed above, the proximal coupler may include one or more alignment and/or retention features. For example, as illustrated in FIG. 55, the proximal coupler 5402 may include an alignment feature configured to couple the first and second retention ring components 5432, 5442 together. The alignment feature is a key assembly 5538. The first retention ring component 5432 includes the key 5539 and the second retention ring component 5442 defines a slot/notch 5540 configured to receive the key 5539. As another example, the alignment/retention feature may be an edge. For example, as illustrated in FIG. 55, the retention ring 5419 includes a plurality of edges 5534, 5536, 5544 configured to retain/position a plurality of ring electrodes. A ring electrode may be positioned between and/or retained in a recess/groove by one or two edges. The recess/groove may be formed by a section of the retention ring component 5432, 5442 with a reduced outer diameter. For example, the proximal section 5543 of the second retention ring component 5442 may have a reduced outer diameter. As shown in FIG. 55, the first ring electrode 5490a is retained by, and may abut, a first edge 5534 and a second edge 5536. The first edge 5534 may be defined by the first retention ring component 5432. The second edge 5536 may be a distal edge of the second retention ring component 5442. The distal edge of the second ring electrode 5490b may be retained by, and may abut, a third edge 5544. The proximal edge of the second ring electrode 5490b may abut a shaft coupled to the proximal coupler 5302, such as the outer shaft 5488. A retention edge may be provided along the outer surface of the proximal coupler 5302. For example, an edge 5552 extends along the outer surface of the proximal section 5350 of the proximal coupler 5302. The edge 5552 may provide a hard stop for the second retention ring component 5442. An alignment/retention feature may also be provided on the inner surface of the proximal coupler (see e.g., FIG. 57).

Turning to FIG. 57, a cross-section view of the proximal coupler 5402 is provided. The proximal coupler 5402 may include an inner surface 5702 defining a central lumen 5709. As discussed above, the proximal coupler may be formed of a plurality of components. The components of the proximal coupler may overlap. For example, as shown in FIG. 57, the distal side 5738 of the second retention ring component 5442 overlaps the proximal side 5734 of the first retention ring component 5432. Overlapping components may have complementary shapes or surfaces. For example, the shape of the overlapping portion of the proximal section 5543 of the second retention ring component 5442 is complementary to the shape of the portion of the proximal section 5350 that extends distally from the edge 5752. In this example, the complementary shapes are curved surfaces. However, any complementary shapes may be utilized.

As shown in FIG. 57, the basket splines 5422 may be positioned between the retention ring 5419 and the intermediate region 5740 of the proximal coupler 5402. Each spline 5422 may include an electrical connection 5724 coupled to the end of the spline 5422. There may be a space 5716 proximal to the electrical connection 5724. A flex circuit coupled to the electrical connections 5724 may be positioned in the space 5716. The space 5716 may be defined by the inner surface of the second retention ring component 5442 and the intermediate region 5740 of the proximal coupler 5402.

As discussed above, the inner surface of the coupler may include a retention/alignment feature. For example, the proximal coupler 5402 includes an annular projection/edge 5712 positioned along the inner surface of the intermediate section 5340 of the proximal coupler 5302. The projection 5712 may function as a hard stop for the inner shaft 5708, which may abut the projection 5712. Thus, projection 5712 may limit distal movement of the inner shaft 5708.

Wires may be routed along the inner surface 5702 of the central lumen 5709. For example, as illustrated in FIG. 57, wires 5764 may be positioned within a sheath 5762.

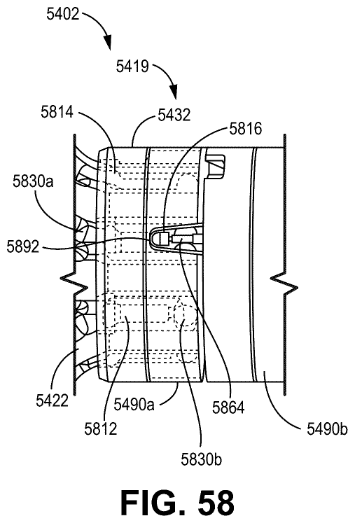

Turning to FIG. 58, features of a retention/alignment feature for a coupler are illustrated. Like the couplers discussed above, the coupler 5402 includes a basket retention assembly with projections 5812 and grooves configured to receive basket splines 5422. As shown in FIG. 58, the splines 5422 may include a first set of spline retention features 5830a configured to abut the distal ends 5814 of the projections 5812 and a second set of spline retention features 5830b configured to abut the proximal ends of the projections 5812. The basket securement assembly may further include a retention/alignment feature configured to couple the intermediate region 5740 of the coupler 5402 to the retention ring 5419. For example, the distal end 5814 of the projection 5812 may include a projection configured to be a hard stop for a distal end of the retention ring 5419. The distal end of the first retention ring component 5432 may define a retention feature that cooperates with the projection to maintain the position of the retention ring 5419. For example, the distal end of the first retention ring component 5432 may extend downward so that it abuts the distal side of the projection. This may limit proximal movement of the retention ring 5419. The distal end of the first retention ring component 5432 may further define an edge that abuts the proximal side of the projection 5812. Thus, the retention feature may be a channel/slot, e.g., defined by the downward extending distal end and the edge. An alignment feature may also be utilized to couple the retention ring 5419 to the coupler body 5306. The alignment feature may include a snap joint comprising a protrusion configured to catch in a depression or slot. For example, the proximal end 5816 of a projection 5812 may be configured to catch in a depression 5892 defined by the inner surface of the first retention ring component 5432.

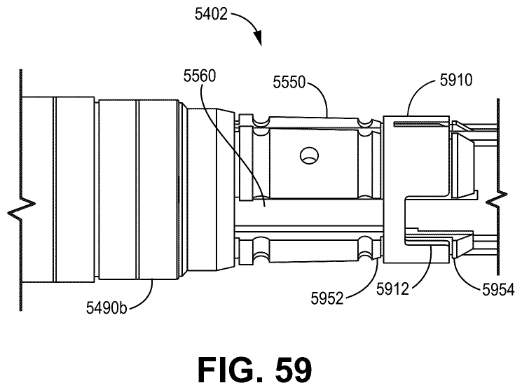

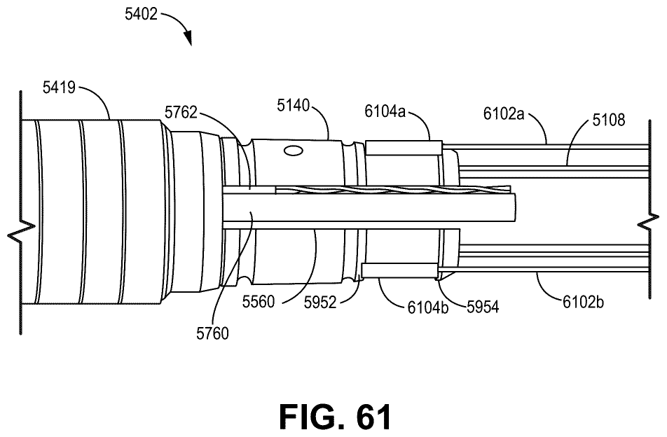

Turning to FIGS. 59-61, the catheter may include a deflection assembly comprising at least one pull wire to deflect the shaft. The deflection assembly may be coupled to the proximal coupler. An advantage of securing the deflection assembly to the proximal coupler, as opposed to a shaft, is that the coupler may provide support for the tensile forces used to deflect the catheter. Another advantage of this arrangement may be an improvement in the electrical isolation between the deflection assembly and the conductors/wires. This arrangement may also allow for more room to route conductors. Additionally, integrating the deflection assembly may be more cost-efficient than utilizing separate coupler and deflection assembly. The at least one pull wire may also “float” in a lumen defined by the inner and outer shafts of the catheter. The pull wire may be attached to the proximal coupler in any suitable way. For example, the pull wire may be secured to the proximal coupler by an anchor. An anchor may secure at least one pull wire to the proximal coupler. Examples of an anchor include a pull wire ring (see e.g., FIG. 59) or a crimp sleeve (see e.g., FIG. 61).

The proximal coupler 5402 may further include at least one retention feature configured to secure at least one pull wire anchor to the proximal coupler 5402. Thus, the retention features may be described as deflection assembly retention features. FIGS. 59 and 61 illustrate a proximal coupler 5402 with two annular retention features, a distal retention feature 5952 and a proximal retention feature 5954 (not visible in FIGS. 54-55 and 57-58). A recess configured to receive a pull wire anchor may be positioned between the retention features 5952, 5954. As illustrated in FIGS. 59 and 61, the retention features 5952, 5954 are discontinuous. For example, channel 5560 extends through the retention features 5952, 5954. However, the retention features may be continuous. The proximal retention feature 5954 may include a sloped proximal surface extending downwards, e.g., towards the longitudinal axis of the proximal coupler 5402.

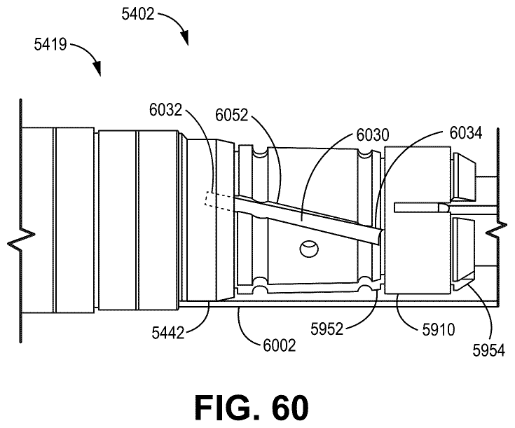

FIGS. 59-60 illustrate different views of a proximal coupler 5402 with a pull ring 5910 positioned between the retention features 5952, 5954. The pull ring 5910 may be swaged, press fit, or adhesively secured to the proximal coupler 5402. The outer surface of the pull ring 5910 may be flush with the outer surfaces of when the pull ring 5910 is positioned between the retention features 5952, 5954. At least one pull wire, not visible in this view, may extend proximally from the pull ring 5910. An outer shaft, such as outer shaft 5488, may be positioned around the pull ring 5910 and abut the ring electrodes second ring electrode 5490b. The pull ring 5910 may define one or more slots (hereinafter a pull ring slot) extending distally from the proximal end of the pull ring 5910. A pull ring slot may be co-located with a slot/channel defined by the proximal coupler 5402. For example, as illustrated in FIG. 59, the pull ring 5910 includes a pull ring slot 5912 co-located with channel 5560. The width of a pull ring slot may be at least as wide as a co-located slot/channel defined by the proximal coupler. For example, the widths of the pull ring slot 5912 and channel 5560 are the same. Providing a pull ring slot having a width at least equal to the width of a slot/channel defined by the proximal coupler may provide space for routing of wires and/or cables in the slot/channel. The pull ring 5910 may lack slots extending proximally from the distal edge.

FIG. 60 illustrates the location and orientation of a magnetic sensor 6030 relative to the pull ring 5910. The magnetic sensor 6030 is positioned in a sensor channel 6052. The magnetic sensor 6030 may be oriented at a oblique angle relative to the longitudinal axis of the proximal coupler 5402. The magnetic sensor 6030 has a distally oriented end 6032 and a proximally oriented end 6034. As illustrated in FIG. 60, the sensor channel 6052 may extend through the distal retention feature 5952. The proximally oriented end 6034 of the magnetic sensor 6030 may abut the pull ring 5910. As discussed above, the sensor 5330 may have a reversed polarity with the sensor wires extending out of the distally oriented end 6032. The distally oriented end 6032 may be positioned under a portion of the retention ring 5419. As illustrated in in FIG. 60, the second retention ring component 5442 of the retention ring 5419 is positioned over the distally oriented end distally oriented end 6032.

FIG. 61 illustrates crimp sleeves (collectively 6104) positioned between the retention features 5952, 5954. A crimp sleeve 6104 may abut one or both of the retention features 5952,5954. A first pull wire 6102a extends proximally from a first crimp sleeve 6004a and a second pull wire 6102b extends proximally from a second crimp sleeve 6004b. The second pull wire 6102b may be positioned opposite (180°) from the first pull wire 6102a.

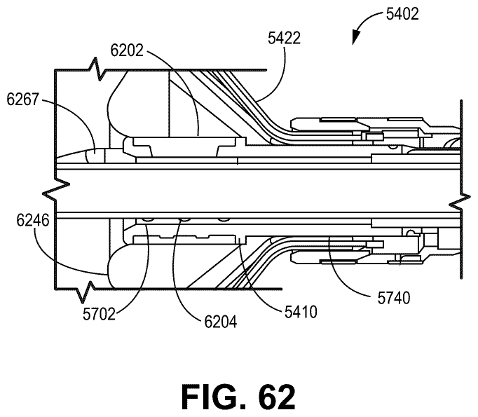

Turning to FIG. 62, additional features of the distal section 5410 of the proximal coupler 5402 are illustrated. As discussed above, the distal section 5410 of the coupler 5402 may include a balloon securement region configured to secure a balloon 6246 to the proximal coupler 5402. The balloon securement region may include at least one through hole 6204 extending from the outer surface of the distal section 5410 to the intermediate region 5740 of the proximal coupler 5402. Thermal bonding may be utilized to couple to the balloon the to the proximal coupler instead of an adhesive bond. The overmold 6202 may be configured for thermal bonding the balloon to the proximal coupler and/or for bonding the distal sensor wires 6267 to the proximal coupler. For example, when the overmold 6202 is heated, the overmold 6202 may reflow into one or more of the through holes 6204 so that at least a portion the overmold 6202 extends through to the inner surface 5702 of the proximal coupler 5402. The durometer hardness of the overmold 6202 may be similar to the balloon durometer hardness for bonding the balloon to the proximal coupler. The material forming the proximal coupler 5402 may be stiffer and/or harder than the material used for the overmold 6202. A laser may be utilized to heat the overmold 6202.

As discussed above, basket splines may be oriented parallel or non-parallel to the longitudinal axis of the end assembly when the basket is in an expanded state. The proximal and distal couplers discussed above may be utilized to couple a basket with basket splines oriented parallel or non-parallel to the longitudinal axis of the end assembly when the basket is in an expanded state.

A medical device, such as catheters 102, 202, 302, may be configured to bend as it is advanced through an introducer and/or a body lumen. A challenge with steerable catheters is predicting how the catheter will bend—it may bend too much or too little. Thus, it would be beneficial to control the curving or bending of the catheter. It may also be beneficial to have a preset curve shape for a catheter configured to be utilized in challenging anatomy. Additionally, the force(s) applied to the distal end of the catheter as the catheter is advanced may be greater than other sections of the catheter. This may affect the performance of the catheter. For example, the shaft may kink. Thus, it would be beneficial to improve the response to applied force(s).

A pattern may be cut into at least one section of a shaft (herein after referred to as a cut shaft section) to control bending of the catheter and/or to control kinking. The pattern may be non-helical or helical. Generally, the pattern is cut into the wall of the shaft so that the cut extends from the outer surface of the shaft to the inner surface of the shaft.

At least one of the shafts of a catheter may include at least one cut shaft section. A cut shaft section may be located inside an end assembly, such as the basket 240 or end assembly 339. For example, a shaft, such as support shaft 382 discussed above. Includes a cut shaft pattern. As another example, an inner shaft, such as inner shaft 308, includes a cut shaft pattern. A cut shaft section may be located inside an end assembly and at least one cut shaft section may be located proximal to the end assembly. It may also be advantageous to have a steerable catheter with two steerable cut shaft sections. For example, the catheter may have a first section configured to bend at a predetermined angle (a 90° angle for example), a second straight section extending from the first section, and a third curved section extending from the second section. Different patterns may be utilized for different sections. For example, the helical pattern for the first section and the third section may be the same helical pattern with different dimensions, or different helical patterns. A catheter with at least one cut shaft section may be utilized for a variety of procedures. For example, electrophysiological procedures, dilatation procedures, and device delivery/implantation procedures (e.g., implantation of a left atrial appendage (LAA) occluder or a stent).

A bent shaft experiences compressive stress along the inside of the curve and tensile stress along the outside of the bend. It would be beneficial to distribute the stress/force from the inside/outside of the bend. In at least one embodiment, a cut pattern as described herein distributes force/stress in a circumferential direction.

Turning to FIG. 21, an isometric view of a non-helical pattern 2104 cut into a basket support shaft 2102 is provided. The non-helical pattern 2104 includes a plurality of slots 2106 oriented perpendicular to the longitudinal axis of the basket support shaft 2102 and a plurality of support members 2110 oriented parallel to the longitudinal axis of the basket support shaft 2102. As illustrated, the slots 2106 and the support members 2110 may alternate in a circumferential direction with longitudinally adjacent slots being circumferentially offset from one another. For example, slot 2106a is longitudinally adjacent to, and circumferentially offset from, slot 2106b. This arrangement of slots 2106 sorts the support members 2110 into groups, with each group aligned along a line parallel to the longitudinal axis of the basket support shaft 2102. For example, support members 2110a and 2110b are part of a group of support members 2110 aligned along a first line parallel to the longitudinal axis. Likewise support members 2110c and 2110d are part of a second group of support members 2110 aligned along a second line parallel to the longitudinal axis.

As discussed above, the cut pattern may be helical. A cut shaft section with a helical cut pattern may have reduced column strength compared to a shaft section lacking a helical pattern. A helical pattern may reduce kinking of the cut shaft section. A helical pattern may affect or control the curve of the cut shaft section. For example, a helical pattern with a larger maximum curve can bend more sharply than a helical pattern with a smaller maximum curve angle. The curve angle may be measured as a change in angle/length. For example, the curve angle for a 180° bend may be 18°/inch while a curve angle for a 90° bend may be 9°/inch.

In contrast to typical cut patterns, the bend characteristics of the cut shaft section are determined and predicted by the cut dimensions of a helical pattern. Therefore, a benefit of a helical pattern as disclosed herein is that it may be cut into catheter shafts made from a variety of materials, including metallic materials and/or polymeric materials. Some non-limiting examples of shaft materials include nitinol, stainless steel, polyether block amide (PEBA), polyethylene terephthalate (PET), polyether ether ketone (PEEK), and/or polyetherimide (PEI).

Because the cut dimensions of a helical pattern determine the bend characteristics of the cut shaft section the performance characteristics of a helical pattern cut into shafts formed of different materials are consistent. For example, a nitinol shaft and a stainless steel shaft with the same helical cut pattern exhibit the same curve/bend characteristics. In contrast, a nitinol shaft and a stainless steel shaft with the same typical cut pattern exhibit the different curve/bend characteristics. The ability to utilize the same helical pattern into shafts made from different materials provides flexibility in design. For example, a helical cut pattern as described herein may be utilized for shafts of catheters configured for different procedures. Additionally, the material selected for the shaft may be made independent from the selection of the helical pattern. Rather the choice of shaft material may be made based on other considerations, e.g., intended use, strength, cost, etc. The helical pattern may be made by laser cutting or milling. In one example, a Swiss machine is used to cut the pattern into the shaft. In some embodiments, the outer surface and/or the inner surface of the cut shaft section is bare—in other words, there is no coating or layer on the surface(s).

Table 1 provides some equations describing some of the characteristics of a helical pattern disclosed herein. Exemplary helical patterns with these attributes are discussed below in greater detail with reference to FIGS. 22-33.

TABLE 1

| Attribute | Characterizing Equation |
|---|---|
| Shaft Outer Diameter (OD) | ID + 2WT |
| Shaft Inner Diameter (ID) | OD − 2WT |
| Shaft Wall Thickness (WT) | (OD-ID)/2 |
| Shaft Circumference (C) | OD × π |
| | Where π = 3.14159 |
| Helical Length (SL) of Cut Pattern | n = number of turns about the |
| | longitudinal axis of the shaft |
| | $n = \frac{tube cut length}{pitch ( P )}$ |
| | SL = n\sqrt{C² + P²} |
| Helix angle (θ) | $θ = {tan}^{- 1} \frac{Circumference}{Pitch}$ |
| Cut Quantity (in inches) Conversion to metric (mm): inches × 25.4 | $\frac{tube length - 6 inches}{cut length ( inches )}$ |

where helical length SL represents the length of the unwound pattern, pitch P is the longitudinal distance between adjacent turns of the helical pattern, and helix angle θ is the angle between a turn of the helical pattern and an axis parallel to the longitudinal axis of the shaft (see angle A7 illustrated in FIG. 22).

The pitch P may affect the bending characteristics of the cut shaft section, the longitudinal characteristics of the cut shaft section, and/or the ratio of cut space to material in the cut shaft section. For example, in some embodiments, a cut shaft section with a smaller pitch requires less force to bend than a cut shaft section with a larger pitch. The pitch P selected for the helical pattern may vary. For example, the pitch P of one helical pattern may be seven times (7×) greater than the pitch P for another helical pattern. As illustrated in Table 1, the selected pitch P effects the number of turns about the longitudinal axis of the shaft, the helical length SL of the cut pattern, and the helix angle. For example, the number of turns n of the helical pattern is inversely proportional to the pitch P of the helical pattern. Thus, the number of turns n decreases as the pitch increases. As another example, the helix angle θ is inversely proportional to the pitch P. As the helix angle θ increases, the pitch P may decrease—in other words, for a given length a helical pattern with a larger helix angle θ has more turns (tighter pitch P) than a helical length with a smaller helix angle θ (looser pitch P). The helical pattern may be a left-handed helix with an oblique helix angle θ. The helix angle θ may control the curve of the cut shaft section when it is bent. An oblique helix angle θ may be equal to or greater than 45° and/or less than 90° (90°>θ≥45°). The helix angle θ for the turns (e.g., such as angle A7 of turns 2213 shown in FIG. 22) may be constant when the shaft is straight—each turn is oriented at the same helix angle θ. The helix angle θ utilized in a helical pattern may vary. For example, a first section of the helical pattern may be oriented at a first helix angle θ₁ and a second section of the helical pattern may be oriented at a second helix angle θ₂. This may provide the cut shaft section with two different bending characteristics.

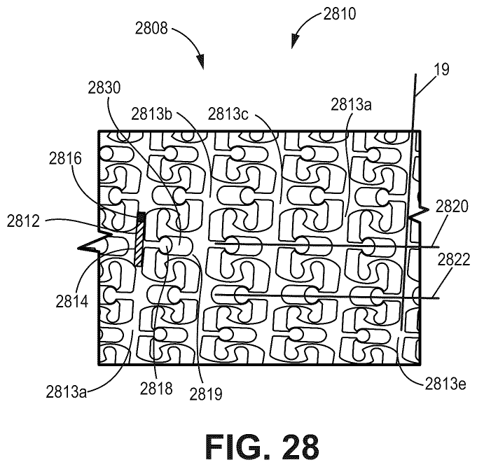

The helical pattern includes a backbone oriented at the helix angle θ. The helically oriented backbone extends in a plurality of turns around the longitudinal axis of the shaft from one end of the pattern to the other end of the pattern. As used herein, a turn extends at most 360° about the longitudinal axis of the shaft. The width of the backbone in different helical patterns may differ. For example, one helical pattern may have a wider backbone than another helical pattern (compare backbone 2212 of FIG. 22 to backbone 2812 of FIG. 28). A helical pattern with a wider backbone may be easier to bend (e.g., requires less force to bend).

Each turn of the backbone may have a plurality of elements. The backbone elements may have the same shape or different shapes. Turns of the helically oriented backbone positioned at the ends of the helical pattern have elements extending from a single side of the backbone while the other turns of the backbone have elements extending from both sides. The backbone may be formed of alternating first sections and second sections where each first section has elements extending therefrom and each second sections has no elements extending therefrom. Elements extending from opposite sides of the first section of the backbone may be aligned along an axis. This arrangement may align the elements of adjacent turns of the backbone into rows. Additionally, when a load is applied to the shaft, the elements may respond to the load like a beam. In contrast, elements of a typical cut pattern that are circumferentially offset or staggered do not respond to a load like a beam. Having a plurality of elements extending from the sides of each turn of the backbone may also improve bending characteristics of the cut section. For example, the cut section may be bendable in multiple directions. Without being bound by theory, a backbone with multiple elements may more easily transition between a tighter pattern (adjacent turns closer together) or looser pattern (adjacent turns father apart) to allow a varied bend angle along the length of the cut shaft section. As another example, the cut section may have different bend angles along the length of the cut shaft section.

The plurality of elements may be interlocking. For example, the elements may include an element base, extending from the backbone, with a first width, and an element head, positioned at an end of the element base, with a second width greater than the first width. One element, or two circumferentially adjacent elements extending from a first turn of the backbone, may define a slot with an open end defined by the element head(s) and sides (width) defined by the element base(s). The slot may be sized to receive an element extending from a second adjacent turn. The element(s) may translate within the slot. For example, the element(s) may translate when the cut shaft section is linear and/or bent. Because the orientation of the slot includes a longitudinal component, the cut shaft section may longitudinally expand/contract when the elements translate within the slots, thereby providing another degree of freedom to the cut shaft section. In other words, the cut shaft section does not resist tensile elongation or compressive shortening. This is in contrast to typical cut patterns which limit longitudinal movement (tensile elongation or compressive shortening) of the cut shaft section. The amount of longitudinal expansion/contraction may be calibrated. For example, the amount of longitudinal expansion/contraction of the helical pattern may be modified to a desired amount by adjusting the size/length of the element base, the size/length of the element head, the helix angle θ, and/or the pitch P. Additionally, as noted above, the longitudinal characteristics of the cut shaft section may be affected by adjusting the pitch which affects the number of turns for a given length. For example, longitudinal expansion/contraction may be greater for a larger pitch than for a smaller pitch.

The helical pattern may be tailored to a desired amount of strength. For example, a cut shaft section with a first helical pattern (such as pattern 2210 illustrated in FIG. 22) may have a greater strength for the same wall thickness and tubing size compared to a cut shaft section with a second helical pattern (such as pattern 2810 illustrated in FIG. 28).

A first embodiment of a helical pattern with at least one of the characteristics/features described above is illustrated in FIGS. 22-27. Turning to FIG. 22, a left-handed helical pattern 2210 cut through the wall of a shaft 2208 is illustrated. The pattern 2210 includes a backbone 2212 that extends helically around the longitudinal axis of the shaft 2208 for at least one turn with nine turns 2213a-2213i (collectively turns 2213) illustrated in this view. The number of turns 2213 may depend on the helical length SL of the helical pattern and/or the helix angle θ which are discussed above. As illustrated, the backbone 2212 may be symmetrical about a long axis 12. The long axis 12 may bisect the backbone 2212 into halves that are mirror images. When the shaft 2208 is straight, as shown in FIG. 22, each turn 2213 is oriented at an oblique helix angle A7 that is measured between an axis 12 of the backbone 2212, provided for turn 2213i in this illustration, and an axis 10 parallel to the longitudinal axis of the shaft 2208. A plurality of elements, discussed below in greater detail, extend from the sides of the backbone 2212.

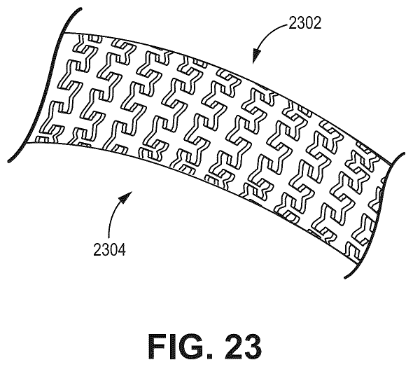

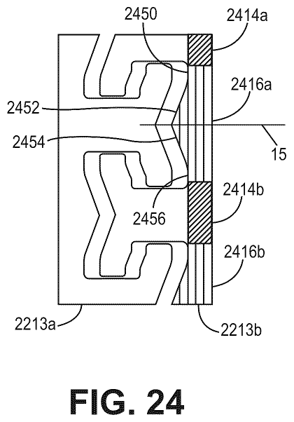

A gap or slot 2230 between turn 2213c and turn 2213d may define the geometry of the backbone 2212 and the elements extending from the backbone. The gap 2230 may have a uniform width. For example, in some embodiments, the gap 2230 may have a uniform width when the shaft is straight, as illustrated in FIG. 22. The gap 2230 may have a non-uniform width. For example, the gap 2230 may have a non-uniform width when shaft is bent, as illustrated in FIG. 23. The gap 2230 may have a non-uniform width when the shaft is straight, as illustrated for example in FIG. 24. The pattern 2210 may include rounded edges/corners which may provide greater strength and/or durability than square edges/corners.

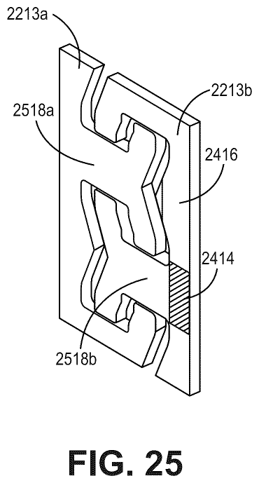

As discussed above, the cut shaft section may control bending of the catheter. FIG. 23 illustrates an example of how the helical pattern 2210 may adapt to a bend in the shaft 2208. A benefit of helical pattern 2210 is that it can adapt to a greater variety of curves compared to a typical cut pattern. The helical pattern 2210 may experience compressive stress along the inside 2304 of the curve and tensile stress along the outside 2302 of the bend. Features of the helical pattern 2210, such as element 2518 shown in FIG. 25, may translate in a longitudinal direction and/or rotate in a circumferential direction as the cut shaft section bends. As illustrated in FIG. 23, adjacent turns 2213 are compressed (closer together) along the inside 2304 of the bend, compared to along the outside 2302 of the bend. This performance feature may be provided by the alignment of the elements 2518, as discussed below. In other words, along the inside of the curve the element 2518 may abut the adjacent turn 2213 of the backbone 2212 while along the outside 2302 of the curve there may be a gap between the elements 2518 and the adjacent turn 2213 of the backbone 2212. The pitch P between adjacent turns may be larger along the outside 2302 of the bend than the pitch P along the inside 2304 of the bend. The elements 2518 may remain within the wall of the shaft 2208 when the shaft bends. This may provide the shaft 2208 with a smooth outer surface.

Turning to FIG. 24, halves of two adjacent turns 2213a, 2213b of the pattern 2210 are illustrated. The backbone 2212 in this embodiment is formed by alternating backbone sections that have different shapes. Two first backbone sections 2414a, 2414b of turn 2213b are identified in FIG. 24 by a first hashmark pattern and two second backbone sections 2416a, 2416b of turn 2213b are identified by a second hashmark pattern. In this example, the first backbone sections 2414 are shorter than the second backbone sections 2416. As illustrated in FIG. 24, the elements of the helical pattern extend only from the first backbone sections 2414. The first backbone section 2414 and/or the second backbone section 2416 may be symmetrical about an axis, such as axis 15, that is perpendicular to the long axis of the backbone 2212. The first backbone sections 2414 may have a constant width while the second backbone sections 2416 have a variable width.

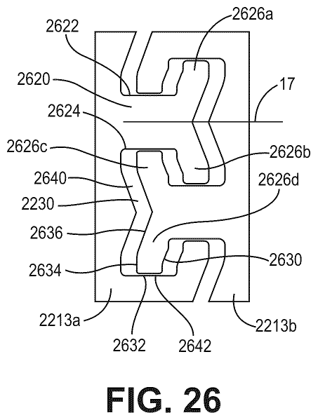

Elements (such as elements 2518 shown in FIG. 25) may be coupled to, or extend from, the side surface of the first backbone sections 2414 while no elements are coupled to, or extend from, the side surfaces of the second backbone sections 2416. As used in this application, a side surface extends from the outer surface of the shaft to the inner surface of the shaft. Each second backbone section 2416 may include a first backbone side surface 2450, a second backbone side surface 2452, a third backbone side surface 2454, and a fourth backbone side surface 2456. The first backbone side surface 2450 extends between a first backbone section 2414a and the second backbone side surface 2452. The second backbone side surface 2452 extends between the first and third backbone side surfaces 2450, 2454 and the third backbone side surface 2454 extends between the second and fourth backbone side surfaces 2452, 2456. The fourth backbone side surface 2456 extends between the third backbone side surface 2454 and another first backbone section 2414b. The second backbone side surface 2452 and the third backbone side surface 2454 are oriented at an obtuse angle to one another (such as angle A8 shown in FIG. 26). The first backbone side surface 2450 and/or the fourth backbone side surface 2456 may be straight or curved.

In helical pattern 2210, the elements extending from the backbone have the same shape. Two elements 2518a, 2518b (collectively elements 2518) extending from adjacent turns 2213a, 2213b of the backbone are illustrated in FIG. 25. Circumferentially adjacent elements 2518 extending from adjacent turns 2213 extend in opposite directions. For example, element 2518a extends in a first longitudinal direction and element 2518b extends in a second longitudinal direction opposite to the first longitudinal direction. Each element 2518 extends from a first backbone section 2414 (one of which is hashmarked) and is positioned adjacent to a second backbone section 2416 of the adjacent turn 2213 of the backbone 2212. Both element 2518a and element 2518b are one of a pair of elements 2518 extending from opposite sides of a first backbone section 2414 (in FIGS. 22-23), while no elements 2518 extend from the sides of the second backbone sections 2416. As best shown in FIG. 22, the elements 2518 are aligned (in a row) and oriented at an oblique angle to an axis 10 parallel to the longitudinal axis of the shaft 2208 when the shaft 2208 is not curved. The alignment of elements 2518 in a row may provide for beam bending and/or reduce twisting as the shaft bends.

Additional features of the elements 2518 are identified in FIG. 26. For example, as illustrated, an element 2518 may be positioned in a slot 2230 with non-uniform gaps 2640, 2642 between the element 2518 in the slot 2230 and the turn 2213a of the backbone and the adjacent elements 2518 defining the slot 2230. The width of the gap 2642 between circumferentially adjacent elements 2518 is less than the width of the gap 2640 between an element 2518 in a slot 2230 and an adjacent turn 2213 of the backbone 2212. A narrow slot 2230 between circumferentially adjacent elements 2518 may reduce the amount the cut pattern 2210 may bend.

The elements 2518 may be symmetrical about a long axis 17. The elements 2518 include a post 2620, a first arm 2626a, and a second arm 2626b (collectively arms 2626), with the post 2620 forming the base of element 2518 and the arms 2626 forming the head of the element 2518. As discussed above, the elements 2518 are interlocking. As shown, the circumferential width of the element head (arms 2626) is greater than the circumferential width of the element base (post 2620) so that the element heads (arms 2626) of circumferentially adjacent elements 2518 extending from one turn (turn 2213b in FIG. 26) of the backbone 2212 define an opening of a slot 2230 sized to receive the element base (post 2620) extending from an adjacent turn (turn 2213a in FIG. 26) backbone 2212. The sides of the slot 2230 are defined by the bases (posts 2620) of the circumferentially adjacent elements 2518. Also, the width of a slot 2230 is equal to the length of a second backbone section 2416, (width and length measured up ↔ down in the view provided in FIG. 26).

The post 2620 extends from the backbone 2212 and includes opposing first and second post side surfaces 2622, 2624 oriented parallel to the long axis 17. The length of the post 2620 may affect the curve angle for the helical pattern, as discussed below in greater detail. As illustrated in FIG. 26, the first and second post side surfaces 2622, 2624 are straight.

The arms 2626 are located at an end of the post 2620 and include a plurality of side surfaces. For example, arm 2626d includes a first arm side surface 2630, a second arm side surface 2632, a third arm side surface 2634, and a fourth arm side surface 2636. The first arm side surface 2630 extends between the post side surface and the second arm side surface 2632. The first arm side surface 2630 extends at an angle from a post side surface 2622, 2624. The first arm side surface 2630 of longitudinally adjacent arms 2626 may have reciprocal shapes so the first arm side surface 2630 may mate/abut when brought into contact with one another. The first arm side surface 2630 in this embodiment includes at least one curve.

The second arm side surface 2632 extends between the first arm side surface 2630 and the third arm side surface 2634. In this embodiment, the second arm side surface 2632 is oriented parallel to the long axis 17 of the element 2518. An element with a second arm side surface, such as element 2518, may be stronger than an element with a corner between the first and third arm side surfaces—in other words an element lacking a second arm side surface (not illustrated).

The third arm side surface 2634 extends between the second arm side surface 2632 and the fourth arm side surface 2636. The third arm side surface 2634 may be generally orthogonal to the second arm side surface 2632. The third arm side surface 2634 may be an extended rounded corner formed by a plurality of straight sections, as illustrated in FIG. 25, or a single straight section as illustrated in FIG. 26.

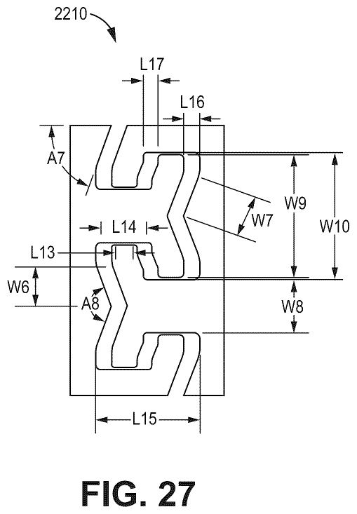

The fourth arm side surface 2636 extends between the third arm side surface 2634 and the fourth arm side surface of arm 2626c. The fourth arm side surfaces 2636 of two arms of an element 2518 may extend at an obtuse angle (such as angle A8 illustrated in FIG. 27). The fourth arm side surfaces 2636 may have reciprocal shapes to the shape of one or more side surfaces of the second backbone section 2416 so the side surfaces may mate/abut when brought into contact with one another. The interaction of the fourth arm side surfaces 2636 of the element 2518 with the second and third backbone side surface 2452, 2454 may improve performance of the pattern 2210 compared to a typical cut pattern. For example, the interaction may maintain the position of the element 2518 relative to the backbone 2212 and/or within the slot 2230.

FIG. 27 illustrates some relationships between features of the pattern 2210, such as angle A7, angle A8, length L13, length L14, length L15, length L16, width W6, width W7, width W8, width W9, and width W10. Angle A7, measured between the second or third backbone side surfaces 2452, 2454 relative to a long axis, such as axis 17 shown in FIG. 26, of the element 2518, is the orientation of the side surfaces 2452, 2454 of the second backbone section 2416. Angle A7 may be an oblique angle. For example, angle A7 may be about 65°-75°. Angle A8, measured between the fourth arm side surfaces 2636 of the arms 2626 of an element 2518 and between the fourth arm side surfaces 2636 of the arms 2626, is the angle between the side surfaces 2452, 2454 of the second backbone section 2416. Angle A8 may be an obtuse angle. For example, angle A8 is about 135°-145°. Width W8, measured between the first and second post side surfaces 2622, 2624, is the width of the post 2620 (the element base). Angle A8 and width W8 may be calibrated to set the location of the load being placed onto the cut shaft section when it is bent. For example, the selection of angle A8 and width W8 may provide for more or less stress placed the inside/outside of a bent cut shaft section. For example, decreasing the width W8 may increase the resistance of the post 2620 to a load applied laterally to the long axis of the post 2620. As another example, increasing angle A8 may increase the circumferential distribution of the load. Distributing the loading so that it is not concentrated along the inside/outside of the bend may increase the strength of the cut shaft section. A more distributed loading also provides design flexibility. For example, the helical cut pattern 2210 may be incorporated into shafts of catheters used for different procedures.

Length L13, measured from the first arm side surface 1530 and the third arm side surface 1534, is the length of the second arm side surface 2632. Length L14, measured from the side of the backbone 2212 to the arms 2626, is the length of the post side surfaces 2622, 2624. Length L14 may be greater than length L13 (L14>L13). As noted above, the length L14 of the post 2620 affects the maximum curve angle of the helical pattern 2210. For example, a larger length L14 provides the cut shaft section with a larger maximum curve angle. Additionally, modifying length L13 or length L14 may provide the helical pattern 2210 that is looser or tighter. For example, decreasing length L13 and/or L14 may provide a tighter helical pattern (more turns per length of shaft). Modifying length L13 or length L14 may also provide for a varied bend angle along the length of the cut shaft section. For example, length L14 could be decreased or length L13 may be increased to create a more gradual curve when the cut shaft section is bent. Length L13 and/or length 14 may be constant. In other embodiments, length L13 and/or length L14 are variable. For example, elements extending from one turn of the backbone may have a first length L13 and a first length L14 and elements extending from another turn of the backbone may have a second length L13 and a second length L14.

Elements 2518 extending from adjacent turns 2213 of the backbone 2212 may translate relative to one another in a generally longitudinal direction. For example, in this embodiment, the elements 2518 may move within slots 2230 defined by two circumferentially adjacent elements 2518. When cut shaft section is subject to compressive shortening, the element 2518 may abut the backbone 2212 of the adjacent turn 2213 so that there is a gap between the head of the element 2518 in the slot 2230 and the heads of the elements 2518 defining the opening to the slot 2230 (e.g., gap between arm 2626b and arm 2626c). The amount of longitudinal translation may be represented by the difference between length L13 and length L14 (L14−L13). The length of L14−L13 also represents the maximum length of the gap between arms 2626b and 2626c when the element 2518 in the slot 2230 abuts the backbone 2212 under compressive shortening and the maximum length of the gap between the head (e.g., arms 2626) of the element 2518 in the slot 2230 and the adjacent turn 2213 of the backbone 2212 under tensile elongation. Modifying length L14 and/or length L13 may affect the longitudinal expansion/contraction of the cut shaft pattern. For example, longitudinal expansion/contraction may be increased by increasing length L14 and/or decreasing L13.

Length L15 is measured between first and fourth backbone side surfaces 2450, 2454 of second backbone sections 2416 of adjacent turns 2213 of the backbone 2212. Length L15 represents the maximum length for the pitch P between adjacent turns of the backbone.

Length L16 is the longitudinal width of the gap 2230 between a turn 2213 of the backbone 2212 and an element 2518 extending from an adjacent turn 2213 of the backbone 2212 when the element 2518 is positioned in the middle of the slot. Length L17 is the longitudinal width of the gap between elements 2518 extending from adjacent turns 2213 of the backbone 2212. When the element 2518 is in this position, length L16 and length L17 may both equal (L14−L13)/2. The lengths of length L16 and length L17 are reciprocal. When the arms of the element 2518 abut the backbone 2212, length L16 is about zero and length L17 is at its maximum. Likewise, when the arms of circumferential adjacent elements 2518 abut, length L17 is about zero and length L16 is at its maximum. The maximum value of length L16 and length L17 depends on the magnitude of lengths L13, L14, and/or L15. In some embodiments, the maximum value for length L16 and length L17 may equal the difference between length L14 and length L13 (L14−L13).

Width W6, measured between a longitudinal line extending from the end of the fourth arm side surface 2636 and a longitudinal line extending from the beginning of the third arm side surface 2634, is the circumferential length/extent of the fourth arm side surface 2636. Width W7, measured from the fourth arm side surface 2636 of an adjacent arm of the element 2518 to the third arm side surface 2634, is the length of the fourth arm side surface 2636. Width W7 may be greater than width W6. Width W9, measured between the two second arm side surfaces 2632 of an element 2518, is a cumulative width of the arms 2626a, 2626b and represents the width of the head of the element 2518. As discussed above, width W9 is greater than the width W8 (W9>W8; i.e., width of element head>width of element base). Width W9 may be 2-3 times greater than width W8. Width W9 may be 2.5 times greater than width W8. Width W10, measured between post side surfaces 2622, 2624 of circumferentially adjacent posts 2620 extending from the side of the same turn 2213 of the backbone 2212, is a circumferential gap between circumferentially adjacent elements oriented in the same longitudinal direction. The relative lengths of widths W9 and W10 may be configured for translation and/or rotation of elements 2518 during bending of the shaft. Width W9 may be at most 98% of width W10. Width W9 may be at least 60% of width W10. In at least one embodiment, A8>A7, W10>W9>L15>W8>L14>W7>W6>L13, and L16 and L17 are variable.

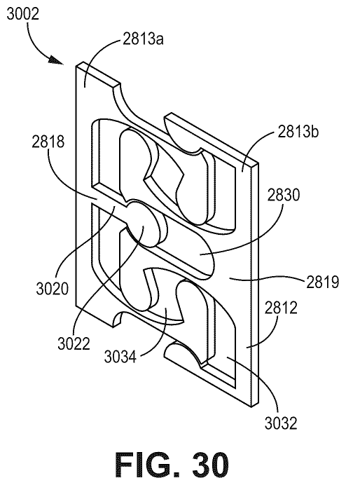

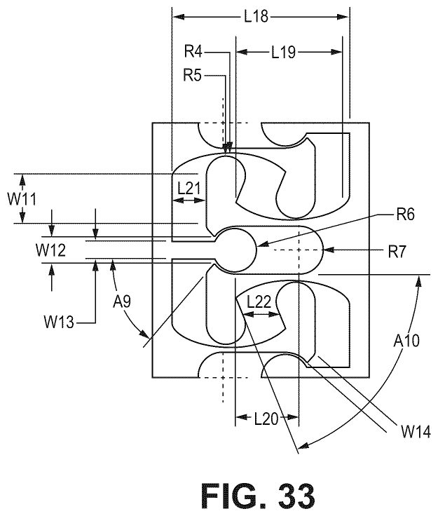

A second embodiment of a helical pattern with one or more characteristics/features described above is illustrated in FIGS. 28-33. In contrast to helical pattern 2210, the elements of helical pattern 2810 have different shapes. Enlarged views of a portion of the pattern 2810 cut into the wall of the shaft 2808 are provided in FIGS. 30-33 with some measurements of the pattern 2810 identified in FIG. 33. As discussed above, an advantage of the helical pattern 2810 is that the performance of the shaft during use does not rely on the material characteristics of the tubing.

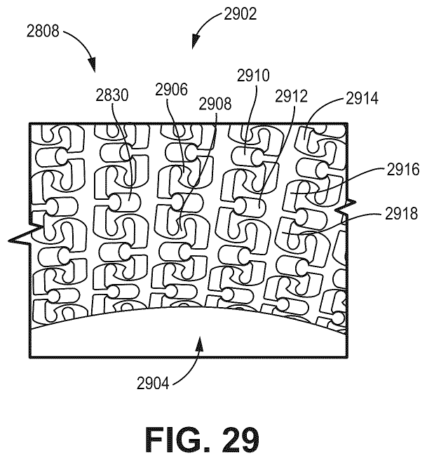

As shown in FIGS. 28 and 29, the helical pattern 2810 is a left-handed helical pattern cut into a section of the shaft 2808. The helical pattern 2810 includes a backbone 2812 extending around the longitudinal axis of the shaft for a plurality of turns 2813a-e (collectively turns 2813), a plurality of first elements 2818 with a first shape, a plurality of second elements 2819 with a second shape, and a plurality of slots 2830. The backbone 2812 includes alternating first backbone sections 2814 (identified by a first hash marking pattern in FIG. 28) and second backbone sections 2816 (identified by a second hash marking pattern in FIG. 28). Like the helical pattern 2210, the second backbone section 2816 is shorter than the first backbone section 2814 and the elements 2818, 2819 extend only from the first backbone sections 2814. In this example, the backbone 2812 is symmetrical about a long axis 19 and has a consistent width measured perpendicular to the long axis 19. The backbone 2812 is oriented at an oblique angle to the longitudinal axis of the shaft 2808, such as the angle between long axis 19 and line 2820.

Like the elements 2518 of helical pattern 2210, the first and second elements 2818, 2819 of helical pattern 2810 are configured to be interlocking with the first element 2818 positioned in a slot 2830 defined by the second element 2819. In this embodiment, the first elements 2818 and the second elements 2819 alternate along each side of the backbone 2812. A first element 2818 and a second element 2819 extend from opposite sides of the same section of the backbone 2812. This arrangement produces longitudinal rows of alternating first and second elements 2818, 2819 where the elements 2818, 2819 of circumferentially adjacent rows are oriented in opposite directions. For example, for the row along line 2820 the first and second elements 2818, 2819 alternate with the first elements 2818 oriented in a first direction (to the right of the figure) and the second elements 2819 oriented in an opposite second direction (to the left of the figure). Similarly for the row along line 2822, the first and second elements 2818, 2819 alternate with the first elements 2818 oriented in the second direction and the second elements 2819 oriented in the first direction. As illustrated in FIG. 28, the long axis of the first element 2818 may be aligned with the long axis of the slot 2830 when the cut shaft section is not bent (linear).

As discussed above, the cut shaft section may control bending of the catheter. FIG. 29 illustrates how the helical pattern 2810 adapts to a bend in the shaft 2808. A benefit of helical pattern 2810 is that it can adapt to a greater variety of curves compared to a typical cut pattern. In at least one embodiment, the first and second elements 2818, 2819 may translate and/or rotate as the shaft bends. For example, the position of the elements 2818 in the slots 2830 may differ. For example, because the first elements 2818 may translate in the slot 2830, at the outside 2902 of the curve/bend, the first elements 2818 are positioned adjacent to the opening of the slot 2830 while at the inside 2904 of the curve/bend the first elements 2818 are positioned away from the opening of the slot 2830. Also, because the first elements 2818 may rotate, the orientation of first elements 2818 may vary when the shaft bends. For example, the 2818 may be closer to one side of the slot than to the other. Thus, pattern 2810 can accommodate tensile elongation and compressive shortening when the cut shaft section is straight and/or bent.

In addition to the slot 2830, the helical pattern 2810 may define one or more additional gaps or openings. Translation and/or rotation of the elements may affect the gap(s) between adjacent elements 2818, 2819. Gaps between adjacent elements may be smaller along the outside 2902 of the bend than along the inside 2904 of the bend. For example, gap 2906 is smaller than gap 2908. Gaps between adjacent elements may be larger along the outside 2902 of the bend than along the inside 2904 of the bend. For example, gap 2910 is larger than gap 2912. The size of the gap may progressively decrease from the outside 2902 of the bend to the inside 2904 of the bend. For example, gaps 2914, 2916, 2918 are progressively smaller. The elements 2818, 2819 may remain within the wall of the shaft 2808 when the shaft bends. This may provide the shaft 2208 with a smooth outer surface.

Turning to FIGS. 30-33, additional details of the helical pattern 2810 cut into the wall of the shaft 2808 are illustrated with FIG. 33 identifying some measurements for features of the first and second elements 2818, 2819. FIG. 30 illustrates two adjacent turns 2813a, 2813b the helical pattern 2810 cut into the wall 3002 of the shaft 2808. Each first element 2818 includes a stem 3020 with an end 3022. As illustrated, the diameter of the end 3022 may be greater than the width of the stem 3020. The end 3022 is positioned in the slot 2830. Although, the end 3022 in this embodiment is rounded, the end 3022 may have other shapes. As discussed above, the pattern 2810 may include gaps in addition to the slot 2830. For example, there may be a gap 3032 between a second element 2819 and the backbone 2812 of the adjacent turn 2813 (see also gaps 2914, 2916, 2918, identified in FIG. 29). Additionally, second elements 2819 extending from adjacent turns 2813a, 2813b may define a gap 3034 (see also gaps 2906, 2908 identified in FIG. 29). Rotation and/or translation of the backbone 2812 and/or one or more elements 2818, 2819 may affect the size of gap 3032 and/or gap 3034.

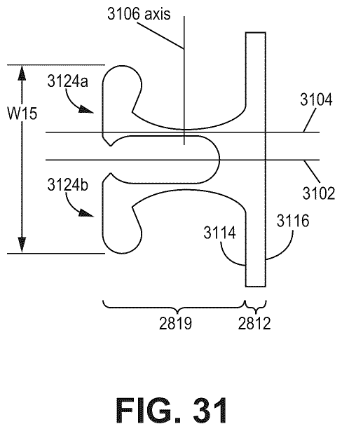

Turning to FIG. 31, additional features of the second element 2819 and the backbone 2812 are illustrated. The backbone 2812 has a first side surface 3114 and a second side surface 3116. The head of the second element 2819 has a width W15. As can be seen in FIG. 28, the base of the second element 2819 has a width substantially equal to the length of the first section 2814 of the backbone. Each second element 2819 may include two members 3124a, 3124b (collectively members 3124). The members 3124a, 3124b of a second element 2819 may be oriented in opposite directions and/or be mirror images about a long axis 3102 of the second element 2819. The member 3124 may be asymmetrical about the long axis 3104 of member 3124 and/or about an axis 3106 perpendicular to the long axis 3104.

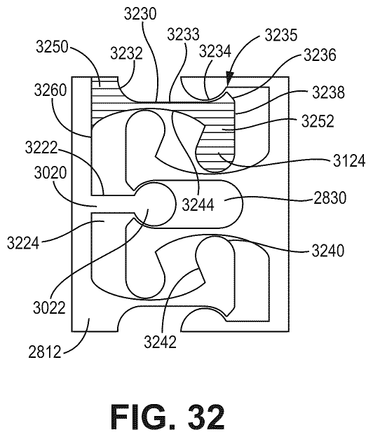

FIG. 32 illustrates additional features of the first elements 2818 and the second elements 2819. The stem 3020 has a length measured from the side 2360 of the backbone 2812 to the rounded end 3022. The stem 3020 of the first element 2818 includes stem side surfaces 2322, 2324 extending from the side 3260 of the backbone 2812. At least the rounded end 3022 is positioned within a slot 2830.

One of the two members 3124 in FIG. 32 is identified by hash marking. Each member 3124 may include a plurality of side surfaces (collectively member side surfaces): a first member side surface 3230, a second member side surface 3236, a third member side surface 3238, a fourth member side surface 3240, a fifth member side surface 3242, and a sixth member side surface 3244. As illustrated in FIG. 32, the first member side surface 3230 includes a straight section 3233 extending between a first curved section 3232 and a second curved section 3234. However, section 3233 may be curved. The first member side surface 3230 is positioned a distance from the backbone 2812. First curved sections 3232 of the members 3124a, 3124b of a second element 2819 may form the closed end of the slot 2830.

The second member side surface 3236 extends from the second curved section 3234 of the first member side surface 3230 to the third member side surface 3238. The edge 3235 between the first member side surface 3230 and the second member side surface 3236 may be rounded. The edges 3235 of the members 3124 of the second element 2819 define the opening of the slot 2830. The distance between the edges 3235 defining the opening of the slot 2830 may be selected so that the first element may be in different orientations. For example, the first element 2819 may be aligned with the slot 2830 or at an oblique angle to the slot 2830. As illustrated in FIGS. 28 and 32 the long axis of the first element 2819 is aligned with the long axis of the slot 2830. As illustrated in FIGS. 28 and 29, the long axis of the first element 2819 is aligned with the long axis of the slot 2830.

The third member side surface 3238 has a reciprocal shape to the shape of the side of the backbone 2812. For example, in this embodiment, both the third member side surface 3238 and the side of the backbone 2812 are straight. When the shaft is bent, the orientation of the third member side surface 3238 to the backbone may vary. For example, for the bend illustrated in FIG. 29, the third member side surface 3238 may be oriented at an oblique angle to the backbone 2812. The fourth member side surface 3240 extends from the third member side surface to the fifth member side surface 3242. The fifth member side surface 3242 extends from the fourth member side surface 3240. The sixth member side surface 3244 extends from the fifth member side surface 3242 to the backbone 2812. As illustrated in FIG. 32, the sixth member side surface 3244 is curved. However, the sixth member side surface 3244 may be straight. There may be a gap between the sixth member side surface 3244 and the fourth member side surface 3240. The gap may allow the members 3124 of adjacent elements 2819 to translate (right ↔ left in the view provided in FIG. 32) as the shaft is manipulated (advanced, bent) during use.

The member 3124 of a second element 2819 may include a narrower segment interconnecting a first wider segment 3250 extending from the side surface (side surface 3114 or 3116) of the backbone 2812 and a second wider segment 3252 at the other end of the member 3124. The first wider segment 3250 may be described as a member base and the second wider segment 3252 may be described as a member head. As illustrated in FIG. 32, the member head has a greater length (up ↔ down in the view provided) than the member base. As shown, the first wider segment 3250 includes a portion of the first member side surface 3230 and a portion of the sixth side surface 3244 and the second wider segment 3252 includes a portion of the first member side surface 3230, the second member side surface, the third member side surface 3238, the fourth member side surface 3240, the fifth member side surface 3242, and a portion of the sixth member side surface 3244.

FIG. 33 illustrates relationships, such as angles and lengths, between features of the helical pattern 2810 such as angles A9 and A10, radius of curvatures R4, R5, R6, and R7, widths W11, W12, W13, and W14; and lengths L18, L19, L20, L21, and L22. Length L18, measured between the sides of adjacent turns of the backbone 2812, represents the pitch P between adjacent turns 2813 of the backbone 2812. As can be seen, the pitch P of helical pattern 2810 is larger than the pitch P of helical pattern 2210. As discussed above, a helical cut pattern with a smaller pitch P may require less force to bend than a helical cut pattern with a larger pitch P. For this reason, more force may be needed to bend helical pattern 2810 than to bend helical pattern 2210.

The second member side surface 3236 has a length W14 and is oriented at an oblique angle A9 to the radially adjacent post side surface (either post side surface 3222 or post side surface 3224). Angle A9 may be about 45°-55°. The straight section 3233 is oriented at an oblique angle A10 to the fifth member side surface 3242. Angle A10 may be about 63°-73°. The first curved section 3232, forming the curved closed end of slot 2830, has a radius of curvature R7. The second curved section 3234 has a radius of curvature that may be complementary to the radius of curvature R6 of the rounded end 3022 of the first element 2818. The radius of curvature R6 may be smaller than the radius of curvature R7. For example, radius of curvature R6 may be 0.8-0.95 of radius of curvature R7. The third member side surface 3238 has a length W11. The third member side surface 3238 and the backbone 2812 form a gap that has a length L21. The fourth member side surface 3240 has a radius of curvature R5. The fifth member side surfaces 3242 of longitudinally adjacent members 3124 form a gap that has a length L22. As discussed above, the size of the gaps depends on the amount of translation, with length L21 and length L22 having a reciprocal relationship. For example, as length L21 decreases length L22 increases. The radius of curvature R4 of the sixth side surface 3244 may be greater than the radius of curvature R5 of the fourth side surface 3240. In at least one embodiment, A9>A10, L18>L19>L20>W11>W12>W13>W14, and L21 and L22 are variable. As illustrated in FIG. 33, L18>L19>L20>W11>L22>L21>W12>W13>W14.

As shown, the slot 2830 is entirely defined by one (1) second element 2819. However, the slot 2830 may be defined by one second element 2819 and by a portion of the backbone 2812. For example, a portion of the first section 2814 of the backbone 2812 may be positioned between two second elements 2819 to define a slot 2830 (not shown). As discussed above, the slot 2830 is sized to hold the first element 2818. The slot 2830 includes an opening, with width W12 at one end sized to accommodate the first element 2818. Width W12 may be measured between the edges 3235 of the members 3124a, 3124b of the second element 2819. As shown, the width W12 of the opening is greater than the width W13 of the stem 3020 and smaller than the width/diameter 2R6 of the rounded end 3022—in other words 2R6>W12>W13. Additionally, in contrast to the slots 2230 of helical pattern 2210, the width of slot 2230 is less than the length of the first section 2814, as illustrated in FIGS. 28 and 31.

The first element 2818 may translate within the slot 2830 for a distance of length L20 (right ↔ left in the views provided in FIGS. 32-33). FIG. 32 illustrates the helical pattern 2810 in a state of tensile elongation. Length L20 is the maximum distance the first element 2818 may translate within the slot 2830 (translation distance). Length L20 may be measured from the center of the rounded end 3022 positioned at the opening to a point representing the location of the center of the rounded end 3022 when the rounded end 3022 abuts the opposite end of the slot 2830. The translation distance of the first element 2818 may be affected by modifying the length of the stem 3020, length L18, length L20, length L21, and/or the radius of curvature R6 of the rounded end 3022. For example, in this embodiment, length L21 limits the translation of the helical pattern 2810 because L21 is less than the length of the stem 3020 which is less than length L20. Increasing the length L21 would increase the translation distance. As another example, a smaller rounded end 3022 may translate for a greater distance than a larger rounded end 3022.

As discussed above, when the helical pattern 2810 bends, some of the rounded ends 3022 of the first elements 2818 may be positioned at the opening to the slot 2830 while others of the rounded ends 2111 are positioned away from the opening to the slot 2830. Rounded ends 3022 positioned at the opening to the slot 2830 may distribute force to the members 3124 defining the opening to the slot 2830. In other words, an applied force may be distributed circumferentially. Distributing the load around the circumference may increase the strength of the cut shaft section compared to a cut pattern that does not distribute the load in a circumferential direction.

A basket provided at the distal end of a medical device, such as the baskets discussed above, may experience forces during use that may cause a change or shift in the relative positions of the basket splines causing the splines to be closer together than desired and/or further apart than desired. For example, expansion and/or deflation of a balloon positioned inside a basket, such as end assembly 339, may cause the splines to shift relative to one another, which may have an adverse effect on performance. As another example, an increase in the distance between adjacent splines may reduce the efficacy of the delivered therapy. For these reasons, it would be beneficial to provide a means of maintaining consistent spline-to-spline spacing.

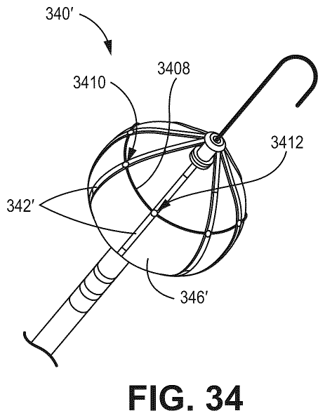

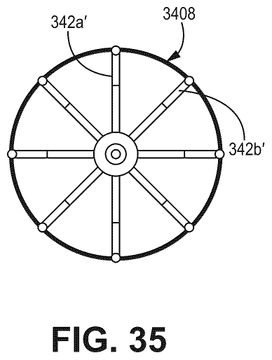

A basket may include tethers 3408, 3608 interconnecting adjacent splines, as illustrated for example in FIGS. 34-36. Although the end assemblies illustrated in FIGS. 34-36, include a balloon 346′, 346″ positioned inside a basket 340′, 340″, a basket without a balloon, such as the end assembly illustrated in FIG. 2, may also include tethers. The tethers may be configured to maintain consistent spline-to-spline spacing of the basket splines despite expansion and/or deflation of the balloon, best shown in FIG. 35. A single tether may extend between each adjacent pairs of splines. The tethers may be positioned between a midpoint of the basket and a distal end of the basket so that the tethers 3408 are visible when the catheter is viewed from the distal end, as shown in FIG. 35. The region between the midpoint and the distal end of the basket may be described as a distal region of the basket.

Each basket spline may include a tether securement feature for coupling an end of a tether. For example, the tether securement feature may be a notch, an eyelet, or a post. The tether may be tied to the tether securement feature. The tether securement feature may be positioned on an exterior surface of the spline, on an interior surface of the spline, or extend from a side of the spline.

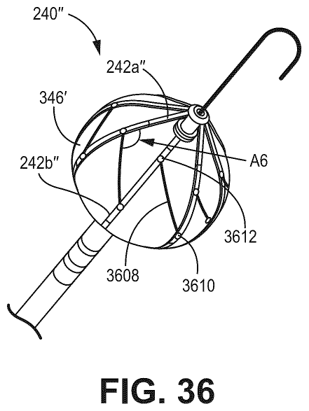

The ends 3410, 3412 of the tethers may be coupled to the adjacent splines so that when the basket is in an expanded state, the tethers are oriented at a perpendicular angle to the splines, as illustrated in FIGS. 34-35. As shown, the ends 3410, 3412 are located at the same longitudinal position along the splines when the basket is in an expanded state and when the basket is in an unexpanded state. The ends 3610, 3612 of the tethers 3608 may be coupled to the adjacent splines 242″a, 242″b so that when the basket 240″ is in an expanded state, the tethers 3608 are oriented at an oblique angle A6 to the splines 242″a, as illustrated in FIG. 36. As shown, the ends 3610, 3612 have different longitudinal positions along the splines when the basket is in an expanded state and when the basket is in an unexpanded state. Orienting the tethers at an oblique angle may reduce the profile of the end assembly during introduction or withdrawal from an introducer.

The tethers may comprise a low elasticity material or an elastic material with a minimum Young's modulus of elasticity. For example, the tethers may have a minimum Young's modules of elasticity of 0.4 GPa. The tethers may be formed of a highly crystalline polymer. Examples of highly crystalline polymers that may be utilized for a tether include polytetrafluoroethylene (PTFE) high-density polyethylene (HDPE), polyethylene terephthalate (PET), and nylon 66.

As discussed above, the end assembly may include a balloon positioned within a basket, such as the end assembly illustrated in FIG. 3. Positioning a balloon within a basket may serve one or more purposes. For example, expanding the balloon so that it contacts the basket may provide mechanical support to the basket. As another example, where the end assembly is being utilized for ablation, a balloon formed of an insulative material may reduce energy loss from electrodes positioned on the basket. A reduction in energy loss may increase therapeutic efficacy of the ablation protocol. For example, larger sized lesions may be generated by reducing the loss of energy. For these reasons, it is helpful to be able to assess expansion state of the balloon relative to the basket.

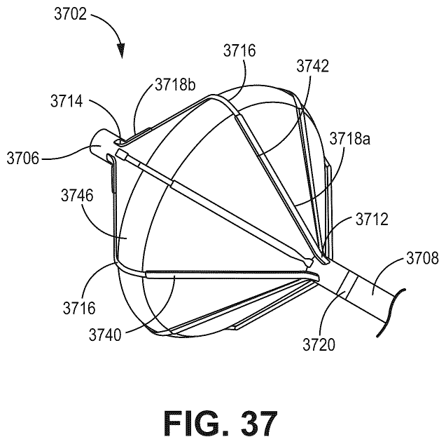

Referring now to FIG. 37, which illustrates a catheter 3700 that includes an end assembly 3702 coupled to a shaft 3708. The shaft 3708 may include at least one shaft electrode 3720. The shaft electrode 3720 may be a ring electrode. As shown in FIG. 37, the catheter 3700 includes one shaft ring electrode 3720 positioned along an outer surface of the shaft 3708 proximally adjacent to the end assembly 3702. However, a plurality of shaft electrodes may be positioned along the shaft 3708.

Like the end assemblies illustrated in FIGS. 3A and 3C, the end assembly 3702 illustrated in FIG. 37 includes a basket 3740 and an expandable balloon 3746 located within the space defined by the basket 3740. As discussed above, the basket 3740 includes a plurality of splines 3742 with proximal ends 3712 and distal ends 3714. A proximal end of a spline may be attached to the outer shaft and a distal end of the spline is attached to another shaft, such as an inner shaft, as illustrated in FIG. 3A, or a basket support shaft as illustrated in FIGS. 3B-3C. The distal end of the basket 3740 may be coupled to a distal coupler, such as the distal couplers shown and described above for FIGS. 4-9; the proximal end of the basket 3740 is coupled to a proximal coupler, such as the proximal couplers shown and described above for FIGS. 10-20; the basket 3740 includes a tether, such as tethers shown and described above for FIGS. 34-36; and/or one or more of the catheter shafts 3706, 3708 includes a cut shaft pattern, such as the patterns 2104, 2210, 2810 shown and described above for FIGS. 21-33.

The ends of the balloon 3746 may be coupled to the shaft 3708, or to the proximal and distal couplers, as discussed above. An expandable balloon 3746, positioned within the space defined by the basket 3740, extends across the length of the splines. The balloon 3746 extends between the distal end of the outer shaft and the distal end of the inner shaft in some embodiments. As discussed above, the balloon 3746 may be selectively expanded via fluid or other media provided by the fluid source (shown in FIG. 1). The balloon 3746 may have a plurality of expansion/expansion states (e.g., uninflated/unexpanded, inflated/expanded, overinflated/over-expanded). When the balloon 3746 is in an unexpanded state, the outer surface of the balloon 3746 may be a distance away from (does not contact) the inner surface of the basket 3740. The balloon 3746 may be in an unexpanded state before expansion media is supplied to the balloon 3746 and after the expansion media is withdrawn from the balloon 3746. The outer surface of the balloon 3746 in an expanded state may contact the inner surface of at least one of the plurality of splines without extending between the sides of adjacent splines, as illustrated for example in FIG. 37. The cross-sectional view of FIG. 3A also illustrates a balloon in an expanded state. Contact between the balloon 3746 and the basket may provide mechanical support to the end assembly 3702. Mechanical support provided by the balloon in the expanded state may maintain the basket-balloon end assembly 3702 in an expanded state. In embodiments where the balloon is comprised of an insulative material and electrodes 3716 are provided on the splines of the basket, the balloon in an expanded state may reduce energy loss, which may increase the therapeutic efficacy of an ablative force delivered by an electrode 3716 located on the plurality of splines 3742. In other words, more of the ablative energy may be directed towards the target tissue. A reduction in energy loss may be due to the position of the balloon relative to the electrodes and adjacent tissue. The efficacy of the ablative force may increase because larger sized lesions are generated.

The inner shaft 3708 may be slidably positioned within the outer shaft 3706. In this way, the basket 3740 may be operable between a collapsed state and an expanded state. When the inner shaft 3708 slides distally relative to the outer shaft 3706, the basket 3740 may be axially compressed into a collapsed state. The basket 3740 may be in the collapsed state during navigation of the catheter to a target site within a body lumen. When the inner shaft slides 3708 proximally relative to the outer shaft 3706, the basket 3740 may be radially expanded into an expanded state. The basket 3740 may be selectively expanded to optimize surface contact with target tissue. Basket 240 and/or basket 340 discussed above may also be expanded and contracted in this way.

Providing an excess of expansion media to the balloon may cause the balloon to be in an over-expanded state. In an over-expanded state, the outer surface of the balloon may contact the inner surface of at least one of the plurality of splines 3742 and/or extend between one or more pairs of adjacent splines 3742. The balloon 3746 in an over-expanded state may extend radially beyond the outer surface of the basket 3740.

The end assembly 3702 may further include at least one electrode 3716 configured for mapping and/or ablation. The at least one electrode 3716 may be located on and/or coupled to the basket 3740 and/or the balloon 3746. For example, like basket 240 and basket 340, basket 3740 has one or more electrodes 3716. Although, in the embodiment illustrated in FIG. 37, each spline 3742 includes one electrode 3716, a spline may have no electrodes or a plurality of electrodes. The electrodes may be mounted or otherwise affixed to an outer surface of at least one spline. The electrodes may be exposed portions of a spline formed of a conductive material. The exposed portion may be along the exterior surface of the spline and the interior surface of the spline is insulated. The electrode may be positioned along a middle region of the spline (e.g., at or near the portion of the spline 3742 extending furthest from a longitudinal centerline of the catheter when the basket is expanded). A proximal end of the electrode may be positioned adjacent to the middle of the spline and the distal end of the electrode is positioned closer to the distal end of the spline than to the middle of the spline. For example, as illustrated in FIG. 37, each spline 3742 includes a single electrode 3716, an exposed portion, with a proximal end located adjacent to the middle of the spline 3742 (e.g., at or near the portion of the spline 3742 extending furthest from a longitudinal centerline of the catheter when the basket is expanded) and a distal end located closer to the distal end 3714 of the spline than to the middle of the spline. Insulated portions 3718a, 3718b are positioned on either side of the exposed portion, electrode 3716. As discussed above, the electrode may receive an electrical signal from the mapping and/or ablation system 118 via cable 116 and one or more wires included within the outer shaft 3706.

Nitinol is one non-limiting example of a conductive material that may be utilized for spline. Insulating material may be provided on portion(s) of the spline that are not intended to function as an electrode. An insulative cover on a spline may be selectively removed to expose sections of the conductive spline. The insulating material may be one or more of: a heat-shrink material (e.g., PET), a polymer tubing, or a polymer coat. A polymer coat may be applied to a spline by spraying or dip coating. Exemplary polymeric materials that may be utilized for the insulating material include PET, polyimide, and/or polyether block amide (PEBA).

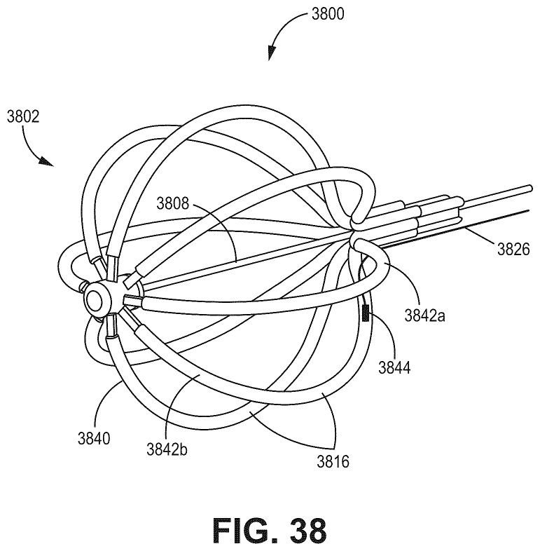

The catheter may further include at least one sensor configured to measure a characteristic indicative of the inflation/expansion state of the balloon (shown in FIG. 38). The sensor is a conductive sensor configured to measure an impedance between the sensor (a first electrode) and a second electrode that is indicative of the expansion state of the balloon. For example, in some embodiments, the ECU 124 of computer system 122 executes instructions to create an electrical circuit between the sensor (the first electrode) and the second electrode. The conductive sensor may be an electrode, or an exposed end of a conducting wire. The second electrode may be an electrode coupled to the basket, an electrode coupled to the balloon, and/or an electrode coupled to a catheter shaft, e.g., the inner shaft and/or the outer shaft. The sensor may be a pressure sensor configured to measure pressure. For example, the sensor may measure a pressure between the sensor and the balloon that is indicative of the expansion state of the balloon. The pressure sensor may be a mechanical pressure sensor or an electrical pressure sensor. A mechanical measurement may be converted into an electrical signal. For example, a microelectromechanical system (MEMS) sensor may convert mechanical pressure into an electrical signal.

The sensor may be coupled to the end assembly and/or the distal end of the outer shaft. For example, FIG. 38 illustrates an embodiment of a catheter 3800 with a sensor 3844 coupled to the basket 3840 of an end assembly 3802 (the balloon is not shown in this view). Like basket 3740, basket 3840 includes a plurality of splines 3842 and at least one electrode 3816 (two electrode regions identified). Basket 3840 also includes a support shaft 3808, as illustrated in FIG. 3B. The sensor 3844 is coupled to a wire 3826 extending through the outer shaft (not shown in this view). The wire 3826 may be configured to carry signals and/or communicate information about the measured characteristic to the computer system 122.

Embodiments with a plurality of sensors may have one or more sensors located in a proximal region of the end assembly (proximal to the midpoint of the end assembly), one or more sensors located in a distal region of the end assembly (distal to the midpoint of the end assembly), or one or more sensors located in both the proximal and distal regions of the end assembly. For example, sensor 3844 illustrated in FIG. 38, is coupled to a proximal region of the basket. Although, the sensor 3844 illustrated in FIG. 38 is coupled to a spline 3842b with a single electrode 3816, the sensor may be coupled to a spline that lacks an electrode or has a plurality of electrodes. The sensor 3844 may be coupled to an inner surface of a spline 3842b, as illustrated in FIG. 38.

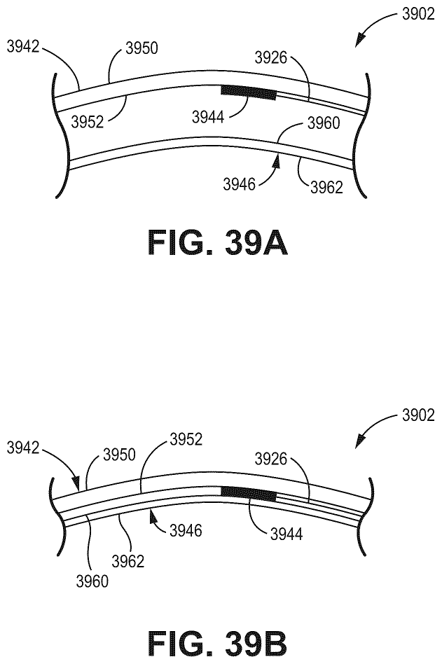

FIGS. 39-41 are views illustrating features of a sensor coupled to a basket-balloon end assembly. In FIGS. 39A, 40A, and 41A, the balloon is in an unexpanded state while in FIGS. 39B, 40B, and 41B, the balloon is in an expanded state. As discussed above, the sensor may be coupled to a spline that lacks an electrode. For example, as illustrated in FIG. 39A, the end assembly 3902 includes a sensor 3944 coupled to the inner surface 3952 of a spline 3942 that lacks an electrode. The sensor 3944 is coupled to a wire 3926 configured to carry signals and/or communicate information about the measured characteristic to the computer system 122. As discussed above, the balloon 3946 may be made of an insulating material. The spline 3942 has an outer surface 3950 and an inner surface 3952 and the balloon 3946 has an outer surface 3960 and an inner surface 3962. When the balloon 3946 is in an unexpanded state, the outer balloon surface 3960 does not contact the inner spline surface 3952 and/or the sensor 3944, as illustrated in FIG. 39A. When the balloon 3946 is in an expanded state, the outer balloon surface 3960 contacts the inner spline surface 3952 and/or the sensor 3944, as illustrated for example in FIG. 39B.

As discussed above, the sensor 3944 may be a pressure sensor and the pressure is the measured characteristic indicative of balloon expansion state. The measured pressure may indicate contact, as illustrated in FIG. 39B, or no-contact, as illustrated in FIG. 39A. This relationship may be utilized to differentiate between a balloon in an unexpanded state (no-contact) and a balloon in an expanded state (contact). The sensor may measure an amount of pressure and/or a change in pressure. As the balloon expands/contracts the measured pressure may increase/decrease. Continued expansion of the balloon after initially contacting the sensor 3944 may cause the measured pressure to increase. In this way, an uninflated state may be distinguished from either an inflated state (correlated to a first pressure) or an over-expanded state (correlated to a second pressure greater than the first pressure). An evaluation of a change in pressure may be utilized to determine if the balloon is expanding or deflating.

As discussed above, impedance, measured between two electrodes/conductors, may be the characteristic indicative of balloon expansion state measured by the sensor. The sensor 3944 may be a conductor (a first electrode). For example, the sensor may be an electrode or an exposed wire. The second electrode may be an electrode on another spline, an electrode coupled to an outer surface of the balloon, or a shaft electrode, such as electrode 3720. The ECU 124 of computer system 122 may execute instructions to create an electrical circuit between the sensor (first electrode) and the second electrode. A lower impedance may be measured when the balloon is in an unexpanded state than when the balloon is in an expanded state. When catheter is used in a body lumen containing blood, a lower impedance may be measured because blood contains electrolytes which ionize to conduct electricity. Expansion of the balloon may cause the balloon to contact and/or cover the sensor (first electrode), which may increase the measured impedance. The change in measured impedance between an unexpanded state and an expanded state may be greater when the balloon is formed of insulating material.

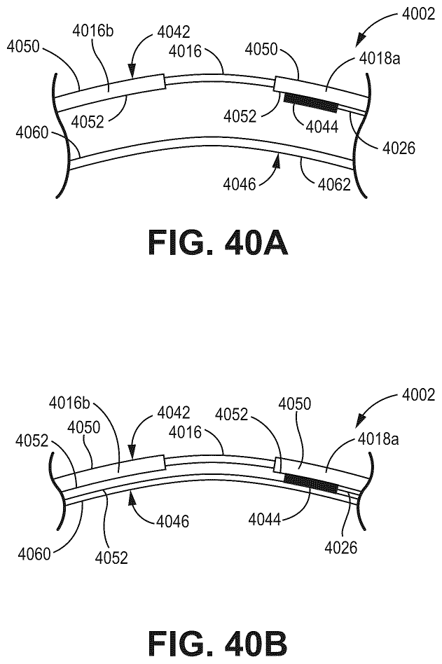

FIGS. 40A-40B illustrate an embodiment of an end assembly 4002 that includes a sensor 4044 coupled to a spline 4042 with an electrode 4016. The sensor 4044 is coupled to the inner surface 4052 of the spline 4042 that lacks an electrode. A sensor 4044 coupled to wire 4026. Like wire 3826, wire 4026 may be configured to carry signals and/or communicate information about the measured characteristic to the computer system 122. The spline 4042 includes insulative regions 4018a, 4018b and an outer surface 4050. The balloon 4046 has an outer surface 4060 and an inner surface 4062. As discussed above, the balloon 4046 may be made of an insulating material. When the balloon 4046 is in an unexpanded state, the outer balloon surface 4060 does not contact the inner spline surface 4052 and/or the sensor 4044, as illustrated in FIG. 40A. When the balloon 4046 is in an expanded state, the outer balloon surface 4060 contacts the inner spline surface 4052 and/or the sensor 4044, as illustrated for example in FIG. 40B.

As discussed above, the sensor 4044 may be a pressure sensor and the measured pressure is the characteristic indicative of balloon expansion state. The measured pressure may indicate contact, as illustrated in FIG. 40B, or no-contact, as illustrated in FIG. 40A. This relationship may be utilized to differentiate between a balloon in an unexpanded state (no-contact) and a balloon in an expanded state (contact). The sensor may measure an amount of pressure and/or a change in pressure. As the balloon expands/contracts the measured pressure may increase/decrease. Continued expansion of the balloon after initially contacting the sensor 4044 may cause the measured pressure to increase. In this way, an uninflated state may be distinguished from either an inflated state (correlated to a first pressure) or an over-expanded state (correlated to a second pressure greater than the first pressure). An evaluation of a change in pressure may be utilized to determine if the balloon is expanding or deflating.

As discussed above, impedance, measured between two electrodes/conductors, may be the characteristic indicative of balloon expansion state measured by the sensor. In these embodiments, the sensor 4044 is a conductor (a first electrode). For example, the sensor may be an electrode or an exposed wire. As illustrated in FIGS. 40A-B, the second electrode is the spline electrode 4016. However, the second electrode may be an electrode on another spline, an electrode coupled to an outer surface of the balloon, or a shaft electrode, such as electrode 3720. The ECU 124 of computer system 122 may execute instructions to create an electrical circuit between the sensor (first electrode) and the second electrode. A lower impedance may be measured when the balloon is in an unexpanded state than when the balloon is in an expanded state. When catheter is used in a body lumen containing blood, a lower impedance may be measured because blood is a conductor. Expansion of the balloon may cause the balloon to contact and/or cover the sensor (first electrode), which may increase the measured impedance. The change in measured impedance between an unexpanded state and an expanded state is greater when the balloon is formed of insulating material.

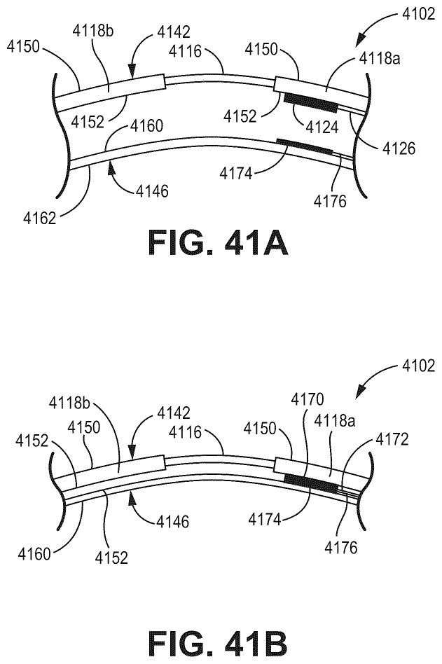

FIGS. 41A-B illustrates features of an end assembly 4102 that includes a sensor 4144 coupled to a spline 4142. Although, as illustrated, the sensor 4144 is coupled to the inner surface 4152 of the spline 4142 with an electrode 4116, the sensor may be coupled to the inner surface of a spline that lacks an electrode. The sensor 4144 is coupled to wire 4126. Like wire 4126, wire 4126 may be configured to carry signals and/or communicate information about the measured characteristic to the computer system 122. The spline 4142 includes insulative regions 4118a, 4118b and an outer surface 4150. The balloon 4146 includes an outer surface 4160, an inner surface 4162, and a plate 4174 configured to contact sensor 4144 when the balloon is in an expanded state, as illustrated in FIG. 41B. As illustrated, the plate 4174, coupled to the outer surface 4160, is an electrode coupled to wire 4176. The plate 4174 may be a piece of material that is stiffer and/or harder than the balloon material. For example, a section of the polymer forming the balloon wall may be cross-linked. As discussed above, the balloon 4146 may be made of an insulating material. When the balloon 4146 is in an unexpanded state, the outer balloon surface 4160 does not contact the inner spline surface 4152 and/or the sensor 4144, as illustrated in FIG. 41A. When the balloon 4146 is in an expanded state, the outer balloon surface 4160 contacts the inner spline surface 4152 and/or the sensor 4144, as illustrated for example in FIG. 41B.

As discussed above, the sensor 4144 may be a pressure sensor and the measured pressure is the characteristic indicative of balloon expansion state. The measured pressure may indicate contact with plate 4174, as illustrated in FIG. 41B, or non-contact with plate 4174, as illustrated in FIG. 41A. This relationship may be utilized to differentiate between a balloon in an unexpanded state (non-contact) and a balloon in an expanded state (contact). The sensor may measure an amount of pressure and/or a change in pressure. As the balloon expands/contracts the measured pressure may increase/decrease. Continued expansion of the balloon after initially contacting the sensor 4144 may cause the measured pressure to increase. In this way, an uninflated state may be distinguished from either an inflated state (correlated to a first pressure) or an over-expanded state (correlated to a second pressure greater than the first pressure). An evaluation of a change in pressure may be utilized to determine if the balloon is expanding or deflating.

As discussed above, impedance, measured between two electrodes/conductors, may be the characteristic indicative of balloon expansion state measured by the sensor. The sensor 4144 may be a conductor (a first electrode). For example, the sensor may be an electrode or an exposed wire. The second electrode may be the plate 4174 (balloon electrode), the spline electrode 4116, an electrode on another spline, or a shaft electrode, such as electrode 3720. The ECU 124 of computer system 122 may execute instructions to create an electrical circuit between the sensor (first electrode) and the second electrode. The measured impedance may be high when the balloon is in the expanded state and low when the balloon is in the unexpanded state. For example, as discussed above with reference to FIGS. 39-40, when the balloon is in an expanded or over-expanded state (in contact with the sensor) the impedance between the sensor and a second electrode is greater than when the balloon is in an unexpanded state (not in contact with the sensor). The measured impedance may be low when the balloon is in an expanded state (in contact with the sensor) and high impedance when the balloon is in an unexpanded state (not in contact with the sensor). For example, when the balloon 4146 is expanded so that the balloon electrode 4174 contacts the sensor 4144 (first electrode) (as shown in FIG. 41B), a short circuit is formed between the sensor 4144 and the balloon electrode 4174, which results in lower measured impedance compared to impedance measured when the balloon electrode 4174 and the sensor 4144 are separated (as shown in FIG. 41A).

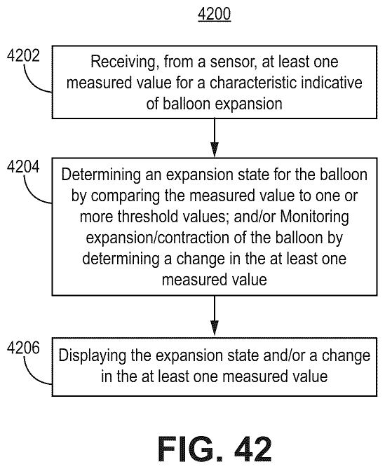

As discussed above, the expansion state of the balloon may affect performance of the end assembly. The characteristic measured by the sensor may be utilized to determine an expansion state of a balloon. FIG. 42 illustrates a flowchart of a method 4200 to determine the expansion state of a balloon. The computer system 122 may be configured to execute method 4200. The expansion state determined by method 4200 may be utilized to modify the expansion state of the balloon, for example by adding/removing expansion media from the balloon.

Turning to the flowchart of FIG. 42, at step 4202, method 4200 includes receiving, from a sensor, at least one measured value for a characteristic indicative of balloon expansion. The computer system 122 may receive the measured value from the sensor. As discussed above, the sensor may be a pressure sensor configured to measure a pressure between the sensor and the balloon. The measured pressure may be the pressure of the outer surface of the balloon against the sensor when the balloon is in an expanded state, as discussed above with reference to FIGS. 39B and 40B. The polymeric material, forming the portion of the balloon configured to press against the sensor, is cross-linked. The measured pressure may be the pressure between a plate coupled to the outer surface of the balloon against the sensor when the balloon is in an expanded state, as discussed above with reference to FIGS. 41B. The sensor may be conductive and configured to measure an impedance between a sensor and a second electrode. The sensor (first electrode) and the second electrode may be coupled to different splines, as discussed above with reference to FIG. 39A. The sensor and the second electrode may be coupled to the same spline, as illustrated for example in FIG. 40A. The sensor may be coupled to a spline and the second electrode is coupled to the balloon, as discussed above with reference to FIG. 41A. The sensor may be coupled to the end assembly (basket or balloon) and the second electrode is coupled to the shaft, such as shaft electrode 3720.

At step 4204, method 4200 includes determining an expansion state for the balloon by comparing the measured value to one or more threshold values; and/or monitoring expansion/contraction of the balloon by determining a change in measured values. The one or more threshold values may include a first threshold value selected to differentiate between an unexpanded state and an expanded state (e.g., a first threshold value). For example, a measured pressure value less than the threshold pressure value indicates that the balloon is in an unexpanded state (no-contact) while a measured pressure value greater than the threshold pressure value indicates that the balloon is in an expanded state (contact). The one or more threshold values may further include a second threshold value greater than the first threshold value to differentiate between an expanded state and an over-expanded state. For example, a measured value greater than the first threshold value and the second threshold value indicates that the balloon is in an over-expanded state while a measure value greater than the first threshold value and less than the second threshold indicates that the balloon is in an expanded state. Table 2 summarizes determining the expansion state of a balloon utilizing one or more threshold values.

TABLE 2

| | Balloon in | Balloon in Expanded | Balloon in Over- |
|---|---|---|---|
| | Unexpanded State | State | expanded State |
| Measured | Measured value < | First threshold value ≤ Measured value | |
| Characteristic - | First threshold | | |
| Pressure | value | | |
| Measured | Measured value < | First threshold value ≤ | First threshold value < |
| Characteristic - | First threshold | Measured value < | Second threshold |
| Pressure | value | Second threshold | value ≤ Measured |
| | | value | value |
| Measured | Measured value < | First threshold value ≤ | First threshold value < |
| Characteristic - | First threshold | Measured value < | Second threshold |
| Impedance | value | Second threshold | value ≤ Measured |
| | | value | value |
| Measured | First threshold | Measured value ≤ First threshold value | |
| Characteristic - | value < Measured | | |
| Impedance | value | | |
| (FIG. 41A-B) | | | |

As can be seen in Table 2, some measured characteristics only differentiate between an unexpanded state and an expanded state while other measured characteristics can differentiate between an unexpanded state, an expanded state, and an over-expanded state.

A change in the measured characteristic may be utilized to monitor expansion/contraction of the balloon. For example, a positive change in the measured characteristic may indicate that the balloon is expanding while a negative change in the measured characteristic indicates that the balloon is contracting. Continued expansion of the balloon may result in the value of the measured characteristic to increase. A negative change in the measured characteristic may indicate that the balloon is expanding while a positive change in the measured characteristic indicates that the balloon is contracting, as discussed for FIGS. 41A-B.

At step 4206, method 4200 includes displaying the expansion state and/or a change in the at least one measured value. The expansion state may be displayed on a monitor of the computer system 122. Based on the displayed expansion state, the user may either maintain the amount of expansion media in the balloon or adjust the expansion of the balloon by adding or removing expansion media. The displayed change in the at least one measured value may be utilized to monitor expansion of the balloon. For example, monitoring a change in the measured characteristic may be utilized to maintain the expansion state of the balloon.

A medical device may include at least one shaft and a basket. The distal end of the basket may be coupled to a distal coupler, such as one of the distal couplers shown and described above for FIGS. 4-9 and 48-52. The proximal end of the basket may be coupled to a proximal coupler, such as one of the proximal couplers shown and described above for FIGS. 10-20 and 53-62. The basket may further include a plurality of tethers, such as tethers shown and described above for FIGS. 34-36. The medical device may further include a balloon positioned inside the basket, as shown and described above for FIGS. 3A, 3C, 49-50, and 62. The balloon may be coupled to the distal coupler and to the proximal coupler. At least one end of the balloon may be inverted. The medical device may further include at least one sensor configured to measure a characteristic indicative of the inflation/expansion state of the balloon, as discussed above for FIGS. 37-41. The at least one shaft may include a basket support shaft. The basket support shaft may be secured to the proximal coupler and free floating in the distal coupler. The at least one shaft may further include a cut shaft pattern, such as the patterns 2104, 2210, 2810 shown and described above for FIGS. 21-33.

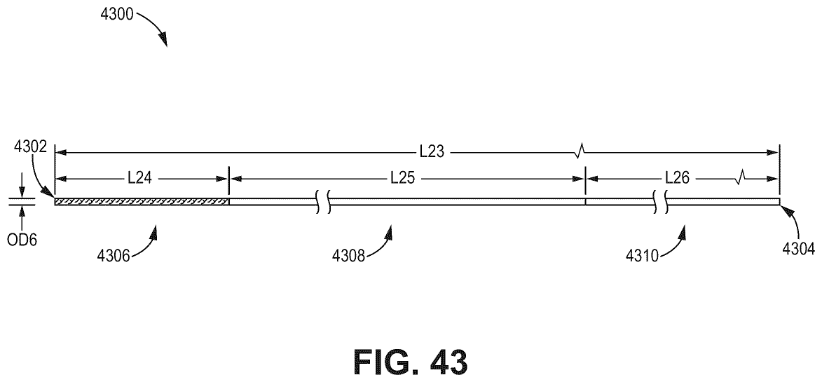

FIG. 43 illustrates features of a shaft 4300. The shaft 4300 may be utilized in one or more medical devices described above. For example, shaft 4300 may be utilized as an inner shaft of a medical device comprising an expandable component, such as catheter 202 shown in FIG. 2 or catheter 302 shown in FIG. 3, e.g., utilized for the inner shaft 308. As discussed below in greater detail, the shaft 4300 may include one or more features that improve performance compared to a typical shaft. For example, the shaft 4300 includes features to reduce kinking of the central lumen. In some applications, the features to reduce kinking are in a section of the shaft 4300 configured to be positioned within an expandable component and the shaft 4300 does not require additional components, such as support tubes, to reduce kinking of the central lumen, while maintaining the trackability of the shaft 4300 over a guidewire positioned in a central lumen. As another example, the outer diameter of the shaft 4300 is smaller than typical support tubes extending through an expandable component.

A reduced outer diameter of the shaft 4300 may facilitate insertion and movement through a curved sheath/shaft. Further, the shaft 4300 described herein, when incorporated into a catheter with a basket, provides a “softer” basket relative to conventional catheters with a basket because the shaft 4300 has a reduced buckle force. In one aspect, the reduced buckle force may provide easier insertion and/or advancement of the shaft 4300 through a curved sheath/shaft. Additionally, the structure of the shaft 4300 reduces or prevents formation of a leak path through the shaft wall, e.g., from a central lumen to an outer surface of the shaft 4300.

Returning to FIG. 43, the shaft 4300 has an outer diameter OD6 and a length L23 measured from a first end 4302 to a second end 4304. The outer diameter OD6 may be substantially constant. The outer diameter OD6 may be approximately 0.061″ to approximately 0.064″. The shaft 4300 may include at least two sections. As illustrated in FIG. 43, the shaft 4300 has a first section 4306 extending from the first end 4302 for a length L24, a second section 4308 extending from the first section 4306 for a length L25, and a third section 4310 extending from the second section 4308 to the second end 4304 for a length L26. The second section 4308 may have the greatest length L25. The length L24 of first section 4306 may be less than the length L25 of second section 4308 and/or the length L26 of the third section 4310. As shown in FIG. 43, the length L24 of the first section 4306 is less than the length L26 of the third section 4310 which is less than the length L25 of the second section 4308 (L24<L26<L25). The length L24 of the first section 4306 may be about 2-4% of the length L23 of the shaft 4300, the length L25 of the second section 4308 may be about 74-78% of the length L23 of the shaft 4300, and the length L26 of the third section 4310 may be about 19-23% of the length L23 of the shaft 4300. As discussed below in greater detail, the first section 4306 of the shaft 4300 may be positioned within an expandable end assembly, such as a basket and/or balloon, and the first section 4306 is configured to prevent the center lumen from kinking. Thus, the length L24 of the first section 4306 of the shaft 4300 may be sized so that the first section 4306 extends across the lumen of the expandable component.

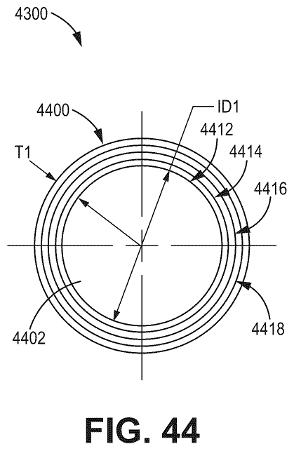

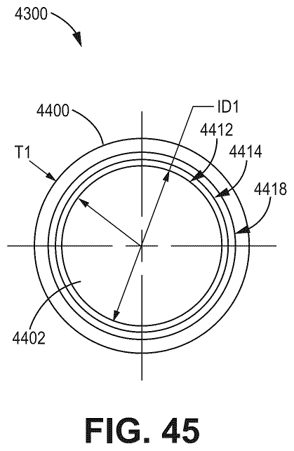

As shown in FIGS. 44 and 45, the wall 4400 of the shaft 4300 includes a plurality of layers (4412-4418), has a thickness (e.g., T1), and defines a central lumen 4402 with a diameter ID. The plurality of layers includes an inner layer 4412, an outer layer 4418, and one or more intermediate layers 4414, 4416. The first section 4306 of the shaft 4300 may include two intermediate layers 4414, 4416, as shown in FIG. 44, while the second and third sections 4308, 4310 include a single intermediate layer 4414, as shown in FIG. 45. The inner layer 4412 and the first intermediate layer 4414 may extend the length of the shaft 4300. The plurality of layers 4412-4418 may include at least one polymeric layer and at least one metallic layer. The inner layer 4412 may be a polymeric layer, the intermediate layers 4414, 4416 may be metallic layers, and the outer layer 4418 may be a polymeric layer. The metallic layer may be a reinforcement layer. For example, the metallic reinforcement layer may be a braid or a coil. The first section 4306 may include a first intermediate layer 4414 that is a metallic braid and a second intermediate layer 4416 that is a metallic coil. As noted above, the first intermediate layer 4414 may extend the length of the shaft 4300 while the second intermediate layer 4416 extends for the length of the first section 4306. In one aspect, a braided first intermediate layer 4414 that extends the length of the shaft 4300 may provide resistance to crushing or kinking along the length of the entire shaft and the coiled second intermediate layer 4416 provides enhanced pushability and kink resistance, compared to the braid alone, in the first section 4306 of the shaft 4300.

A flat wire may be utilized for at least one of the metallic reinforcement layers. In one aspect, utilizing flat wires provide desired mechanical properties in the critical axis strength and pushability along the long axis of the shaft-without increasing wall thickness to the degree a round wire would. The wires forming the braid may be smaller than the wire forming the coil. The dimensions of the wires utilized for the braided reinforcement layer may be 0.001″×0.003″ and the dimensions of the wire utilized for the coil may be 0.002″×0.006″. Wires having other wire dimensions may be utilized for the braided reinforcement layer and/or the coil reinforcement layer.

Some non-limiting examples of polymers and metals that may be utilized for the shaft 4300 include thermoplastic polymers, elastomeric polymers, fluoropolymers, polyether block amides (PEBA), polytetrafluoroethylene (PTFE), stainless steel, and nitinol. The shaft 4300 may include one or more areas, bands, coatings, members, etc. that is (arc) detectable by imaging modalities such as X-Ray, MRI, ultrasound, etc.

Different materials may be utilized for the outer layer 4418. Polymers with different durometer hardnesses may be utilized for the outer layer 4418 of one or more of the sections 4306, 4308, 4310 of the shaft 4300. For example, a higher durometer hardness polymer may be utilized for the outer layer 4418 of the second section 4308 and lower durometer hardness polymers utilized for the outer layers 4418 of the first and second sections 4306, 4310. In one example, a first polymer with a first durometer hardness is utilized for the outer layer 4418 of the first section 4306, a second polymer with a second durometer hardness is utilized for the outer layer 4418 of the second section 4308, and a third polymer with a third durometer hardness is utilized for the outer layer 4418 of the third section 4310, where the second durometer hardness is less than the first durometer hardness which is less than the third durometer hardness. For example, the first durometer hardness may be 55 D, the second durometer hardness may be 35 D, and the third durometer hardness may be 72 D.

The durometer hardness of the outer layer 4418, type of reinforcement layer, and/or number of reinforcement layers may affect flexibility. Providing a more flexible section between two stiffer sections may improve the performance of the shaft 4300. For example, a stiffer first section 4306 may provide support to a basket, reduce/prevent kinking of the central lumen 4402 in the first section 4306, and/or reduce/prevent formation of a leak path, as discussed above. As another example, a more flexible second section 4308 may improve trackability of the shaft 4300. The flexible second section 4308 may also enable the shaft 4300 to stretch. In one aspect, this property may allow the basket to expand (lengthen) and retract (shorten) when the shaft 4300 is utilized to support a basket of a medical device. Additionally, a stiffer third section 4310 extending from a handle may improve the pushability of the shaft 4300.

The diameter ID of the central lumen 4402 may range from approximately 0.035″ to approximately 0.050″. The diameter ID of the central lumen 4402 may be configured to receive a guidewire. The diameter ID of the central lumen 4402 may be configured to transmit a fluid to the first end 4302 at the same time a guidewire is positioned in the central lumen 4402. The central lumen 4402 may have a substantially constant diameter ID. As a non-limiting example, the diameter ID of the central lumen 4402 may be approximately 0.044″. The diameter ID of the central lumen 4402 may vary. For example, the diameter ID of the central lumen 4402 may be tapered. The central lumen 4402 extending through the first section 4306 may have a first diameter, e.g., ID1, while the central lumen 4402 extending through the second and third sections 4308, 4310 may have a second diameter, e.g., ID2, that is less than the first diameter ID1. For example, the central lumen 4402 extending through the first section 4306 has a first diameter ID1 of approximately 0.044″ and the central lumen 4402 extending through the second and third sections 4308, 4310 has a second diameter ID2 of approximately 0.05″.

In one aspect, by defining a smaller diameter ID central lumen 4402, the wall 4400 has a thickness T1 that is sufficient to accommodate two intermediate reinforcement layers while optimizing the cross-sectional area of the central lumen 4402 for fluid flow when a guide wire is positioned in the central lumen 4402. The wall thickness T1 may be substantially constant along the length of the shaft 4300. For example, as shown in FIGS. 44 and 45, the outer layer 4418 in the second and third sections 4308, 4310 may be thicker than the outer layer 4418 in the first section 4306 to provide the shaft 4300 with a substantially constant wall thickness T1. As discussed above, the outer diameter OD6 of the shaft 4300 is smaller than typical support tubes that may extend through the lumen of an expandable component. In one aspect, a decreased wall thickness T1 may provide the shaft 4300 with a smaller outer diameter OD6. The wall thickness T1 may be approximately 0.008″ to approximately 011″. In contrast, the braided shaft in addition to a typical support tube has a wall thickness of 0.023″.

One exemplary embodiment of a shaft 4300 as described above defines a central lumen 4402 with a diameter ID and includes a distal first section 4306 extending from the distal first end 4302, a second section 4308 extending from the first section 4306, a third section 4310 extending from the second section 4308, an inner layer 4412 of PTFE extending from the distal first end 4302 to the second end 4304, a stainless steel braid over the PTFE inner layer 4412 and extending from the first end 4302 to the second end 4304, where the first section 4306 further includes a stainless steel coil over the stainless steel braid, and a 55 D PEBA outer layer 4418 over the stainless steel coil, the second section 4308 further includes a 35 D PEBA outer layer 4418 over the stainless steel braid, and the third section 4310 further includes a 72 D PEBA outer layer 4418 over the stainless steel braid. The PEBA outer layers 4418 may be reflowed over the stainless steel coil and/or the stainless steel braid. The wire forming the stainless steel braid may be 0.001″×0.003″ wire and the wire forming the stainless steel coil may be 0.002″×0.006″ wire. The stainless steel coil may have a pitch of 0.015″. The diameter ID of the central lumen 4402 may range from approximately 0.035″ to approximately 0.050″.

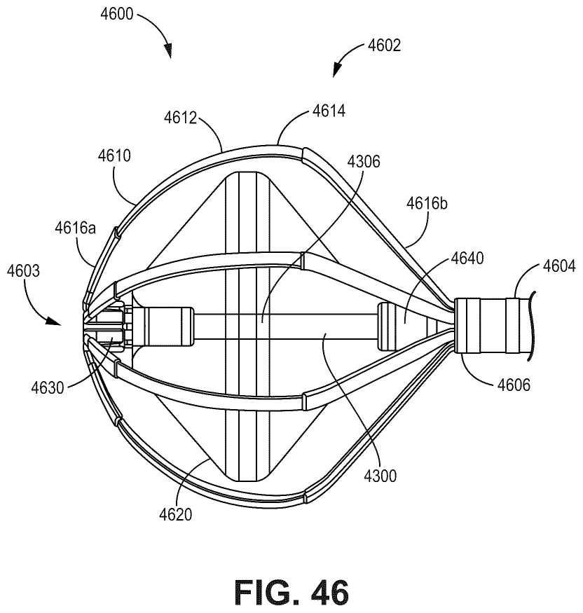

FIG. 46 illustrates a medical device 4600 with the shaft 4300 extending through a coaxial outer shaft 4604 and through the lumen of an expandable end assembly 4602. As discussed above, the shaft 4300 and the outer shaft 4604 may slide relative to one another. For example, the medical device 4600 may include a handle (not shown) configured to slide the shaft 4300 and the outer shaft 4604 relative to one another. The expandable end assembly 4602 shown in FIG. 46 includes a balloon 4620 positioned inside a basket 4610. The expandable end assembly 4602 may have a blunt and/or atraumatic distal end 4603. At least one end of the balloon 4620 may be inverted. The basket 4610 may include a plurality of splines 4614 coupled to the distal and proximal couplers 4630, 4640. Each spline 4614 may include at least one electrode 4612 and a plurality of insulative regions (collectively 4616, insulative regions 4616a and 4616b are identified). As discussed above, in some embodiments, the basket 4610 is configured for ablation and/or mapping. The basket 4610 may further include a plurality of tethers, such as tethers shown and described above for FIGS. 34-36.

As shown in FIG. 46, the basket 4610 and the balloon 4620 are coupled to the distal and proximal couplers 4630, 4640. The expandable end assembly 4602 may be coupled to the shaft 4300 by a distal coupler 4630 and the outer shaft 4604 by a proximal coupler 4640. As discussed above, the proximal coupler 4640 may include a retention ring 4606. Relative movement of the shaft 4300 and the outer shaft 4604 may be utilized to expand/contract the expandable end assembly 4602. As shown in FIG. 46, the distal coupler 4630 may be configured to receive the first end 4302 of the shaft 4300 and the proximal coupler 4640 may be configured to surround the shaft 4300. The distal coupler 4630 may be a distal coupler as shown and described above for FIGS. 4-9 and 48-52. The proximal coupler 4640 may be a proximal coupler as shown and described above for FIGS. 10-20 and 53-62. As described above, the inflation media to inflate the balloon 4620 may be conveyed via the proximal coupler 4640 or via the shaft 4300.

The first section 4306 may span the lumen of the basket 4610 to provide support to the basket 4610. For example, the proximal end of the first section 4306 of the shaft 4300 may be coextensive with the proximal end of the basket 4610. The first section 4306 may extend beyond the proximal end of the basket lumen. For example, the first section 4306 may extend at most to a proximal end of the proximal coupler 4640. As discussed above, the first section 4306 includes a coiled reinforcement layer (e.g., second intermediate layer 4416) on a braided reinforcement layer (e.g., first intermediate layer 4414) to prevent kinking of the central lumen of the shaft section that spans the basket 4610.

The length of the distal first section 4306 may be about 2-4% of the length of the shaft 4300, the length of the second section 4308 may be about 74-78% of the of the shaft 4300, and the length of the third section 4310 may be about 19-23% of the length of the shaft 4300. The distal first section 4306 may be 1.6″±0.2″ long, the second section 4308 may be 42.9″±0.5″ long, and the third section 4310 may be 12.0″±0.5″ long, where the second section 4308 extends proximally from the distal first section 4306 and the third section 4310 extends proximally from the second section 4308. A handle of the medical device 4600 may be coupled to the third section 4310 (not shown). As discussed above, the handle may be configured to slide the shaft 4300 and the outer shaft 4604 relative to one another.

The medical device 4600 may further include at least one sensor configured to measure a characteristic indicative of the inflation/expansion state of the balloon 4620, as discussed above for FIGS. 37-41.

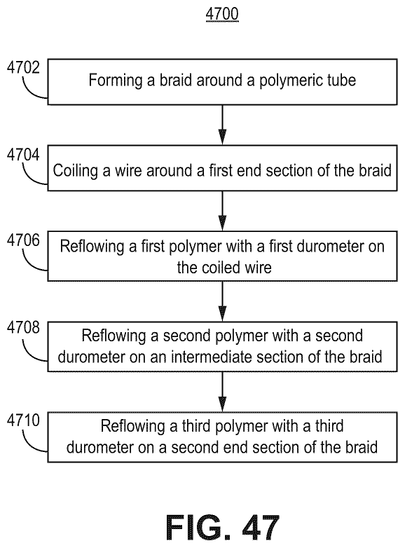

FIG. 47 is a flowchart of a method 4700 of forming a shaft 4300 defining a central lumen 4402 and including a first end 4302, a second end 4304, a first section 4306 with a first length L24 and extending from the first end 4302, a second section 4308 with a second length L25 and extending from the first section 4306, a third section 4310 with a third length L26 and extending from the second section 4308, as shown in FIGS. 43 and 44. The first length L24 may be less than the third length L26, which may be less than the second length L25. The central lumen 4402 may have a substantially constant diameter ID. The central lumen 4402 in the first section 4306 may have a smaller diameter ID than the diameter ID of the central lumen 4402 in the second and third sections 4308, 4310. The method 4700 may manufacture the shaft 4300 from the inside to the outside.

At step 4702, a braided layer is provided on an outer surface of a polymeric tube. The polymeric tube may form the inner layer 4412 and the braid may form a first intermediate layer 4414 of the shaft 4300. The polymeric tube may be formed on, or coupled to, a mandrel. The outer surface of the polymeric tube may be etched. As discussed below, etching the polymeric tube may enhance bonding to the polymeric tube. Providing a braided layer on the polymeric tube may comprise braiding wires around the polymeric tube. The wires may be braided in any pattern. The braid may be formed by flat and/or metallic wires. The braid may comprises 0.001″×0.003″ wires. The polymeric tube may comprise PTFE and the braid may comprise stainless steel wires.

At step 4704, a coiled layer is provided on the braided layer. The coiled layer may form a second intermediate layer 4416 in a first section 4306 of the shaft 4300. In one aspect, a braid is less susceptible to movement during the addition of the coil than the coil would be during addition of the braid. This may provide for an easier manufacturing process and/or a more uniform shaft 4300. Providing a coiled layer on the braided layer may include coiling a wire at a predetermined pitch around a first end section of the braided layer. The coiled layer may form a second intermediate layer 4416 of the first section 4306 of the shaft 4300. The combination of the coiled layer on the braided layer may improve performance of the shaft 4300. For example, the coiled wire and braid reinforcement may prevent the shaft 4300 from kinking. The first end section may be positioned at a distal end section of the shaft 4300. The wire forming the coil may be a metallic wire. The wire may comprise stainless steel. The wire may be a 0.002″×0.006″ wire coiled at a pitch of 0.015″.

At step 4706, a first polymer with a first durometer hardness is applied to the coiled layer. The first polymer may form the outer layer 4418 of the first section 4306 of the shaft 4300. The first polymer may be heated to reflow the first polymer into gaps or interstices between braid wires and/or adjacent windings of the coil wire. The reflowed first polymer may be bonded to the polymeric tube via the gaps or interstices. Additionally, the etched outer surface of the polymeric tube may enhance bonding of the first polymer to the polymeric tube. Filling gaps or interstices between braid wires and/or adjacent windings of the coil wire with the first polymer may minimize and/or eliminate formation of a leak path through the shaft wall from the central lumen 4402 if a kink forms in the first section 4306 of the shaft 4300.

At step 4708, a second polymer with a second durometer hardness is applied to an intermediate section of the braided layer. The second polymer may form the outer layer 4418 of the second section 4308 of the shaft 4300. The second polymer may be heated to reflow the second polymer into gaps or interstices between the braid wires. The reflowed second polymer may be bonded to the polymeric tube via the gaps or interstices. Additionally, the etched outer surface of the polymeric tube may enhance bonding of the second polymer to the polymeric tube. The second durometer hardness may be greater than the first durometer hardness.

At step 4710, a third polymer with a third durometer is applied to a second end section of the braided layer. The third polymer may form the outer layer 4418 of the third section 4310 of the shaft 4300. The third polymer may be heated to reflow the third polymer into gaps or interstices between the braid wires. The reflowed third polymer may be bonded to the polymeric tube via the gaps or interstices. Additionally, the etched outer surface of the polymeric tube may enhance bonding of the third polymer to the polymeric tube. The third durometer may be greater than the first durometer hardness and less than the second durometer.

What is claimed is:

1. A capless distal coupler for coupling a basket to a shaft of a catheter, the capless distal coupler comprising:
a first wall defining a central lumen;
a plurality of projections extending outward from the first wall; and
a plurality of slots, wherein each slot is defined by the first wall, and two of the plurality of projections, wherein each slot is configured to receive a single spline of a basket.

2. The capless distal coupler of claim 1, wherein each projection of the plurality of projections comprises a curved leading section with a greater radius of curvature than a curved trailing section.

3. The capless distal coupler of claim 1, wherein each slot is positioned opposite to another slot.

4. The capless distal coupler of claim 1, wherein the first wall defines at least one through hole for securing the capless distal coupler to the shaft.

5. The capless distal coupler of claim 1, wherein each projection defines a retention slot configured to receive a spline retention feature of a basket spline.

6. The capless distal coupler of claim 1, wherein the capless distal coupler is coupled to a shaft comprising:
a polymeric tube forming an inner layer of the shaft;
a braid positioned on, and coextensive with, the polymeric tube;
a coil positioned on a first end section of the braid; and
at least one polymer forming an outer layer of the shaft.

7. The capless distal coupler of claim 6, the at least one polymer comprising:
a first polymer with a first durometer hardness over the coil;
a second polymer with a second durometer hardness over an intermediate section of the braid; and
a third polymer with a third durometer hardness over a second end section of the braid.

8. The capless distal coupler of claim 1, further comprising at least one magnetic sensor positioned in a groove along an outer surface of the capless distal coupler.

9. The capless distal coupler of claim 1, wherein the plurality of projections and the plurality of slots are located in a basket securement region of the capless distal coupler, the capless distal coupler further comprising a balloon securement region proximal to the basket securement region.

10. The capless distal coupler of claim 9, wherein the balloon securement region comprises an overmolded section for securing a balloon to the capless distal coupler.

11. A medical device comprising:
a distal coupler comprising:
a basket securement region;
a balloon securement region proximal to the basket securement region; and
at least one magnetic sensor positioned along an outer surface of the distal coupler in a groove;
a proximal coupler comprising:
a balloon securement region; and
a basket securement region proximal to the balloon securement region; and
at least one magnetic sensor secured to an outer surface of the proximal coupler;
a basket coupled to the basket securement regions of the distal and proximal couplers; and
a balloon positioned in the basket, the balloon coupled to the balloon securement regions of the distal and proximal couplers.

12. The medical device of claim 11, the basket further comprising one or more electrodes for ablation and/or mapping.

13. The medical device of claim 11, the basket comprising a plurality of splines, each spline comprising at least one overmolded section and at least one section without overmolding, wherein each of the at least one section without overmolding is an electrode.

14. The medical device of claim 11, wherein the balloon securement region of the distal coupler comprises overmolded portion formed of a material with a durometer hardness similar to a durometer hardness of the balloon for bonding the balloon to the distal coupler.

15. The medical device of claim 11, further comprising a first shaft coupled to the distal coupler, the shaft comprising:
a polymeric tube forming an inner layer of the shaft;
a braid positioned on, and coextensive with, the polymeric tube;
a coil positioned on a first end section of the braid.

16. A shaft for a medical device comprising:
a polymeric tube forming an inner layer of the shaft;
a braid positioned on, and coextensive with, the polymeric tube;
a coil positioned on a first end section of the braid; and
at least one polymer forming an outer layer of the shaft.

17. The shaft of claim 16, wherein the polymeric tube comprises polytetrafluoroethylene (PTFE), the at least one polymer comprises polyether block amide (PEBA).

18. The shaft of claim 17, wherein the at least one polymer comprises:
a first polymer with a first durometer hardness over the coil;
a second polymer with a second durometer hardness over an intermediate section of the braid; and
a third polymer with a third durometer hardness over a second end section of the braid.

19. The shaft of claim 18, wherein a length of the second end section is greater than a length of the first end section and less than a length of the intermediate section.

20. The shaft of claim 16, wherein the shaft is coupled to a distal coupler comprising a basket securement region.
